(19) United States
(12) Patent Application Publication
(10) Pub. No.: US 2002/0099070 A1
(43) Pub. Date: Jul. 25, 2002

(54) Dihydronaphthyridine potassium channel openers

(76) Inventor: Konstantinos Agrios, San Diego, CA (US)

Correspondence Address: Steven F. Weinstock, Abbott Laboratories, D-377/AP6D, 100 Abbott Park Road, Abbott Park, IL, 60064-6050, US

(21) Appl. No.: 09/921,037

(22) Filed: Aug. 2, 2001

Related U.S. Application Data
(60) Provisional application No. 60/222,505, filed on Aug. 2, 2000.

(51) Int. Cl. A61K 31/4745; C07D 471/02

(52) U.S. Cl. 514/300; 546/122

(57) ABSTRACT

Compounds of formula I

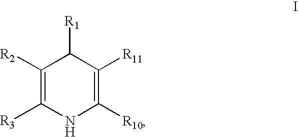

are useful in treating diseases prevented by or ameliorated with potassium channel openers. Also disclosed are potassium channel opening compositions and a method of opening potassium channels in a mammal.

This application claims priority to U.S. Provisional Application Serial No. 60/222,505, filed Aug. 2, 2000.

### TECHNICAL FIELD

Novel bicyclic dihydropyridine compounds and their derivatives can open potassium channels and are useful for treating a variety of medical conditions.

### BACKGROUND OF THE INVENTION

Potassium channels play an important role in regulating cell membrane excitability. When the potassium channels open, changes in the electrical potential across the cell membrane occur and result in a more polarized state. A number of diseases or conditions may be treated with therapeutic agents that open potassium channels; see (K. Lawson, Pharmacol. Ther., v. 70, pp. 39-63 (1996)); (D. R. Gehlert et al., Prog. Neuro-Psychopharmacol & Biol. Psychiat., v. 18, pp. 1093 -1102 (1994)); (M. Gopalakrishnan et al., Drug Development Research, v. 28, pp. 95-127 (1993)); (J. E. Freedman et al., The Neuroscientist, v. 2, pp. 145-152 (1996)); (D. E. Nurse et al., Br. J. Urol., v. 68 pp. 27-31 (1991)); (B. B. Howe et al., J. Pharmacol. Exp. Ther., v. 274 pp. 884-890 (1995)); (D. Spanswick et al., Nature, v. 390 pp. 521-25 (Dec. 4, 1997)); (Dompeling Vasa. Supplementum (1992) 3434); (WO9932495); (Grover, J Mol Cell Cardiol. (2000) 32, 677); and (Buchheit, Pulmonary Pharmacology & Therapeutics (1999) 12, 103). Such diseases or conditions include asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina and ischemia.

Bladder overactivity is a condition associated with the spontaneous, uncontrolled contractions of the bladder smooth muscle. Bladder overactivity thus is associated with or can cause diseases and conditions such as sensations of urgency, urinary incontinence, pollakiuria, bladder instability, nocturia, bladder hyerreflexia, and enuresis (Resnick, The Lancet (1995) 346, 94-99; Hampel, Urology (1997) 50 (Suppl 6A), 4-14; Bosch, BJU International (1999) 83 (Suppl 2), 7-9). Potassium channel openers (KCOs) act as smooth muscle relaxants. Because bladder overactivity and urinary incontinence can result from the spontaneous, uncontrolled contractions of the smooth muscle of the bladder, the ability of potassium channel openers to hyperpolarize bladder cells and relax bladder smooth muscle may provide a method to ameliorate or prevent bladder overactivity, pollakiuria, bladder instability, nocturia, bladder hyperreflexia, urinary incontinence, and enuresis (Andersson, Urology (1997) 50 (Suppl 6A), 74-84; Lawson, Pharmacol. Ther., (1996) 70, 39-63; Nurse., Br. J. Urol., (1991) 68, 27-31; Howe, J. Pharmacol. Exp. Ther., (1995) 274, 884-890; Gopalakrishnan, Drug Development Research, (1993) 28, 95-127).

The irritative symptoms of BPH (urgency, frequency, nocturia and urge incontinence) have been shown to be correlated to bladder instability (Pandita, The J. of Urology (1999) 162, 943). Therefore the ability of potassium channel openers to hyperpolarze bladder cells and relax bladder smooth muscle may provide a method to ameliorate or prevent the symptoms of BPH. (Andersson; Prostate (1997) 30: 202-215).

The excitability of corpus cavernosum smooth muscle cells is important in the male erectile process. The relaxation of corporal smooth muscle cells allows arterial blood to build up under pressure in the erectile tissue of the penis leading to erection (Andersson, Pharmacological Reviews (1993) 45,253). Potassium channels play a significant role in modulating human corporal smooth muscle tone, and thus, erectile capacity. By patch clamp technique, potassium channels have been characterized in human corporal smooth muscle cells (Lee, Int. J. Impot. Res. (1999) 11(4),179-188). Potassium channel openers are smooth muscle relaxants and have been shown to relax corpus cavemosal smooth muscle and induce erections (Andersson, Pharmacological Reviews (1993) 45, 253; Lawson, Pharmacol. Ther., (1996) 70, 39-63, Vick, J. Urol. (2000) 163: 202). Potassium channel openers therefore may have utility in the treatment of male sexual dysfunctions such as male erectile dysfunction, impotence and premature ejaculation.

The sexual response in women is classified into four stages: excitement, plateau, orgasm and resolution. Sexual arousal and excitement increase blood flow to the genital area, and lubrication of the vagina as a result of plasma transudation. Topical application of KCOs like minoxidil and nicorandil have been shown to increase clitoral blood flow (J. J. Kim, J. W. Yu, J. G. Lee, D. G. Moon, “Effects of topical K-ATP channel opener solution on clitoral blood flow”, J. Urol. (2000) 163 (4): 240). KCOs may be effective for the treatment of female sexual dysfunction including clitoral erectile insufficiency, vaginismus and vaginal engorgement (I. Goldstein and J. R. Berman., “Vasculogenic female sexual dysfunction: vaginal engorgement and clitoral erectile insufficiency syndromes”., Int. J. Impotence Res. (1998) 10:S84-S90), as KCOs can increase blood flow to female sexual organs.

Potassium channel openers may have utility as tocolytic agents to inhibit uterine contractions to delay or prevent premature parturition in individuals or to slow or arrest delivery for brief periods to undertake other therapeutic measures (Sanborn, Semin. Perinatol. (1995) 19, 31-40; Morrison, Am. J. Obstet. Gynecol. (1993) 169(5), 1277-85). Potassium channel openers also inhibit contractile responses of human uterus and intrauterine vasculature. This combined effect would suggest the potential use of KCOs for dysmenhorrea (Kostrzewska, Acta Obstet. Gynecol. Scand. (1996) 75(10), 886-91). Potassium channel openers relax uterine smooth muscle and intrauterine vasculature and therefore may have utility in the treatment of premature labor and dysmenorrhoea (Lawson, Pharmacol. Ther., (1996) 70, 39-63).

Potassium channel openers relax gastrointestinal smooth tissues and therefore may be useful in the treatment of functional bowel disorders such as irritable bowel syndrome (Lawson, Pharmacol. Ther., (1996) 70, 39-63).

Potassium channel openers relax airways smooth muscle and induce bronchodilation. Therefore potassium channel openers may be useful in the treatment of asthma and airways hyperreactivity (Lawson, Pharmacol. Ther., (1996) 70, 39-63; Buchheit, Pulmonary Pharmacology & Therapeutics (1999) 12, 103; Gopalakrishnan, Drug Development Research, (1993) 28, 95-127).

Neuronal hyperpolarization can produce analgesic effects. The opening of potassium channels by potassium channel openers and resultant hyperpolarization in the membrane of target neurons is a key mechanism in the effect of opioids. The peripheral antinociceptive effect of morphine results from activation of ATP-sensitive potassium channels, which causes hyperpolarization of peripheral terminals of primary afferents, leading to a decrease in action potential generation (Rodrigues, Br J Pharmacol (2000) 129(1), 110-4). Opening of K_{ATP} channels by potassium channel openers plays an important role in the antinociception mediated by alpha-2 adrenoceptors and mu opioid receptors. KCOs can potentiate the analgesic action of both morphine and dexmnedetomidine via an activation of K_{ATP} channels at the spinal cord level (Vergoni, Life Sci. (1992) 50(16), PL135-8; Asano, Anesth. Analg. (2000) 90(5), 1146-51). Thus, potassium channel openers can hyperpolarize neuronal cells and have shown analgesic effects. Potassium channel openers therefore may be useful as analgesics in the treatment of various pain states including but not limited to migraine and dyspareunia (Lawson, Pharmacol. Ther., (1996) 70, 39-63; Gopalakrishnan, Drug Development Research, (1993) 28, 95-127; Gehlert, Prog. Neuro-Psychopharmacol. & Biol. Psychiat., (1994) 18, 1093-1102).

Epilepsy results from the propagation of nonphysiologic electrical impulses. Potassium channel openers hyperpolarize neuronal cells and lead to a decrease in cellular excitability and have demonstrated antiepileptic effects. Therefore potassium channel openers may be useful in the treatment of epilepsy (Lawson, Pharmacol. Ther., (1996) 70, 39-63; Gopalakrishnan, Drug Development Research, (1993) 28, 95-127; Gehlert, Prog. Neuro-Psychopharmacol. & Biol. Psychiat., (1994) 18, 1093-1102).

Neuronal cell depolarization can lead to excitotoxicity and neuronal cell death. When this occurs as a result of acute ischemic conditions, it can lead to stroke. Long-term neurodegeneration can bring about conditions such as Alzheimer's and Parkinson's diseases. Potassium channel openers can hyperpolarize neuronal cells and lead to a decrease in cellular excitability. Activation of potassium channels has been shown to enhance neuronal survival. Therefore potassium channel openers may have utility as neuroprotectants in the treatment of neurodegenerative conditions and diseases such as cerebral ischemia, stroke, Alzheimer's disease and Parkinson's disease (Lawson, Phalmacol. Ther., (1996) 70, 39-63; Gopalakrishnan, Drug Development Research, (1993) 28, 95-127; Gehlert, Prog. Neuro-Psychopharmacol & Biol. Psychiat., (1994) 18, 1093-1102; Freedman, The Neuroscientist (1996)2, 145).

Potassium channel openers may have utility in the treatment of diseases or conditions associated with decreased skeletal muscle blood flow such as Raynaud's syndrome and intermittent claudication (Lawson, Pharmacol. Ther., (1996) 70, 39-63; Gopalakrishnan, Drug Development Research, (1993) 28, 95-127; Dompeling Vasa. Supplementum (1992) 3434; and WO9932495).

Potassium channel openers may be useful in the treatment of eating disorders such as obesity (Spanswick, Nature, (1997) 390, 521-25; Freedman, The Neuroscientist (1996) 2, 145).

Potassium channel openers have been shown to promote hair growth therefore potassium channel openers have utility in the treatment of hair loss and baldness also known as alopecia (Lawson, Pharmacol. Ther., (1996) 70, 39-63; Gopalakrishnan, Drug Development Research, (1993) 28, 95-127).

Potassium channel openers possess cardioprotective effects against myocardial injury during ischemia and reperfusion. (Garlid, Circ. Res. (1997) 81(6), 1072-82). Therefore, potassium channel openers may be useful in the treatment of heart diseases (Lawson, Pharmacol. Ther., (1996) 70, 39-63; Grover, J. Mol. Cell Cardiol. (2000) 32, 677).

Potassium channel openers, by hyperpolarization of smooth muscle membranes, can exert vasodilation of the collateral circulation of the coronary vasculature leading to increase blood flow to ischemic areas and could be useful for the coronary artery disease (Lawson, Pharmacol. Ther., (1996) 70, 39-63, Gopalakrishnan, Drug Development Research, (1993) 28, 95-127).

U.S. Pat. No. 4,567,268; U.S. Pat. No. 4,284,634; (Eur. J. Pharm. (1984) 105,229-237); (Poc. Natl. Acad. Sci. USA (1984) 81, 4824-4827); and (Tet. Lett. (1988) 29, 6835-6838); describe dihydrofuro[3,4-b]pyridin-5-ones. BE 893984 describes both dihydrofaro[3,4-b]pyridin-5-ones and tetrahydropyrano[4,3-b]pyridin-5-ones. DE 3605742 A1; U.S. Pat. No. 4,284,634; U.S. Pat. No. 5,025,011; EP 299727; and (Khim. Geterotsikl. Soedin. (1991) 1276); describe tetrahydropyrrolo[3,4-b]pyridin-5-ones. Ind. J. Chem. Sect. B (1995) 34B, 17-20 describes tetrahydro[1,6]naphthyridin-5-ones. Synthesis (1986) 859-860 describes 1,6-naphthyridines. U.S. Pat. No. 4,720,499; DE 3327650 A1; and DE 3502831 A1; describe dihydro[1,6]naphthyridinones.

The compounds of the present invention are novel and hyperpolarize cell membranes, open potassium channels and relax smooth muscle cells.

### SUMMARY OF THE INVENTION

In its principle embodiment, the present invention discloses compounds of formula I:

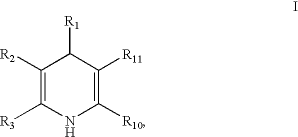

or a pharmaceutically acceptable salt thereof wherein,

R₁ is selected from the group consisting of aryl and heterocycle;

R₂ and R₃, together with the carbon atoms to which each is attached, are a ring selected from the group consisting of

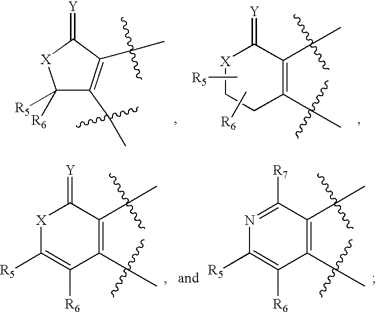

X is selected from the group consisting of O and NR₄;

Y is selected from the group consisting of O and S;

R₄ is selected from the group consisting of hydrogen, alkenyl, alkoxyalkyl, alkoxycarbonylalkyl, alkyl, alkylthioalkyl, alkynyl, carboxyalkyl, cyanoalkyl, hydroxyalkyl, mercaptoalkyl, and (NR₈R₉)alkyl wherein R₈ and R₉ are independently selected from the group consisting of hydrogen, alkyl, alkylcarbonyl, and formyl;

R₅ and R₆ are independently selected from the group consisting of hydrogen, alkenyl, alkoxy, alkyl, alkynyl, cyanoalkyl, haloalkyl, and halogen;

R₇ is selected from the group consisting of hydrogen, alkenyloxy, alkenylthio, alkoxy, alkylcarbonylalkoxy, alkylcarbonylalkylthio, alkylcarbonyloxy, alkylcarbonylthio, alkylthio, alkynyloxy, alkynylthio, cyanoalkoxy, cyanoalkylthio, halogen, and —NR₈R₉;

R₁₀ is selected from the group consisting of alkyl, aryl, arylalkyl, haloalkyl, heterocycle, heterocyclealkyl, hydroxyalkyl, and (NR₈R₉)alkyl; and

R₁₁ is selected from the group consisting of hydrogen, alkoxycarbonyl, alkyl, alkylcarbonyl, arylcarbonyl, carboxy, cyano, cyanoalkyl, haloalkyl, and haloalkylcarbonyl;

provided that when R₂ and R₃, together with the carbon atoms to which each is attached, are a ring selected from

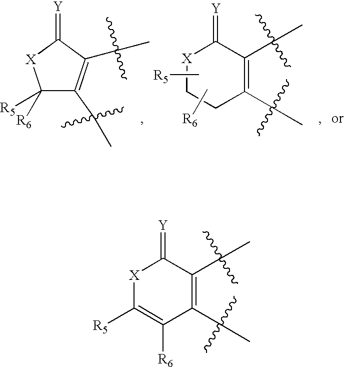

then R₁₁ is other than alkoxycarbonyl or carboxy; and

further provided that when R₂ and R₃, together with the carbon atoms to which each is attached, is

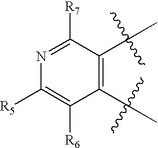

wherein R₅ and R₆ are hydrogen and R₇ is alkoxy, then R₁₁ is other than alkoxycarbonyl or carboxy.

### DETAILED DESCRIPTION OF THE INVENTION

All patents, patent applications, and literature references cited in the specification are herein incorporated by reference in their entirety. In the case of inconsistencies, the present disclosure, including definitions, will prevail.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof

In its principle embodiment, the present invention discloses compounds of formula I:

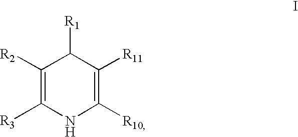

or a pharmaceutically acceptable salt thereof wherein,

R₁ is selected from the group consisting of aryl and heterocycle;

R₂ and R₃, together with the carbon atoms to which each is attached, are a ring selected from the group consisting of

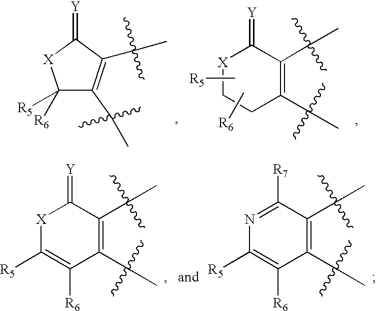

X is selected from the group consisting of O and NR₄;

Y is selected from the group consisting of O and S;

R₄ is selected from the group consisting of hydrogen, alkenyl, alkoxyalkyl, alkoxycarbonylalkyl, alkyl, alkylthioalkyl, alkynyl, carboxyalkyl, cyanoalkyl, hydroxyalkyl, mercaptoalkyl, and (NR₈R₉)alkyl wherein R₈ and R₉ are independently selected from the group consisting of hydrogen, alkyl, alkylcarbonyl, and formyl;

R₅ and R₆ are independently selected from the group consisting of hydrogen, alkenyl, alkoxy, alkyl, alkynyl, cyanoalkyl, haloalkyl, and halogen;

R₇ is selected from the group consisting of hydrogen, alkenyloxy, alkenylthio, alkoxy, alkylcarbonylalkoxy, alkylcarbonylalkylthio, alkylcarbonyloxy, alkylcarbonylthio, alkylthio, alkynyloxy, alkynylthio, cyanoalkoxy, cyanoalkylthio, halogen, and —NR₈R₉;

R₁₀ is selected from the group consisting of alkyl, aryl, arylalkyl, haloalkyl, heterocycle, heterocyclealkyl, hydroxyalkyl, and (NR₈R₉)alkyl; and

R₁₁ is selected from the group consisting of hydrogen, alkoxycarbonyl, alkyl, alkylcarbonyl, arylcarbonyl, carboxy, cyano, cyanoalkyl, haloalkyl, and haloalkylcarbonyl;

provided that when R₂ and R₃, together with the carbon atoms to which each is attached, are a ring selected from

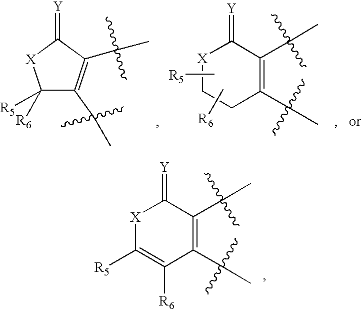

then R₁₁ is other than alkoxycarbonyl or carboxy; and

further provided that when R₂ and R₃, together with the carbon atoms to which each is attached, is

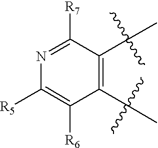

wherein R₅ and R₆ are hydrogen and R₇ is alkoxy, then R₁₁ is other than alkoxycarbonyl or carboxy.

In a preferred embodiment compounds of the present invention have formula I wherein R₅ is selected from hydrogen and alkyl; R₆ is selected from hydrogen and alkyl; R₇ is selected from alkoxy, alkylcarbonylalkylthio, alkylthio, and halogen; R₁₀ is selected from alkyl, aryl, and haloalkyl; R₁₁ is selected from alkylcarbonyl, arylcarbonyl, and cyano; and R₁, R₂, and R₃ are as defined in formula I.

In another preferred embodiment, compounds of the present invention have formula II

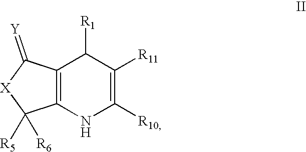

or a pharmaceutically acceptable salt thereof wherein X, Y, R₁, R₅, R₆, R₁₀ and R₁₁ are as defined in formula I.

In another preferred embodiment, compounds of the present invention have formula II wherein X is NR₄; Y is O; and R₅, R₆, R₁₀, R₁₁, R₁ and R₄ are as defined in formula I.

In another preferred embodiment, compounds of the present invention have formula II wherein X is NR₄; Y is O; R₅ is hydrogen; R₆ is hydrogen; R₁₀ is alkyl; R₁₁ is cyano; and R₁ and R₄ are as defined in formula I.

In another preferred embodiment, compounds of the present invention have formula II wherein X is O; Y is O; and R₅, R₆, R₁₀, R₁₁ and R₁ are as defined in formula I.

In another preferred embodiment, compounds of the present invention have formula II wherein X is O; Y is O; R₅ is hydrogen; R₆ is hydrogen; R₁₀ is alkyl; R₁₁ is cyano; and R₁ is as defined in formula I.

In another preferred embodiment, compounds of the present invention have formula II wherein X is NR₄; Y is S; and R₅, R₆, R₁₀, R₁₁, R₁ and R₄ are as defined in formula I.

In another preferred embodiment, compounds of the present invention have formula II wherein X is NR₄; Y is S; R₅ is hydrogen; R₆ is hydrogen; R₁₀ is alkyl; R₁₁ is cyano; and R₁ and R₄ are as defined in formula I.

In another preferred embodiment, compounds of the present invention have formula II wherein X is O; Y is S; and R₅, R₆, R₁₀, R₁₁ and R₁ are as defined in formula I.

In another preferred embodiment, compounds of the present invention have formula II wherein X is O; Y is S; R₅ is hydrogen; R₆ is hydrogen; R₁₀ is alkyl; R₁₁ is cyano; and R₁ is as defined in formula I.

In another preferred embodiment, compounds of the present invention have formula III

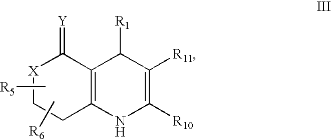

or a pharmaceutically acceptable salt thereof wherein X, Y, R₁, R₅, R₆, R₁₀ and R₁₁ are as defined in formula I.

In another preferred embodiment, compounds of the present invention have formula III wherein X is NR₄; Y is O; and R₅, R₆, R₁₀, R₁₁, R₁ and R₄ are as defined in formula I.

In another preferred embodiment, compounds of the present invention have formula III wherein X is NR₄; Y is O; R₅ is hydrogen; R₆ is hydrogen; R₁₀ is alkyl; R₁₁ is cyano; and R₁ and R₄ are as defined in formula I.

In another preferred embodiment, compounds of the present invention have formula III wherein X is NR₄; Y is O; R₅ is hydrogen; R₆ is hydrogen; R₁₀ is alkyl; R₁₁ is alkylcarbonyl; and R₁ and R₄ are as defined in formula I.

In another preferred embodiment, compounds of the present invention have formula III wherein X is NR₄; Y is O; R₅ is hydrogen; R₆ is hydrogen; R₁₀ is aryl; R₁₁ is arylcarbonyl; and R₁ and R₄ are as defined in formula I.

In another preferred embodiment, compounds of the present invention have formula III wherein X is NR₄; Y is O; R₅ is hydrogen; R₆ is hydrogen; R₁₀ is haloalkyl; R₁₁ is alkylcarbonyl; and R₁ and R₄ are as defined in formula I.

In another preferred embodiment, compounds of the present invention have formula III wherein X is NR₄; Y is S; and R₅, R₁₀, R ,R, R₁ and R₄ are as defined in formula I.

In another preferred embodiment, compounds of the present invention have formula III wherein X is O; Y is O; and R₅, R₆, R₁₀, R₁₁ and R₁ are as defined in formula I.

In another preferred embodiment, compounds of the present invention have formula III wherein X is O; Y is O; R₅ is hydrogen; R₆ is hydrogen; R₁₀ is alkyl; R₁₁ is cyano; and R₁ is as defined in formula I.

In another preferred embodiment, compounds of the present invention have formula III wherein X is O; Y is S; and R₅, R₆, R₁₀, R₁₁ and R₁ are as defined in formula I.

In another preferred embodiment, compounds of the present invention have formula IV

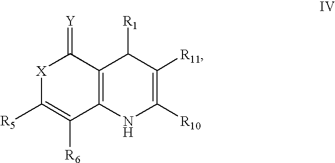

or a pharmaceutically acceptable salt thereof wherein X, Y, R₁, R₅, R₆, R₁₀ and R₁₁ are as defined in formula I.

In another preferred embodiment, compounds of the present invention have formula IV wherein X is NR₄; Y is O; and R₅, R₆, R₁₀, R₁₁, R₁ and R₄ are as defined in formula I.

In another preferred embodiment, compounds of the present invention have formula IV wherein X is NR₄; Y is O; R₅ is hydrogen; R₆ is hydrogen; R₁₀ is alkyl; R₁₁ is cyano; and R₁ and R₄ are as defined in formula I.

In another preferred embodiment, compounds of the present invention have formula IV wherein X is NR₄; Y is O; R₅ is hydrogen; R₆ is hydrogen; R₁₀ is alkyl; R₁₁ is alkylcarbonyl; and R₁ and R₄ are as defined in formula I.

In another preferred embodiment, compounds of the present invention have formula IV wherein X is NR₄; Y is O; R₅ is hydrogen; R₆ is hydrogen; R₁₀ is haloalkyl; R₁ is alkylcarbonyl; and R₁ and R₄ are as defined in formula I.

In another preferred embodiment, compounds of the present invention have formula IV wherein X is NR₄; Y is O; R₅ is hydrogen; R₆ is hydrogen; R₁₀ is aryl; R₁₁ is arylcarbonyl; and R₁ and R₄ are as defined in formula I.

In another preferred embodiment, compounds of the present invention have formula IV wherein X is NR₄ Y is S; and R₅, R₆, R₁₀, R₁₁, R₁ and R₄ are as defined in formula I.

In another preferred embodiment, compounds of the present invention have formula IV wherein X is NR₄; Y is S; R₅ is hydrogen; R₆ is hydrogen; R₁₀ is alkyl; R₁₁ is cyano; and R₁ and R₄ are as defined in formula I.

In another preferred embodiment, compounds of the present invention have formula IV wherein X is O; Y is O; and R₅, R₆, R₁₀, R₁₁ and R₁ are as defined in formula I.

In another preferred embodiment, compounds of the present invention have formula IV wherein X is O; Y is O; R₅is hydrogen; R₆ is hydrogen; R₁₀ is alkyl; R₁₁ is cyano; and R₁ is as defined in formula I.

In another preferred embodiment, compounds of the present invention have formula IV wherein X is O; Y is S; and R₅, R₆, R₁₀, R₁₁ and R₁ are as defined in formula I.

In another preferred embodiment, compounds of the present invention have formula V

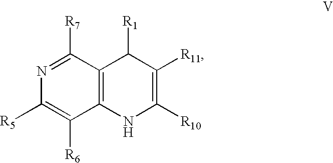

or a pharmaceutically acceptable salt thereof wherein R₁, R₅, R₆, R₇, R₁₀ and R₁₁ are as defined in formula I.

In another preferred embodiment, compounds of the present invention have formula V wherein R₅is hydrogen; R₆ is hydrogen; R₁₀ is alkyl; R₁₁ is cyano; and R₁ and R₇ are as defined in formula I.

In another preferred embodiment, compounds of the present invention have formula V wherein R₅ is hydrogen; R₆ is hydrogen; R₇ is hydrogen, alkenyloxy, alkenylthio, alkylcarbonylalkoxy, alkylcarbonylalkylthio, alkylcarbonylthio, alkylthio, alkynyloxy, alkynylthio, cyanoalkoxy, cyanoalkylthio, halogen, and —NR₈R₉; R₁₀ is alkyl; R₁₁ is alkoxycarbonyl; and R₈, R₉ and R₁ are as defined in formula I.

In another preferred embodiment, compounds of the present invention have formula V wherein R₅ is hydrogen; R₆ is hydrogen; R₇ is halogen; R₁₀ is alkyl; R₁₁ is alkoxycarbonyl; and R₁ is as defined in formula I.

Another embodiment of the present invention relates to pharmaceutical compositions comprising a therapeutically effective amount of a compound of formula I-V or a pharmaceutically acceptable salt, ester, amide, or prodrug thereof in combination with a pharmaceutically acceptable carrier.

Another embodiment of the invention relates to a method of treating male sexual dysfunction including, but not limited to, male erectile dysfunction and premature ejaculation, comprising administering a therapeutically effective amount of a compound of formula I-V or a pharmaceutically acceptable salt, ester, amide, or prodrug thereof.

Another embodiment of the invention relates to a method of treating female sexual dysfunction including, but not limited to, female anorgasmia, clitoral erectile insufficiency, vaginal engorgement, dyspareunia, and vaginismus comprising administering a therapeutically effective amount of a compound of formula I-V or a pharmaceutically acceptable salt, ester, amide, or prodrug thereof

Yet another embodiment of the invention relates to a method of treating asthma, epilepsy, Raynaud's syndrome, intermittent claudication, migraine, pain, bladder overactivity, pollakiuria, bladder instability, nocturia, bladder hyperreflexia, eating disorders, urinary incontinence, enuresis, functional bowel disorders, neurodegeneration, benign prostatic hyperplasia (BPH), dysmenorrhea, premature labor, alopecia, cardioprotection, and ischemia comprising administering a therapeutically effective amount of a compound of formula I-V or a pharmaceutically acceptable salt, ester, amide, or prodrug thereof

### Definition of Terms

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenyloxy,” as used herein, refers to an alkenyl group, as defined herein, appended to the parent molecular moiety through an oxy moiety, as defined herein. Representative examples of alkenyloxy include, but are not limited to, allyloxy, 2-butenyloxy and 3-butenyloxy.

The term “alkenylthio,” as used herein, refers to an alkenyl group, as defined herein, appended to the parent molecular moiety through a thio moiety, as defined herein. Representative examples of alkenylthio include, but are not limited to, allylsulfanyl, 2-butenylsulfanyl and 3-butenylsulfanyl.

The term “alkoxy,” as used herein, refers to an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, pentyloxy, and hexyloxy.

The term “alkoxyalkoxy,” as used herein, refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl,” as used herein, refers to an alkoxyalkoxy group, as defined herein, appended to the parent molecular moiety through an alkyl group, as defined herein. Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl, ” as used herein, refers to an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkoxy,” as used herein, refers to an alkylcarbonyl group, as defined herein, appended to the parent molecular moiety through an alkoxy group, as defined herein. Representative examples of alkylcarbonylalkoxy include, but are not limited to, 2-oxopropoxy and 3-oxobutoxy.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonylalkylthio,” as used herein, refers to an alkylcarbonyl group, as defined herein, appended to the parent molecular moiety through an alkylthio group, as defined herein. Representative examples of alkylcarbonylalkylthio include, but are not limited, (2-oxopropyl)sulfanyl, (2-oxobutyl)sulfanyl, and (3-oxobutyl)sulfanyl.

The term “alkylcarbonyloxy,” as used herein, refers to an alkylcarbonyl group, as defined herein, appended to the parent molecular moiety through an oxy moiety, as defined herein. Representative examples of alkylcarbonyloxy include, but are not limited to, acetyloxy, ethylcarbonyloxy, and tert-butylcarbonyloxy.

The term “alkylcarbonylthio,” as used herein, refers to an alkylcarbonyl group, as defined herein, appended to the parent molecular moiety through a thio moiety, as defined herein. Representative examples of alkylcarbonylthio include, but are not limited to, acetylsulfanyl and propionylsulfanyl.

The term “alkylsulfinyl,” as used herein, refers to an alkyl group, as defined herein, appended to the parent molecular moiety through a sulfinyl group, as defined herein. Representative examples of alkylsulfinyl include, but are not limited, methylsulfinyl, and ethylsulfinyl.

The term “alkylsulfonyl,” as used herein, refers to an alkyl group, as defined herein, appended to the parent molecular moiety through a sulfonyl group, as defined herein. Representative examples of alkylsulfonyl include, but are not limited, methylsulfonyl, and ethylsulfonyl.

The term “alkylthio,” as used herein, refers to an alkyl group, as defined herein, appended to the parent molecular moiety through a thio moiety, as defined herein. Representative examples of alkylthio include, but are not limited, methylsulfanyl, ethylsulfanyl, tert-butylsulfanyl, and hexylsulfanyl.

The term “alkylthioalkyl,” as used herein, refers to an alkylthio group, as defined herein, appended to the parent molecular moiety through an alkyl moiety, as defined herein. Representative examples of alkylthioalkyl include, but are not limited, (methylsulfanyl)methyl, (ethylsulfanyl)methyl, 2-(tert-butylsulfanyl)ethyl, and (hexylsulfanyl)methyl.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkynyloxy,” as used herein, refers to an alkynyl group, as defined herein, appended to the parent molecular moiety through an oxy moiety, as defined herein. Representative examples of alkynyloxy include, but are not limited, 2-propynyloxy and 2-butynyloxy.

The term “alkynylthio,” as used herein, refers to an alkynyl group, as defined herein, appended to the parent molecular moiety through a thio moiety, as defined herein. Representative examples of alkynylthio include, but are not limited, 2-propynylsulfanyl and 2-butynylsulfanyl.

The term “aryl,” as used herein, refers to an aromatic monocyclic carbocyclic ring system or a bicyclic carbocyclic fused ring system having one or more aromatic rings. Representative examples of aryl include, azulenyl, indanyl, indenyl, naphthyl, phenyl, and tetrahydronaphthyl.

The aryl groups of this invention can be substituted with 1, 2, 3, 4, or 5 substituents independently selected from alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, aryl, azido, arylalkoxy, arylalkyl, aryloxy, carboxy, cyano, formyl, 2-furyl, 3-faryl, halogen, haloalkyl, haloalkoxy, hydroxy, hydroxyalkyl, mercapto, nitro, sulfo, sulfonate, —NR_{A}R_{B}, and —C(O)NR_{A}R_{B} wherein R_{A} and R_{B} are independently selected from hydrogen, alkyl, alkylcarbonyl, arylalkyl and formyl.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl,” as used herein, refers to an arylalkoxy group, as defined herein, appended to the parent molecular moiety through a carbonyl group, as defined herein. Representative examples of arylalkoxycarbonyl include, but are not limited to, benzyloxycarbonyl, and naphth-2-ylmethoxycarbonyl.

The term “arylalkyl,” as used herein, refers to an aryl group, as defined herein, appended to the parent molecular moiety through an alkyl group, as defined herein.

Representative examples of arylalkyl include, but are not limited to, benzyl, 2-phenylethyl, 3-phenylpropyl, and 2-naphth-2-ylethyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl and naphthoyl.

The term “aryloxy,” as used herein, refers to an aryl group, as defined herein, appended to the parent molecular moiety through an oxy moiety, as defined herein. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyalkyl,” as used herein, refers to an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl, and 3-bromophenoxymethyl.

The term “azido,” as used herein, refers to a —N₃ group.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkoxy,” as used herein, refers to a cyano group, as defined herein, appended to the parent molecular moiety through an alkoxy group, as defined herein. Representative examples of cyanoalkoxy include, but are not limited to, 2-cyanoethoxy and cyanomethoxy.

The term “cyanoalkylthio,” as used herein, refers to a cyanoalkyl group, as defined herein, appended to the parent molecular moiety through a thio group, as defined herein. Representative examples of cyanoalkylthio include, but are not limited to, (cyanomethyl)sulfanyl and (2-cyanoethyl)sulfanyl.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl,” as used herein, refers to a saturated cyclic hydrocarbon group containing from 3 to 8 carbons. Representative examples of cycloalkyl include, but are not limited to, cyclopropyl, cyclobutyl, cyclopentyl, cyclohexyl, cycloheptyl, and cyclooctyl.

The term “cycloalkylalkyl,” as used herein, refers to cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein.

Representative examples of haloalkoxy include, but are not limited to, chloromethoxy, 2,2,2-trifluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkylcarbonyl,” as used herein, refers to a haloalkyl group, as defined herein, appended to the parent molecular moiety through a carbonyl group, as defined herein. Representative examples of haloalkylcarbonyl include, but are not limited to, trifluoroacetyl and chloroacetyl.

The term “heterocycle,” as used herein, refers to a monocyclic- or a bicyclic-ring system. Monocyclic ring systems are exemplified by any 5- or 6-membered ring containing 1, 2, 3, or 4 heteroatoms independently selected from oxygen, nitrogen and sulfur. The 5-membered ring has from 0-2 double bonds and the 6-membered ring has from 0-3 double bonds. Representative examples of monocyclic ring systems include, but are not limited to, azetidine, azepine, aziridine, diazepine, 1,3-dioxolane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, and trithiane. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, and thiopyranopyridine.

The heterocycle groups of this invention can be substituted with 1, 2,or 3 substituents independently selected from alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, aryl, azido, arylalkoxy, arylalkoxycarbonyl, arylalkyl, aryloxy, carboxy, cyano, formyl, halogen, haloalkyl, haloalkoxy, hydroxy, hydroxyalkyl, mercapto, nitro, sulfo, sulfonate, —NR_{A}R_{B}, and —C(O)NR_{A}R_{B} wherein R_{A} and R_{B} are independently selected from hydrogen, alkyl, alkylcarbonyl, arylalkyl and formyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyrid-3-ylmethyl, and 2-pyrimidin-2-ylpropyl.

The term “hydroxy,” as used herein, refers to an —OH group.

The term “hydroxyalkyl,” as used herein, refers to a hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, and 2-ethyl-4-hydroxyheptyl.

The term “lower alkyl,” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, and tert-butyl.

The term “mercapto,” as used herein, refers to a —SH group.

The term “mercaptoalkyl,” as used herein, refers to a mercapto group, as defined herein, appended to the parent molecular moiety through an alkyl group, as defined herein. Representative examples of mercaptoalkyl include, but are not limited to, sulfanylmethyl, 2-sulfanylethyl, and 3-sulfanylpropyl.

The term “nitro,” as used herein, refers to a —NO₂ group.

The term “N-protecting group” or “nitrogen protecting group,” as used herein, refers to those groups intended to protect an amino group against undesirable reactions during synthetic procedures. N-protecting groups comprise carbamates, amides including those containing hetero arylgroups, N-alkyl derivatives, amino acetal derivatives, N-benzyl derivatives, imine derivatives, enamine derivatives and N-heteroatom derivatives. Preferred N-protecting groups are formyl, acetyl, benzoyl, pivaloyl, phenylsulfonyl, benzyl, triphenylmethyl (trityl), t-butyloxycarbonyl (Boc), benzyloxycarbonyl (Cbz). Commonly used N-protecting groups are disclosed in T. H. Greene and P. G. M. Wuts, Protective Groups in Organic Synthesis, 2nd edition, John Wiley & Sons, New York (1991), which is hereby incorporated by reference.

The term “—NR₈R₉,” as used herein, refers to two groups, R₈ and R₉, which are appended to the parent molecular moiety through a nitrogen atom. R₈ and R₉ are independently selected from hydrogen, alkyl, alkylcarbonyl and formyl. Representative examples of —NR₈R₉ include, but are not limited to, amino, methylamino, acetylamino, and acetylmethylamino.

The term “(R₈R₉)alkyl,” as used herein, refers to a —NR₈R₉ group as defined herein, appended to the parent molecular moiety through an alkyl group as defined herein. Representative examples of (NR₈R₉)alkyl include, but are not limited to, aminomethyl, (methylamino)methyl, 2-(acetylamino)ethyl, and 2-(acetylmethylamino)ethyl.

The term “oxo,” as used herein, refers to a ═O moiety.

The term “oxy,” as used herein, refers to a —O— moiety.

The term “sulfinyl,” as used herein, refers to a —S(O)— group.

The term “sulfo,” as used herein, refers to a —SO₃H group.

The term “sulfonate,” as used herein, refers to —S(O)₂OR₉₆ group, wherein R₉₆ is selected from alkyl, aryl, and arylalkyl, as defined herein.

The term “sulfonyl,” as used herein, refers to a —SO₂— group.

The term “tautomer” as used herein refers to a proton shift from one atom of a molecule to another atom of the same molecule wherein two or more structurally distinct compounds are in equilibrium with each other.

The term “thio,” as used herein, refers to a —S— moiety.

Preferred compounds of formula I include, but are not limited to:

4-(3-bromo-4-fluorophenyl)-3-cyano-2-methyl-4,6,7,8-tetrahydro[1,6]naphthyridin-5(1H)-one;

(+) 4-(3-bromo-4-fluorophenyl)-3-cyano-2-methyl-4,6,7,8-tetrahydro[1,6]naphthyridin-5(1H)-one;

(−) 4-(3-bromo-4-fluorophenyl)-3-cyano-2-methyl-4,6,7,8-tetrahydro[1,6]naphthyridin-5(1H)-one;

4-(3-bromo-4-fluorophenyl)-3-cyano-2-methyl-4,6-dihydro[1,6]naphthyridin-5(1H)-one;

(+) 4-(3-bromo-4-fluorophenyl)-3-cyano-2-methyl-4,6-dihydro[1,6]naphthyridin-5(1H)-one;

(−) 4-(3-bromo-4-fluorophenyl)-3-cyano-2-methyl-4,6-dihydro[1,6]naphthyridin-5(1H)-one;

4-(3-bromo-4-fluorophenyl)-3-cyano-2,6-dimethyl-4,6-dihydro[1,6]naphthyridin-5(1H)-one;

4-(3,4-dichlorophenyl)-3-cyano-2-methyl4,6,7,8-tetrahydro[1,6]naphthyridin-5(1H)-one;

4-(3-nitrophenyl)-3-cyano-2-methyl-4,6,7,8-tetrahydro[1,6]naphthyridin-5(1H)-one;

4-(4-chloro-3-nitrophenyl)-3-cyano-2-methyl-4,6,7,8-tetrahydro[1,6]naphthyridin-5(1H)-one;

4-(3,4-dibromophenyl)-3-cyano-2-methyl-4,6,7,8-tetrahydro[1,6]naphthyridin-5(1H)-one;

4-(3,4-difluorophenyl)-3-cyano-2-methyl-4,6,7,8-tetrahydro[1,6]naphthyridin-5(1H)-one;

4-[4-fluoro-3-(trifluoromethyl)phenyl]-3-cyano-2-methyl-4,6,7,8-tetrahydro[1,6]naphthyridin-5(1H)-one;

4-(2,4,5-trifluorophenyl)-3-cyano-2-methyl-4,6,7,8-tetrahydro[1,6]naphthyridin-5(1H)-one;

4-(3-chloro-4-fluorophenyl)-3-cyano-2-methyl-4,6,7,8-tetrahydro[1,6]naphthyridin-5(1H)-one;

4-[4-chloro-3-(trifluoromethyl)phenyl]-3-cyano-2-methyl-4,6,7,8-tetrahydro[1,6]naphthyridin-5(1H)-one;

3-acetyl-4-(3-bromo-4-fluorophenyl)-2-methyl-4,6,7,8-tetrahydro[1,6]naphthyridin-5(1H)-one;

3-acetyl-4-(3-bromo-4-fluorophenyl)-2-methyl-4,6-dihydro[1,6]naphthyridin-5(1H)-one;

(+) 3-acetyl-4-(3-bromo-4-fluorophenyl)-2-methyl-4,6-dihydro[1,6]naphthyridin-5(1H)-one;

(−) 3-acetyl-4-(3-bromo-4-fluorophenyl)-2-methyl-4,6-dihydro[1,6]naphthyridin-5(1H)-one;

3-benzoyl-4-(3-bromo-4-fluorophenyl)-2-phenyl-4,6,7,8-tetrahydro[1,6]naphthyridin-5(1H)-one;

3-benzoyl-4-(3-bromo-4-fluorophenyl)-2-phenyl-4,6-dihydro[1,6]naphthyridin-5(1H)-one;

4-(3-bromo-4-fluorophenyl)-2-methyl-3-(3-methylbutanoyl)-4,6,7,8-tetrahydro[1,6]naphthyridin-5(1H)-one;

4-(3-bromo-4-fluorophenyl)-2-methyl-3-(3-methylbutanoyl)-4,6-dihydro[1,6]naphthyridin-5(1H)-one;

4-(3-bromo-4-fluorophenyl)-3-(2,2-dimethylpropanoyl)-2-(trifluoromethyl)-4,6,7,8-tetrahydro[1,6]naphthyridin-5(1H)-one;

4-[4-fluoro-3-(trifluoromethyl)phenyl]-3-cyano-2-methyl-1,4,6,7-tetrahydro-5H-pyrrolo[3,4-b]pyridin-5-one;

4-[4-fluoro-3-(trifluoromethyl)phenyl]-3-cyano-2-methyl-1,4,7,8-tetrahydro-5H-pyrano[4,3-b]pyridin-5-one;

(+) 4-[4-fluoro-3-(trifluoromethyl)phenyl]-3-cyano-2-methyl-1,4,7,8-tetrahydro-5H-pyrano[4,3-b]pyridin-5-one;

(−) 4-[4-fluoro-3-(trifluoromethyl)phenyl]-3-cyano-2-methyl-1,4,7,8-tetrahydro-5H-pyrano[4,3-b]pyridin-5-one;

4-(3-bromo-4-fluorophenyl)-3-cyano-2-methyl-1,4,7,8-tetrahydro-5H-pyrano[4,3-b]pyridin-5-one;

4-(3-bromo-4-fluorophenyl)-3-methoxycarbonyl-2-methyl-1,4,7,8-tetrahydro-5H-pyrano[4,3-b]pyridin-5-one;

4-[4-fluoro-3-(trifluoromethyl)phenyl]-3-methoxycarbonyl-2-methyl-1,4,7,8-tetrahydro-5H-pyrano[4,3-b]pyridin-5-one;

4-(3-bromo-4-fluorophenyl)-5-chloro-3-(methoxycarbonyl)-2-methyl-1,4-dihydro[1,6]naphthyridine;

4-(3-bromo-4-fluorophenyl)-5-chloro-3-cyano-2-methyl-1,4-dihydro[1,6]naphthyridine;

(+) 4-(3-bromo-4-fluorophenyl)-5-chloro-3-cyano-2-methyl-1,4-dihydro[1,6]naphthyridine;

(−) 4-(3-bromo-4-fluorophenyl)-5-chloro-3-cyano-2-methyl-1,4-dihydro[1,6]naphthyridine;

4-(3-bromo-4-fluorophenyl)-3-cyano-2-methyl-4,6-dihydro[1,6]naphthyridine-5(1H)-thione;

4-(3-bromo-4-fluorophenyl)-3-cyano-2-methyl-5-[(2-oxobutyl)sulfanyl]-1,4-dihydro[1,6]naphthyridine;

4-(3-bromo-4-fluorophenyl)-3-cyano-2-methyl-5-(methylsulfanyl)-1,4-dihydro[1,6]naphthyridine;

4-(3-bromo-4-fluorophenyl)-3-cyano-2-methyl-5-methoxy-1,4-dihydro[1,6]naphthyridine;

4-(3-bromo-4-fluorophenyl)-5-chloro-3-(ethoxycarbonyl)-2-methyl-1,4-dihydro[1,6]naphthyridine;

(+) 4-(3-bromo-4-fluorophenyl)-5-chloro-3-(ethoxycarbonyl)-2-methyl-1,4-dihydro[1,6]naphthyridine;

(−) 4-(3-bromo-4-fluorophenyl)-5-chloro-3-(ethoxycarbonyl)-2-methyl-1,4-dihydro[1,6]naphthyridine;

4-(3-bromo-4-fluorophenyl)-3-cyano-6-(cyanomethyl)-2-methyl-4,6-dihydro[1,6]naphthyridin-5(1H)-one;

4-(3-bromo-4-fluorophenyl)-6-(cyanomethyl)-3-(methoxycarbonyl)-2-methyl-4,6-dihydro[1,6]naphthyridin-5(1H)-one;

4-(3-bromo-4-fluorophenyl)-3-(2,2-dimethylpropanoyl)-2-(trifluoromethyl)-4,6-dihydro[1,6]naphthyridin-5(1H)-one and pharmaceutically acceptable salts, esters, amides, or prodrugs thereof

### Abbreviations

Abbreviations which have been used in the descriptions of the schemes and the examples that follow are: AcOH for acetic acid, BF₃OEt₂ for boron trifluoride diethyl ether complex, Boc for tert-butoxycarbonyl, (Boc)₂O for di-tert-butyl dicarbonate, Bn for benzyl, Bu for butyl, n-BuLi for n-butyllithium, DMAP for 4-dimethylaminopyridine, DMF for N,N-dimethylformamide, DMSO for dimethylsulfoxide, Et for ethyl, EtOAc for ethyl acetate, EtOH for ethanol, LAH for lithium aluminum hydride, LDA for lithium diisopropylamide, Me for methyl, MeCN for acetonitrile, MeOH for methanol, NBS for N-bromosuccinimide, PPTS for pyridinium p-toluenesulfonate, pyr for pyridine, rt for room temperature or ambient temperature, TFA for trifluoroacetic acid, THF for tetrahydrofuran, p-TsOH for para-toluenesulfonic acid monohydrate.

### Preparation of Compounds of The Invention

The compounds and processes of the present invention will be better understood in connection with the following synthetic schemes and methods which illustrate a means by which the compounds of the invention can be prepared.

The compounds of this invention can be prepared by a variety of synthetic routes. Representative procedures are shown in Schemes 1-24.

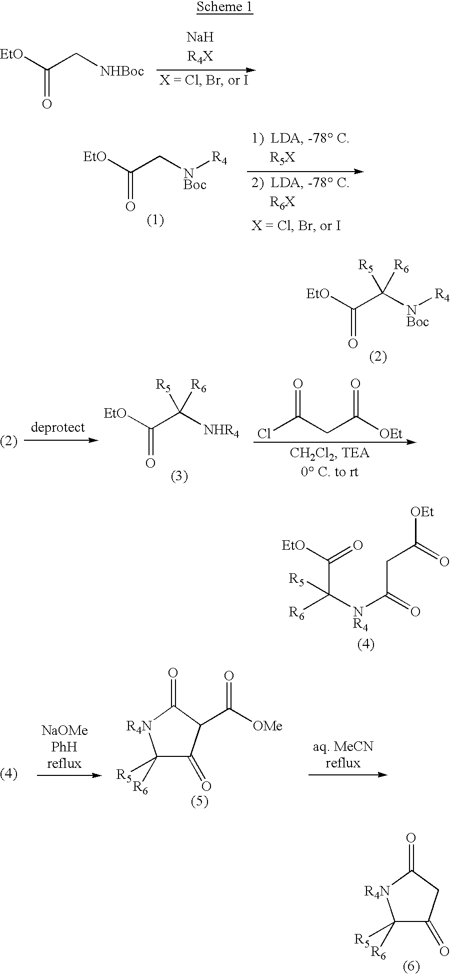

2,4-Pyrrolidinediones of general formula (6), wherein R₅ and R₆ are as defined in formula I, may be prepared as described in Scheme 1 or as described in (Lowe and Yeung, J. Chem. Soc., Perkin Trans. (I) (1973) 2907-2910). Ethyl [(tert-butoxycarbonyl)amino]acetate may be treated with sodium hydride and alkylating agents such as iodomethane, allyl bromide, propargyl bromide or bromoacetonitrile to provide esters of general formula (1). Esters of general formula (1) may also be obtained commercially such as ethyl [(tert-butoxycarbonyl)(methyl)amino]acetate. Esters of general formula (1) may be treated with lithium diisopropylamide at −78° C. to 0° C. and alkylating agents such as iodomethane, allyl bromide, propargyl bromide or bromoacetonitrile in a solvent such as THF to provide mono or disubstituted esters of general formula (2). Chiral mono substituted esters of general formula (2), wherein at least one of R₅ and R₆ is hydrogen, may also be obtained commercially such as (L) or (D) α-amino acids (alanine, valine, leucine, and isoleucine). Chiral α-amino acids may also be prepared as described in (Myers et al., JACS (1997) 119, 656-673). Mono or disubstituted esters of general formula (2) maybe deprotected using TFA/CH₂Cl₂ (1:1) or 4N HCl in 1,4-dioxane to provide amines of general formula (3). Amines of general formula (3) may be treated with ethyl 3-chloro-3-oxopropionate to provide diesters of general formula (4). Diesters of general formula (4) may be treated with sodium methoxide in benzene at reflux to provide lactams of general formula (5). Lactams of general formula (5) may be heated at reflux in aqueous acetonitrile to provide 2,4-pyrrolidinediones of general formula (6).

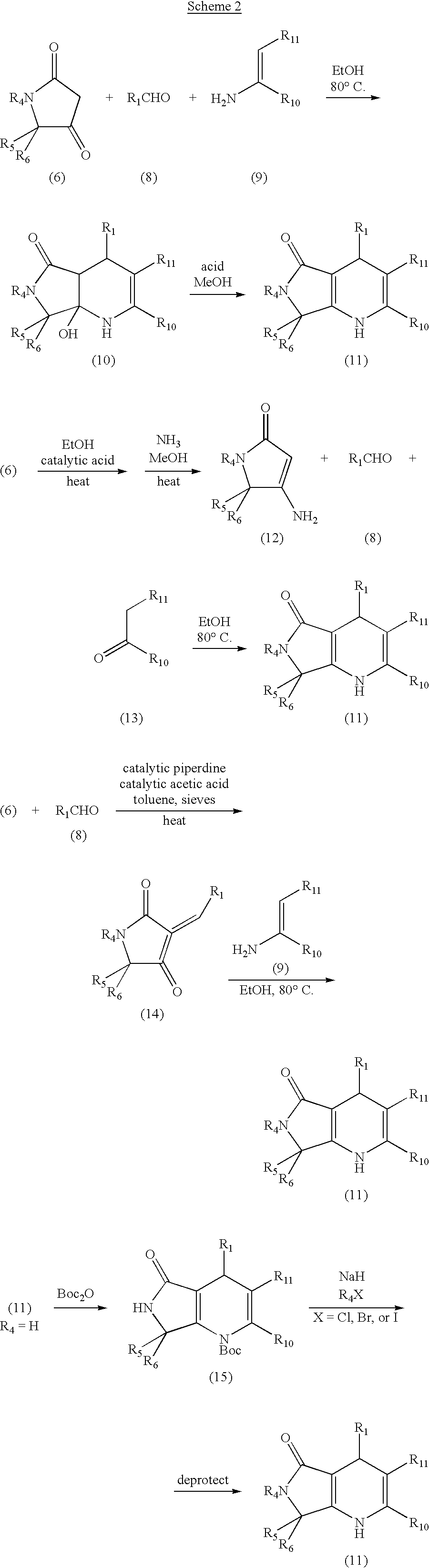

Pyrrolo[3,4-b]pyridinones of general formula (11), wherein R₁, R₄, R₅, R₆, R₁₀, and R₁₁ are as defined in formula I, may be prepared as described in Scheme 2. 2,4-Pyrrolidinediones of general formula (6), from Scheme 1, may be treated with aldehydes of general formula (8) and enamines of general formula (9) in a solvent such as ethanol at 80° C. to provide hydroxy-pyrrolo[3,4-b]pyridinones of general formula (10). Hydroxy-pyrrolo[3,4-b]pyridinones of general formula (10) may be treated with acid and methanol to provide pyrrolo[3,4-b]pyridinones of general formula (11).

Alternatively, 2,4-pyrrolidinediones of general formula (6) may be treated with an alcohol such as ethanol, a catalytic amount of acid, and heat to provide the vinyl ether which may then be treated with ammonia to provide enaminones of general formula (12). Enaminones of general formula (12) may be treated with aldehydes of general formula (8) and ketones of general formula (13) in ethanol at 80° C. to provide pyrrolo[3,4-b]pyridinones of general formula (11).

Alternatively, 2,4-pyrrolidinediones of general formula (6) may be treated with a catalytic amount of piperidine, pyrrolidine, or morpholine, a catalytic amount of acid such as acetic acid, and 4 Å molecular sieves in toluene with heating to provide α,β-unsaturated diones of general formula (14). α,β-Unsaturated diones of general formula (14) may be treated with enamines of general formula (9) to provide pyrrolo[3,4-b]pyridinones of general formula (11). In the case where a hemiaminal is isolated, an additional step at ambient temperature or at an elevated temperature in the presence or the absence of an acid such as hydrochloric acid or para-toluenesulfonic acid may be necessary to provide pyrrolo[3,4-b]pyridinones of general formula (11).

Pyrrolo[3,4-b]pyridinones of general formula (11), wherein R₄ is hydrogen, may be treated with a nitrogen protecting group reagent such as di-tert-butyl dicarbonate and DMAP in 1,4-dioxane to provide pyrrolo[3,4-b]pyridinones of general formula (15). Pyrrolo[3,4-b]pyridinones of general formula (15) may be treated with sodium hydride and alkylating agents such as iodomethane, allyl bromide, propargyl bromide or bromoacetonitrile to provide, following deprotection with TFA:CH₂Cl₂ (1:1) or 4N HCl in 1,4-dioxane, pyrrolo[3,4-b]pyridinones of general formula (11).

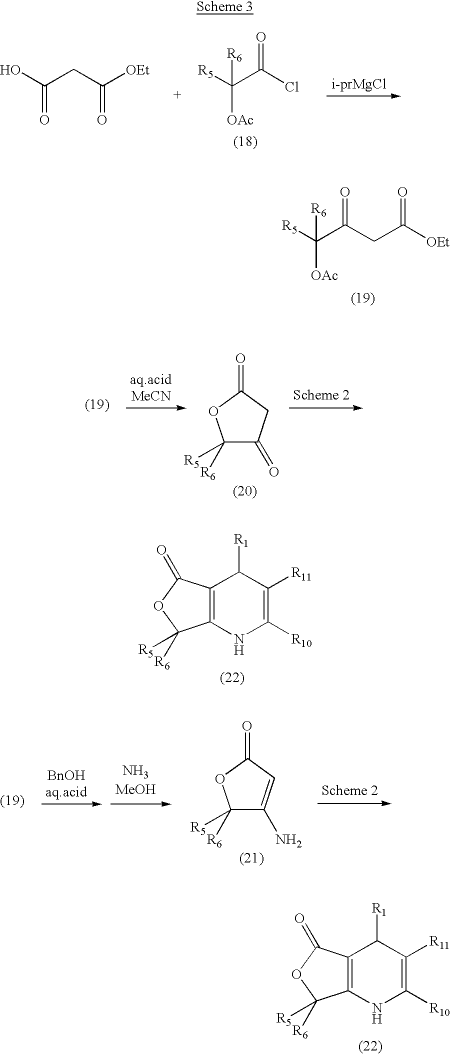

Furo[3,4-b]pyridinones of general formula (22), wherein R₁, R₅, R₆, R₁₀, and R₁₁ are as defined in formula I, may be prepared as described in Scheme 3 . 3-Ethoxy-3-oxopropanoic acid may be treated with isopropylmagnesium chloride and acid chlorides of general formula (18), wherein R₆ is hydrogen, to provide β-ketoesters of general formula (19). β-Ketoesters of general formula (19) may be treated with aqueous acid in acetonitrile to provide diones of general formula (20) as described in (Pollet and Gelin, Tetrahedron (1997) 34, 1453-1455). Diones of general formula (20) may be processed as described in Scheme 2 to provide furo[3,4-b]pyridinones of general formula (22), wherein R₆ is hydrogen. Alternatively, β-ketoesters of general formula (19) may be treated with aqueous acid in a solvent such as benzyl alcohol followed by treatment with ammonia/methanol to provide enaminones of general formula of (21). Enaminones of general formula (21) may then be processed as described in Scheme 2 to provide furo[3,4-b]pyridinones of general formula (22), wherein R₆ is hydrogen.

Diones of general formula (20), wherein R₅ and R₆ are independently selected from alkenyl, alkoxy, alkyl, alkynyl, cyanoalkyl, haloalkyl, or halogen, may be prepared as described in (Gelin and Pollet, Bull. Soc. Chimique, (1975) No. 1-2, 307-308). Disubstituted diones of general formula (20) may then be processed as described in Scheme 3 to provide furo[3,4-b]pyridinones of general formula (22) wherein R₅ and R₆ are independently selected from alkenyl, alkoxy, alkyl, alkynyl, cyanoalkyl, haloalkyl, or halogen.

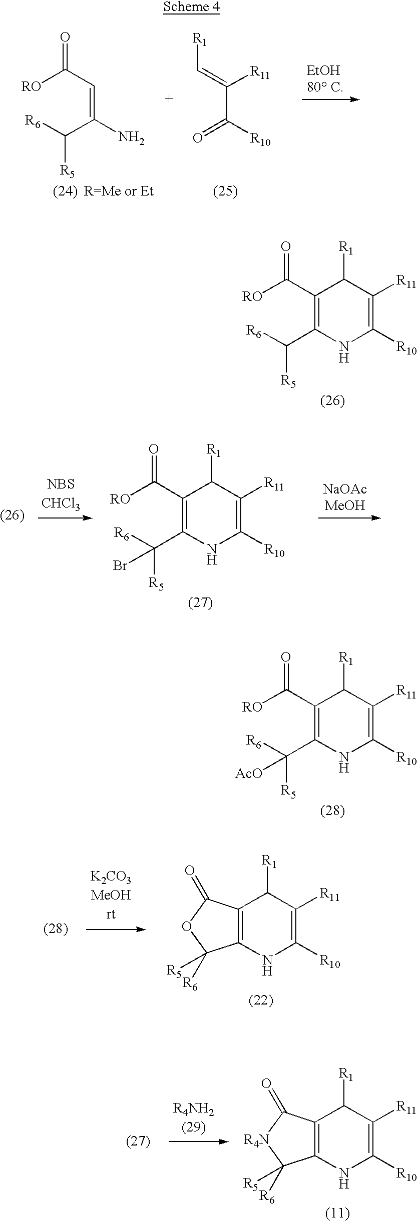

An alternative method for preparing furo[3,4-b]pyridinones of general formula (22), wherein R₁₀ is selected from aryl, arylalkyl, haloalkyl, heterocycle, heterocyclealkyl, hydroxyalkyl, and (NR₈R₉)alkyl and R₁, R₅, R₆, R₈, R₉, and R₁₁ are as defined in formula I, is described in Scheme 4. Enaminones of general formula (24), wherein R is lower alkyl such as methyl or ethyl, may be treated with α,β-unsaturated carbonyls of general formula (25) in ethanol at 80° C. to provide dihydropyridines of general formula (26). Dihydropyridines of general formula (26) may be treated with a brominating reagent such as pyridinium tribromide in pyridine/chloroform or NBS in a solvent such as methanol, ethanol, isopropanol or chloroform to provide bromoalkyl dihydropyridines of general formula (27). Bromoalkyl dihydropyridines of general formula (27) may be treated with sodium acetate in methanol to provide acetyloxy dihydropyridines of general formula (28). Acetyloxy dihydropyridines of general formula (28) may be treated with potassium carbonate in methanol at ambient temperature to provide furo[3,4-b]pyridinones of general formula (22). Alternatively, bromomethyl dihydropyridines of general formula (27) may be heated neat at 120-180° C. to provide with furo[3,4-b]pyridinones of general formula (22).

Pyrrolo[3,4-b]pyridinones of general formula (11), wherein R₁, R₄, R₅, R₆, R₁₀, and R₁₁ are as defined in formula I, may be prepared as described in Scheme 4. Bromoalkyl dihydropyridines of general formula (27) may be treated with a primary amine of general formula (29) in an alcoholic solvent to provide pyrrolo[3,4-b]pyridinones of general formula (11).

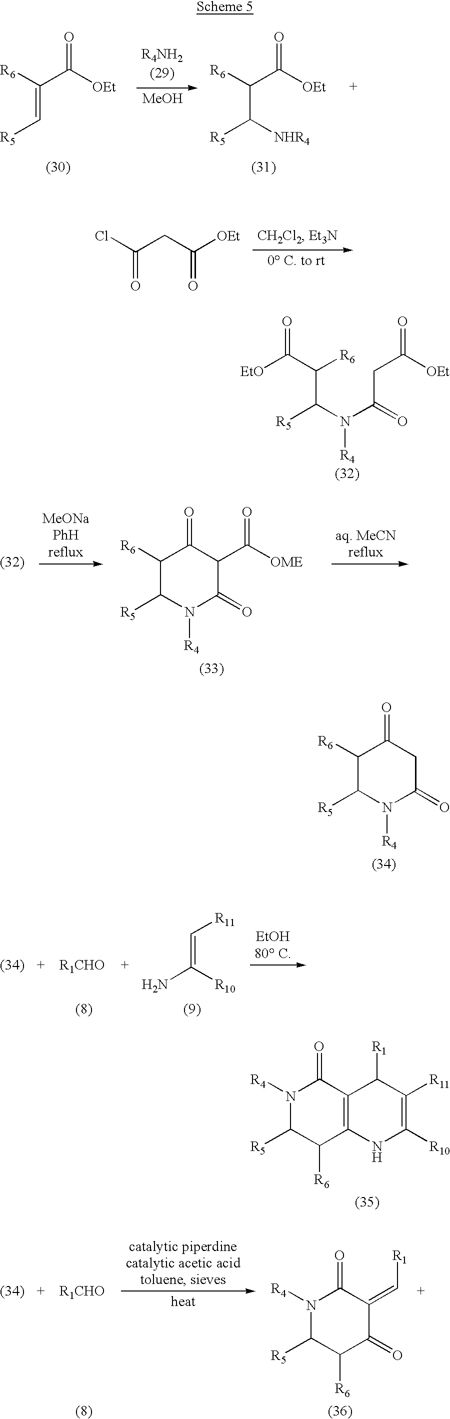

and R₁₁ are as defined in formula I, may be prepared as described in Scheme 5. α,β-Unsaturated esters of general formula (30) may be treated with primary amines of general formula (29) to provide aminoesters of general formula (31). Aminoesters of general formula (31) may be treated with ethyl 3-chloro-3-oxopropionate and triethylamine in methylene choride (0° C. to ambient temperature) to provide amides of general formula (32). Amides of general formula (32) may be treated with sodium methoxide in benzene at reflux to provide lactams of general formula (33). Lactams of general formula (33) may be treated with aqueous acetonitrile at reflux to provide diones of general formula (34) or diones of general formula (34) maybe prepared as described in (Micovic et al., J. Chem. Soc., Perkin Trans. (I) (1996)2041-2050). Diones of general formula (34) may be treated with aldehydes of general formula (8) and enamines of general formula (9) in ethanol at 80° C. to provide tetrahydro[1,6]naphthyridinones of general formula (35).

Alternatively, diones of general formula (34) may be treated with aldehydes of general formula (8), catalytic piperidine, pyrrolidine, or morpholine, catalytic amound of acid such as acetic acid and 4 Å molecular sieves in toluene with heat to provide α,β-unsaturated compounds of general formula (36). α,β-Unsaturated compounds of general formula (36) may be treated with enamines of general formula (9) in ethanol at 80° C. to provide tetrahydro[1,6]naphthyridinones of general formula (35).

Alternatively, diones of general formula (34) may be treated with ammonia in methanol and heat to provide enaminones of general formula (37). Diones of general formula (34) may also be treated in a stepwise fashion with ethanol and acid and then ammonia in methanol with heat to provide enaminones of general formula (37). Enaminones of general formula (37) may be treated with α,β-unsaturated compounds of general formula (25) in ethanol at 80° C. to provide tetrahydro[1,6]naphthyridinones of general formula (35).

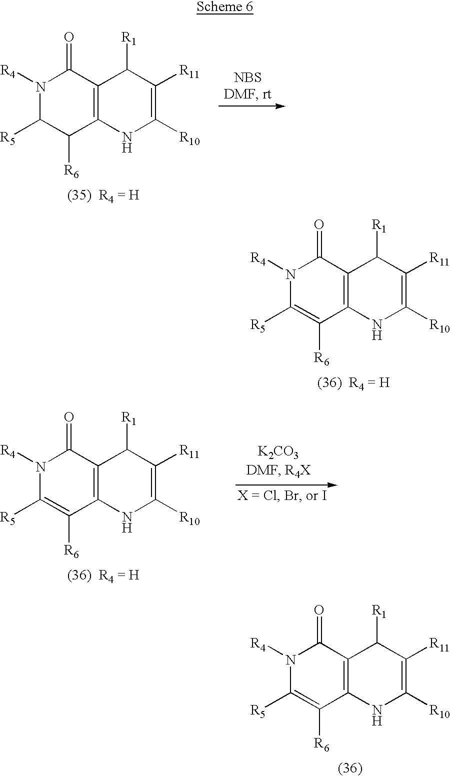

Dihydro[1,6]naphthyridinones of general formula (36), wherein R₁, R₄, R₅, R₆, R₁₀, and R₁₁ are as defined in formula I, may be prepared as described in Scheme 6. Tetrahydro[1,6]naphthyridinones of general formula (35), wherein R₄ is hydrogen, may be treated with N-bromosuccinimide at ambient temperature in DMF to provide dihydro[1,6]naphthyridinones of general formula (36) wherein R is hydrogen. Dihydro[1,6]naphthyridinones of general formula (36), wherein R₄ is hydrogen, may be treated with a base such as potassium carbonate and a halide such as iodomethane, allylbromide, propargyl bromide, or bromoacetonitrile in DMF to provide dihydro[1,6]naphthyridinones of general formula (36).

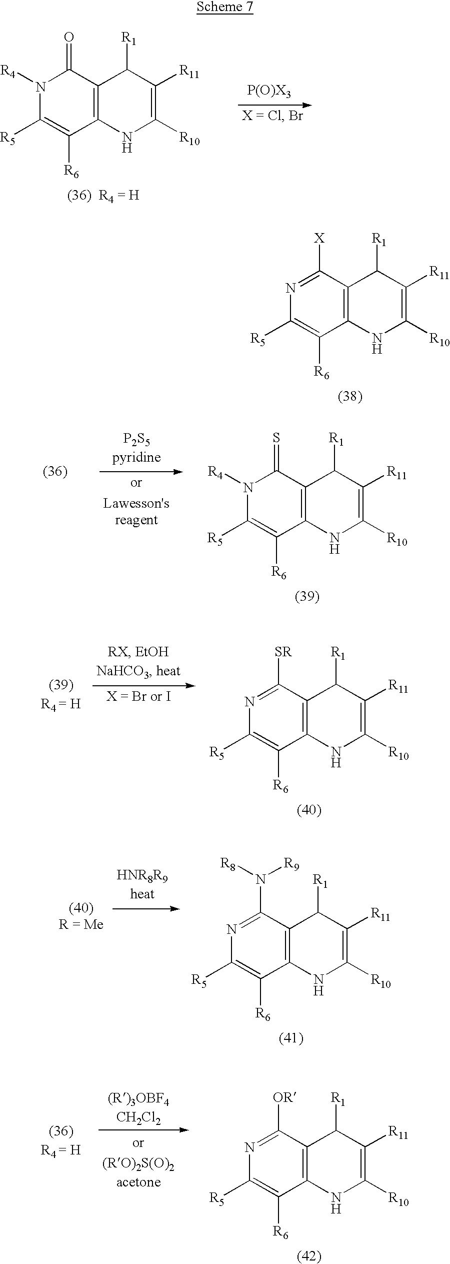

Dihydro[1,6]naphthyridines of general formulae (38), (40), (41) and (42) and dihydro[1,6]naphthyridinethiones of general formula (39), wherein R₁, R₄, R₅, R₆, R₈, R₉, R₁₀, and R₁₁ are as defined in formula I, may be prepared as described in Scheme 7. Dihydro[1,6]naphthyridinones of general formula (36), wherein R₄ is hydrogen, may be treated with phosphorous oxychloride or phosphorous oxybromide to provide dihydro[1,6]naphthyridines of general formula (38), wherein X is Cl or Br.

Dihydro[1,6]naphthyridinones of general formula (36) may be treated with phosphorous pentasulfide in pyridine or Lawesson's reagent to provide dihydro[1,6]naphthyridinethiones of general formula (39). Dihydro[1,6]naphthyridinethiones of general formula (39) wherein R₄ is hydrogen may be treated with a base such as sodium bicarbonate and an alkyl halide of general formula RX, wherein R is selected from alkenyl, alkyl, alkynyl, alkylcarbonyl, alkylcarbonylalkyl or cyanoalkyl, and X is preferably bromine or iodine, to provide dihydro[1,6]naphthyridines of general formula (40).

Dihydro[1,6]naphthyridines of general formula (40), wherein R is methyl, may be treated with amines and heat to provide dihydro[1,6]naphthyridines of general formula (41).

Dihydro[1,6]naphthyridinones of general formula (36) wherein R₄ is hydrogen may also be treated with oxonium tetrafluoroborates, wherein R′ is selected from alkenyl, alkyl, alkynyl, alkylcarbonyl, alkylcarbonylalkyl and cyanoalkyl, in methylene chloride or dihydro[1,6]naphthyridinones of general formula (36) wherein R₄ is hydrogen may be treated with sulfates, wherein R′ is selected from alkenyl, alkyl alkynyl, alkylcarbonyl, alkylcarbonylalkyl or cyanoalkyl, in acetone to provide dihydro[1,6]naphthyridines of general formula (42). Additional methods for O-alkylation of lactams is described in (Advances in Heterocyclic Chemistry, Glushkov and Granik, “The Chemistry of Lactim Ethers”, vol. 12, (1970) 185-212) and references cited therein, all icorporated by reference.

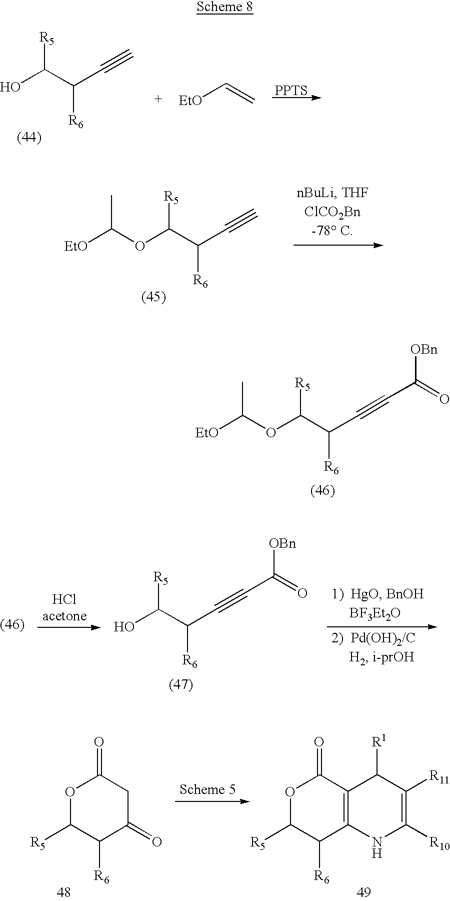

Tetrahydropyrano[4,3-b]pyridinones of general formula (49), wherein R₁, R₅, R₆, R₁₀, and R₁₁ are as defined in formula I, may be prepared as described in Scheme 8 or as described in (d'Angelo, J. and Gomez-Pardo, D., Tet. Letters, vol. 32, #26, (1991) 3063-3066). Alcohols of general formula (44) may be treated with 1-ethoxyethylene and a mild acid such as pyridinium p-toluenesulfonate to provide alkynes of general formula (45). Alkynes of general formula (45) may be treated with a strong base such as n-butyllithium and an acid chloride such as benzyl chloroformate to provide esters of general formula (46). Esters of general formula (46) may be treated with acid in acetone to provide alcohols of general formula (47). Alcohols of general formula (47) may be treated with mercury(II) oxide, boron trifluoride diethyl etherate and benzyl alcohol and then treated with a palladium catalyst such as palladium hydroxide on carbon under a hydrogen atmosphere in isopropanol to provide diones of general formula (48). Diones of general formula (48) may be processed as described in Scheme 5 to provide tetrahydropyrano[4,3-b]pyridinones of general formula (49).

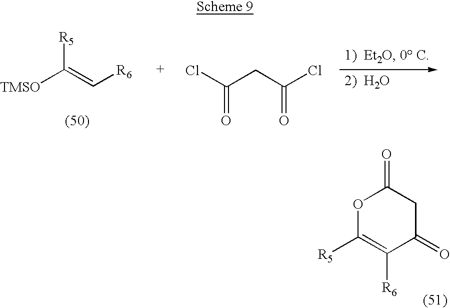

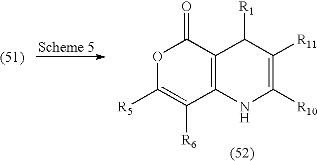

Dihydropyrano[4,3-b]pyridinones of general formula (52), wherein R₁, R₅, R₆, R₁₀, and R₁₁ are as defined in formula I, may be prepared as described in Scheme 9. Trimethylsilyl vinyl ethers of general formula (50) may be treated with malonyl dichloride in diethyl ether at 0° C. for 5 hours and then treated with water to provide diones of general formula (51). Diones of general formula (51) may also be prepared as described in (Effenberger et al., Chem. Ber. (1986) 119, 3394-3404; and Effenberger et al., Chem. Ber. (1985) 118, 741-752). Diones of general formula(51) may be processed as described in Scheme 5 to provide dihydropyrano[4,3-b]pyridinones of general formula (52).

The preparation of aldehydes used to synthesize many preferred compounds of the invention may be found in the following literature references: Pearson, Org. Synth. Coll. Vol V (1973), 117; Nwaukwa, Tetrahedron Lett. (1982), 23, 3131; Badder, J. Indian Chem. Soc. (1976), 53, 1053; Khanna, J. Med. Chem. (1997), 40, 1634; Rinkes, Recl. Trav. Chim. Pays-Bas (1945), 64, 205; van der Lee, Recl. Trav. Chim. Pays-Bas (1926),45, 687; Widman, Chem. Ber. (1882), 15, 167; Hodgson, J. Chem. Soc. (1927), 2425; Clark, J. Fluorine Chem. (1990), 50, 411; Hodgson, J. Chem. Soc. (1929), 1635; Duff, J. Chem. Soc. (1951), 1512; Crawford, J. Chem. Soc. (1956), 2155; Tanouchi, J. Med. Chem. (1981), 24, 1149; Bergmann, J. Am. Chem. Soc. (1959), 81, 5641; Other: Eistert, Chem. Ber. (1964), 97, 1470; Sekikawa, Bull. Chem. Soc. Jpn. (1959), 32, 551, all hereby incorporated by reference.

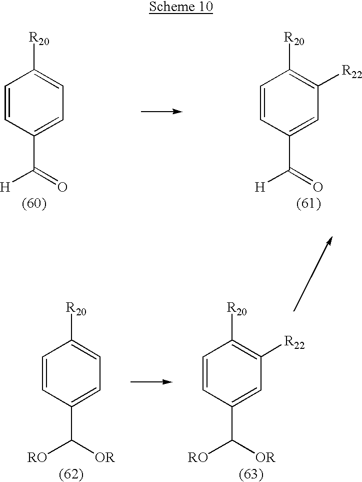

Meta, para-disubstituted aldehydes of general formula (61), wherein R₂₀ is selected from alkyl, haloalkyl, halo, haloalkoxy, alkoxy, alkylthio, —NR_{A}R_{B}, and —C(O)NR_{A}R_{B} wherein R_{A} and R_{B} are independently selected from hydrogen, alkyl, alkylcarbonyl, arylalkyl and formyl and R₂₂ is selected from nitro, halo, and alkylcarbonyl, may be prepared according to the method described in Scheme 10. A para substituted aldehyde of general formula (60) or the corresponding acetal protected aldehyde of general formula (62), wherein R is selected from alkyl or together with the oxygen atoms to which they are attached form a 5 or 6 membered ring wherein 1,3-dioxolanes are preferred, may by subjected to conditions of an electrophilic aromatic substitution reaction to provide aldehydes of general formula (61) or protected aldehydes of general formula (63). Preferred protecting groups for compounds of general formula (62) and (63) include dimethyl or diethyl acetals or the 1,3-dioxolanes. These protecting groups may be introduced at the beginning and removed at the end to provide substituted aldehydes of general formula (61) using methods well known to those skilled in the art of organic chemistry.

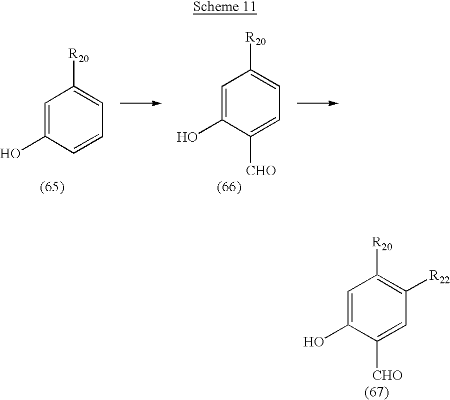

Aldehydes of general formula (67), wherein R₂₀ is selected from alkyl, haloalkyl, halo, haloalkoxy, alkoxy, alkylthio, —NR_{A}R_{B}, and —C(O)NR_{A}R_{B} wherein R_{A} and R_{B} are independently selected from hydrogen, alkyl alkylcarbonyl, arylalkyl and formyl and R₂₂ is selected from nitro, halo, and alkylcarbonyl, may be prepared by the method described in Scheme 11. A meta substituted phenol (65) is converted to the para substituted salicylaldehyde (66) by reaction with a base such as sodium hydroxide and a reagent such as trichloromethane or tribromomethane, known as the Reimer-Tiemann reaction. An alternate set of reaction conditions involves reaction with magnesium methoxide and paraformaldehyde as described in (Aldred, J. Chem. Soc. Perkin Trans. 1 (1994), 1823). The aldehyde (66) may be subjected to conditions of an electrophilic aromatic substitution reaction to provide meta, para disubstituted salicylaldehydes of general formula (67).

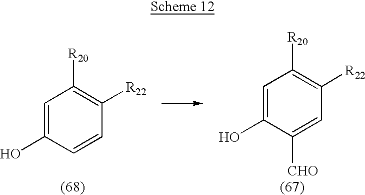

An alternative method of preparing meta, para disubstituted salicylaldehydes of general formula (67), wherein R₂₀ is selected from alkyl, haloalkyl, halo, haloalkoxy, alkoxy, alkylthio, —NR_{A}R_{B}, and —C(O)NR_{A}R_{B}, wherein R_{A} and R_{B} are independently selected from hydrogen, alkyl, alkylcarbonyl, arylalkyl, and formyl and R₂₂ is selected from nitro, halo, and alkylcarbonyl, may be used as described in Scheme 12. A meta, para disubstituted phenol of general formula (68) may be reacted with a base such as sodium hydroxide and a reagent such as trichloromethane or tribromomethane, known as the Reimer-Tiemann reaction, to provide disubstituted salicylaldehydes of general formula (67). An alternate set of reaction conditions involves reaction with magnesium methoxide and paraformaldehyde as described in (Aldred, J. Chem. Soc. Perkin Trans. 1 (1994), 1823).

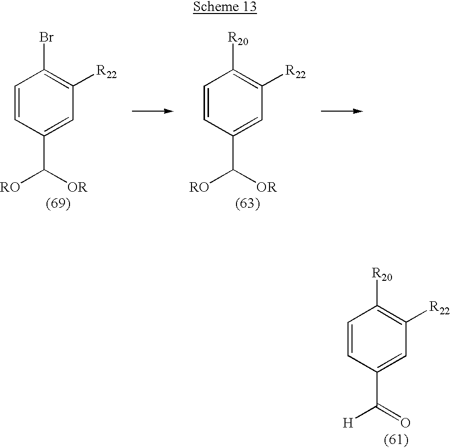

An alternative method of preparing benzaldehydes of general formula (61), wherein R₂₂ is selected from alkyl, haloalkyl, chlorine, fluorine, haloalkoxy, alkoxy, alkylthio, nitro, alkylcarbonyl, arylcarbonyl, —NR_{A}R_{B}, and —C(O)NR_{A}R_{B} wherein R_{A} and R_{B} are independently selected from hydrogen, alkyl, alkylcarbonyl, arylalkyl, and formyl, and R₂₀ is selected from alkyl, hydroxyalkyl, alkylthio, alkylcarbonyl, and formyl, is described in Scheme 13. Protected benzaldehydes of general formula (69), wherein R is selected from alkyl or together with the oxygen atoms to which they are attached form a 5 or 6 membered ring wherein 1,3-dioxolanes are preferred, may be converted to the 3,4-disubstituted benzaldehyde of general formula (63) via conversion of the bromide to an intermediate lithio or magnesio derivative, followed by reaction with an appropriate electrophile such as an aldehyde, dialkyldisulfide, a Weinreb amide, dimethylformamide, an alkyl halide or other electrophile followed by deprotection of the acetal to provide benzaldehydes of general formula (61).

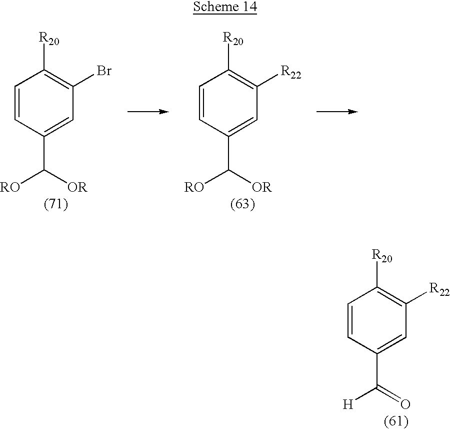

An alternative method of preparing benzaldehydes of general formula (61), wherein R₂₀ is selected from alkyl, haloalkyl, chlorine, fluorine, haloalkoxy, alkoxy, alkylthio, —NR_{A}R_{B}, and —C(O)NR_{A}R_{B} wherein R_{A} and R_{B} are independently selected from hydrogen, alkyl, alkylcarbonyl, arylalkyl, and formyl, R₂₂ is selected from alkyl, hydroxyalkyl, alkylthio, alkylcarbonyl, arylcarbonyl, and formyl, may be used as described in Scheme 14. Protected benzaldehydes of general formula (71), wherein R is selected from alkyl or together with the oxygen atoms to which they are attached form a 5 or 6 membered ring wherein 1,3-dioxolanes are preferred may be processed as described in Scheme 13 to provide benzaldehydes of general formula (61).

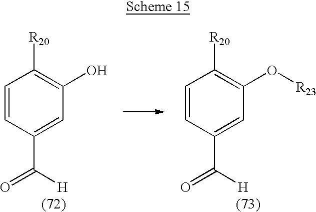

Benzaldehydes of general formula (73), wherein R₂₀ is selected from hydrogen, alkyl, alkylsulfonyl, aryl, heteroaryl, cyano, haloalkyl, halo, haloalkoxy, nitro, alkoxy, alkylthio, —NR_{A}R_{B}, and —C(O)NR_{A}R_{B} wherein R_{A} and R_{B} are independently selected from hydrogen, alkyl, alkylcarbonyl, arylalkyl, and formyl, and R₂₃ is selected from hydrogen, alkyl, arylalkyl, and haloalkyl wherein preferred haloalkyl groups are selected from difluoromethyl, 2,2,2-trifluoroethyl and bromodifluoromethyl, may be prepared as described in Scheme 15. 3-Hydroxybenzaldehydes of general formula (72) may be treated with suitable alkylating reagents such as benzylbromide, iodomethane, 2-iodo-1,1,1-trifluoroethane, chlorodifluoromethane, or dibromodifluoromethane in the presence of base such as potassium carbonate, potassium tert-butoxide or sodium tert-butoxide, to provide benzaldehydes of general formula (73). The synthesis of useful 3-hydroxybenzaldehydes of general formula (72) may be found in the following literature references: J. Chem. Soc. (1923), 2820; J. Med Chem. (1986), 29, 1982; Monatsh. Chem. (1963), 94, 1262; Justus Liebigs Ann. Chem. (1897), 294, 381; J. Chem. Soc. Perkin Trans. 1 (1990), 315; Tetrahedron Lett. (1990), 5495; J. Chem. Soc. Perkin Trans. 1 (1981), 2677.

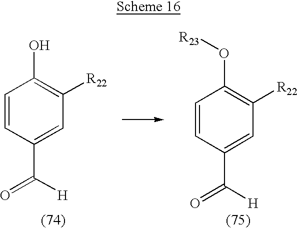

Benzaldehydes of general formula (75), wherein R₂₂ is selected from hydrogen, alkyl, alkylsulfonyl, aryl, heteroaryl, cyano, haloalkyl, halo, haloalkoxy, nitro, alkoxy, alkylthio, —NR_{A}R_{B}, and —C(O)NR_{A}R_{B} wherein R_{A} and R_{B} are independently selected from hydrogen, alkyl, alkylcarbonyl, arylalkyl, and formyl, and R₂₃ is selected from hydrogen, alkyl, arylalkyl, and haloalkyl wherein preferred haloalkyl groups are selected from difluoromethyl, 2,2,2-trifluoroethyl, and bromodifluoromethyl, may be prepared as described in Scheme 16. 4-Hydroxybenzaldehydes of general formula (74) may be treated with suitable alkylating reagents such as benzylbromide, iodomethane, 2-iodo-1,1,1-trifluoroethane, chlorodifluoromethane, or dibromodifluoromethane, in the presence of base such as potassium carbonate, potassium tert-butoxide or sodium tert-butoxide to provide benzaldehydes of general formula (75). The synthesis of useful 4-hydroxybenzaldehydes of general formula (74) may be found in the following literature references: Angyal, J. Chem. Soc. (1950),2141; Ginsburg, J. Am. Chem. Soc. (1951),73, 702; Claisen, Justus Liebigs Ann. Chem. (1913),401, 107; Nagao, Tetrahedron Lett. (1980),21,4931; Ferguson, J. Am. Chem. Soc. (1950),72, 4324; Barnes, J. Chem. Soc. (1950),2824; Villagomez-Ibarra, Tetrahedron (1995), 51, 9285; Komiyama, J. Am. Chem. Soc. (1983), 105, 2018; DE 87255; Hodgson, J. Chem. Soc. (1929), 469; Hodgson, J. Chem. Soc. (1929), 1641, all hereby incorporated by reference.

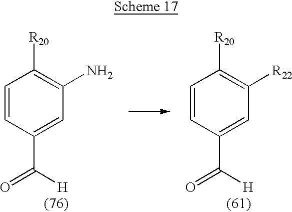

An alternate method for introduction of substituents at the 3-position of benzaldehydes of general formula (61), wherein R₂₀ is selected from hydrogen, alkyl, alkylsulfonyl, aryl, heteroaryl, cyano, haloalkyl, halo, haloalkoxy, nitro, alkoxy, alkylthio, and —C(O)NR_{A}R_{B}, wherein R_{A} and R_{B} are independently selected from hydrogen, alkyl, alkylcarbonyl, arylalkyl, and formyl may be used as described in Scheme 17. This method, also known as the Sandmeyer reaction, involves converting 3-amino benzaldehydes of general formula (76) to an intermediate diazonium salt with sodium nitrite. The diazonium salts may be treated with a bromine or iodine source to provide the bromide or iodide. The Sandmeyer reaction and conditions for effecting the transformation are well known to those skilled in the art of organic chemistry. The types of R₂₂ substituents that may be introduced in this fashion include cyano, hydroxy, or halo. In order to successfully carry out this transformation it may in certain circumstances be advantageous to perform the Sandmeyer reaction on a protected aldehyde. The resulting iodide or bromide may be treated with unsaturated halides, boronic acids or tin reagents in the presence of a palladium catalyst such as tetrakis(triphenylphosphine)palladium(O) to provide benzaldehydes of general formula (61). The diazonium salts may also be treated directly with unsaturated halides, boronic acids or tin reagents in the presence of a palladium catalyst such as tetrakis(triphenylphosphine)palladium (0) to provide benzaldehydes of general formula (61).

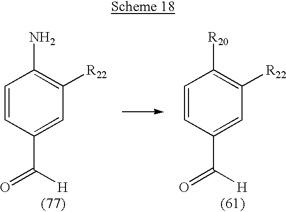

An alternate method for introduction of substituents at the 4-position of benzaldehydes of general formula (61), wherein R₂₂ is selected from hydrogen, alkyl, alkylsulfonyl, aryl, heteroaryl, cyano, haloalkyl, halo, haloalkoxy, nitro, alkoxy, alkylthio, and —C(O)NR_{A}R_{B}, wherein R_{A} and R_{B} are independently selected from hydrogen, alkyl, alkylcarbonyl, arylalkyl, and formyl, may be used as described in Scheme 18. This method, also known as the Sandmeyer reaction, involves converting 4-amino benzaldehydes of general formula (77) to an intermediate diazonium salt with sodium nitrite and then treating the diazonium salts in a similar manner as that described in Scheme 17. The types of R₂₀ substituents that may be introduced in this fashion include cyano, hydroxy, or halo. The Sandmeyer reaction and conditions for effecting the transformation are well known to those skilled in the art of organic chemistry. In order to successfully carry out this transformation it may in certain circumstances be advantageous to perform the Sandmeyer reaction on a protected aldehyde.

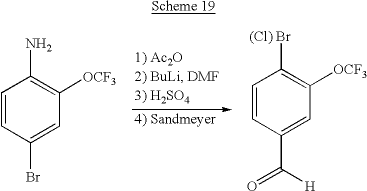

4-Bromo-3-(trifluoromethoxy)benzaldehyde or 4-chloro-3-(trifluoromethoxy)benzaldehyde may be prepared as described in Scheme 19. The commercially available 4-bromo-2-(trifluoromethoxy)aniline may be protected at the amino group with a suitable N-protecting group well known to those skilled in the art of organic chemistry such as acetyl or tert-butoxycarbonyl. The bromine may then be converted to the lithio or magnesio derivative and reacted directly with dimethylformamide to provide the 4-aminoprotected-3-(trifluoromethoxy)benzaldehyde derivative. Removal of the N-protecting group followed by conversion of the amine to a bromide or chloride via the Sandmeyer method of Scheme 18 provides 4-bromo-3-(trifluoromethoxy)benzaldehyde or 4-chloro-3-(trifluoromethoxy)benzaldehyde.

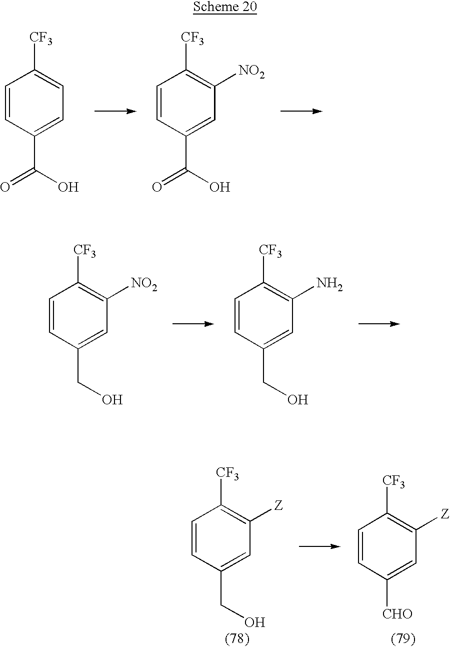

4-Trifluoromethylbenzaldehydes of general formula (79), wherein Z is selected from cyano, nitro, and halo may be prepared according to the method of Scheme 20. 4-Trifluoromethylbenzoic acid is first nitrated, using suitable conditions well known in the literature such as nitric acid with sulfuric acid, and the carboxylic acid group reduced with borane to provide 3-nitro-4-trifluoromethylbenzyl alcohol. From this benzyl alcohol may be obtained the 3-nitro-4-trifluoromethylbenzaldehyde by oxidation with typical reagents such as manganese dioxide. The nitro benzylic alcohol may be reduced to the aniline using any of a number of different conditions for effecting this transformation among which a preferred method is hydrogenation over a palladium catalyst. The aniline may be converted to either a halo or cyano substituent using the Sandmeyer reaction described in Scheme 17. Benzyl alcohols of general formula (78) may be oxidized using conditions well known to those skilled in the art such as manganese dioxide or Swern conditions to provide benzaldehydes of general formula (79).

For certain aromatic ring substitutions of R₁ for compounds of the present invention it is preferable to effect transformations of the aromatic ring substitutions after the aldehyde has been incorporated into the core structure of the present invention. As such, compounds of the present invention may be further transformed to other distinct compounds of the present invention. These transformations involve Stille, Suzuki and Heck coupling reactions all of which are well known to those skilled in the art of organic chemistry. Shown below are some representative methods of such transformations of compounds of the present invention to other compounds of the present invention.

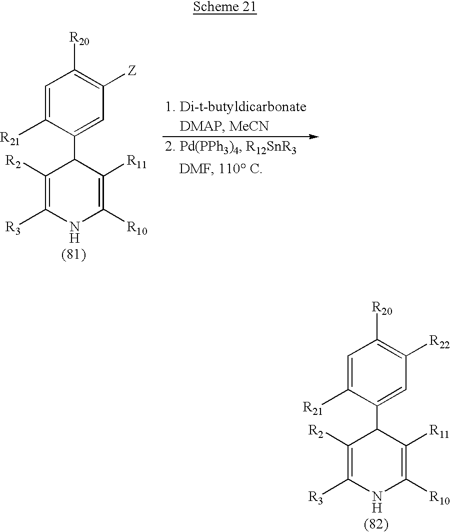

Dihydropyridines of general formula (82), wherein R₂, R₃, R₁₀, and R₁₁ are as defined in formula I, R₂₀ is selected from hydrogen, alkyl, alkylcarbonyl, alkylsulfonyl, cyano, haloalkyl, chlorine, fluorine, haloalkoxy, nitro, alkoxy, and alkylthio, and —C(O)NR_{A}R_{B} wherein R_{A} and R_{B} are independently selected from hydrogen, alkyl, alkylcarbonyl, arylalkyl, and formyl, R₂₁ is selected from hydrogen, hydroxy, alkoxy, haloalkoxy, and arylalkoxy, R₂₂ is selected from alkyl, vinyl, and cyano, maybe prepared as described in Scheme 21. Compounds of general formula (81), wherein Z is selected from bromine, iodine, and triflate, are protected with a tert-butoxycarbonyl (Boc) group using standard procedures. The aromatic bromide, iodide, or triflate may be treated with a suitable tin, boronic acid, or unsaturated halide reagent in the presence of a palladium catalyst with heating in a solvent such as dimethylformamide to effect a coupling reaction that provides dihydropyridines of general formula (82). The conditions for this transformation also effect the removal of the Boc protecting group.

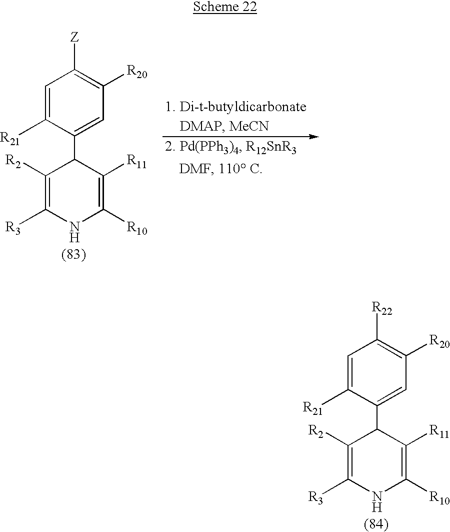

Dihydropyridines of general formula (84), wherein R₂, R₃, R₁₀, and R₁₁ are as defined in formula I, R₂₂ is selected from hydrogen, alkyl, alkylcarbonyl, alkylsulfonyl, cyano, haloalkyl, chlorine, fluorine, haloalkoxy, nitro, alkoxy, alkylthio, and —C(O)NR_{A}R_{B} wherein R_{A} and R_{B} are independently selected from hydrogen, alkyl, alkylcarbonyl, arylalkyl, and formyl, R₂₁ is selected from hydrogen, hydroxy, alkoxy, haloalkoxy, and arylalkoxy, R₂₀ is selected from alkyl, vinyl, aryl, and cyano, may be prepared as described in Scheme 22. Dihydropyridines of general formula (83), wherein Z is selected from bromine, iodine, and triflate, may be protected with a tert-butoxycarbonyl (Boc) group using standard procedures. The aromatic bromide, iodide, or triflate may be reacted with a suitable tin, boronic acid, or unsaturated halide reagent in the presence of a palladium catalyst with heating in a solvent such as dimethylformamide to effect a coupling reaction that provides dihydropyridines of general formula (84). The conditions for this transformation also effect the removal of the Boc protecting group.

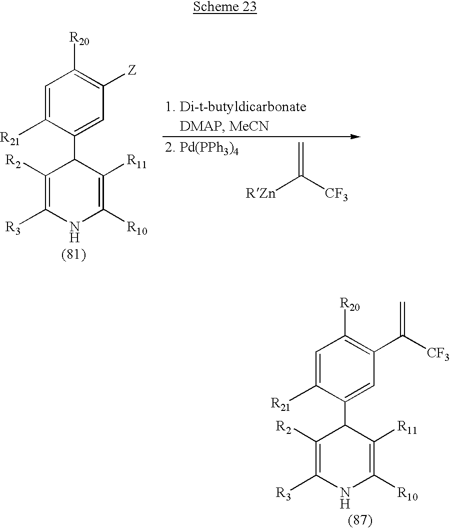

Dihydropyridines of general formula (87), wherein R₂, R₃, R₁₀, and R₁₁ are as defined in formula I, R₂₀ is selected from hydrogen, alkyl, alkylcarbonyl, alkylsulfonyl, cyano, haloalkyl, chlorine, fluorine, haloalkoxy, nitro, alkoxy, alkylthio, and —C(O)NR_{A}R_{B} wherein R_{A} and R_{B} are independently selected from hydrogen, alkyl, alkylcarbonyl, arylalkyl, and formyl, and R₂₁ is selected from hydrogen, hydroxy, alkoxy, haloalkoxy, and arylalkoxy, may be prepared as described in Scheme 23. Dihydropyridines of general formula (81), wherein Z is selected from bromine, iodine, and triflate may be protected with a tert-butoxycarbonyl (Boc) group using standard procedures. The aromatic bromide, iodide, or triflate may be treated with a suitable halozinc reagent in the presence of a palladium catalyst with heating in a solvent such as dimethylformamide to effect a coupling reaction that provides dihydropyridines of general formula (87). The conditions for this transformation also effect the removal of the Boc protecting group. The types of meta substituents that may be introduced in this fashion include trihalopropenyl and more specifically the trifluoropropenyl group.

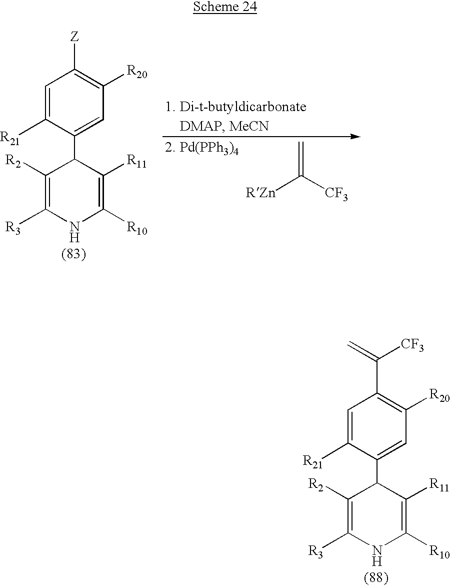

Dihydropyridines of general formula (88), wherein R₂, R₃, R₁₀, and R₁₁ are as defined in formula I, R₂₀ is selected from hydrogen, alkyl, alkylcarbonyl, alkylsulfonyl, cyano, haloalkyl, chlorine, fluorine, haloalkoxy, nitro, alkoxy, alkylthio, —C(O)NR_{A}R_{B} wherein R_{A} and R_{B} are independently selected from hydrogen, alkyl, alkylcarbonyl, arylalkyl, and formyl, R₂₁ is selected from hydrogen, hydroxy, alkoxy, haloalkoxy, and arylalkoxy, may be prepared as described in Scheme 24. Dihydropyridines of general formula (83), wherein Z is selected from bromine, iodine, and triflate may be protected with a tert-butoxycarbonyl (Boc) group using standard procedures. The aromatic bromide, iodide, or triflate may be treated with a suitable halozinc reagent in the presence of a palladium catalyst with heating in a solvent such as dimethylformamide to effect a coupling reaction that provides dihydropyridines of general formula (88). The conditions for this transformation also effect the removal of the Boc protecting group. The types of para substituents that may be introduced in this fashion include trihalopropenyl and more specifically the trifluoropropenyl group.

The compounds and processes of the present invention will be better understood by reference to the following examples, which are intended as an illustration of and not a limitation upon the scope of the invention. Further, all citations herein are incorporated by reference.

### EXAMPLE 1

### 4-(3-bromo-4-fluorophenyl)-3-cyano-2-methyl-4,6,7,8-tetrahydro[1,6]naphthyridin-5(1H)-one

### EXAMPLE 1A

### ethyl 3-oxo-3-[(3-ethoxy-3-oxopropyl)amino]propanoate

β-Alanine ethyl ester hydrochloride (1.54 g, 10.0 mmol) in dichloromethane (10 mL) was treated with triethylamine (1.54 mL, 11.0 mmol). After stirring at ambient temperature for 1 hour, the mixture was treated with additional triethylamine (1.54 mL, 11.0 mmol) and ethyl malonyl chloride (1.41 mL, 11.0 mmol) dropwise at 0° C. After stirring for 1 hour at 0° C. and 1 hour at ambient temperature, the mixture was treated with a 15% aqueous potassium carbonate solution (10 mL) and the layers were separated. The organic layer was washed with a 10% aqueous hydrochloric acid solution (10 mL), dried over magnesium sulfate, filtered and concentrated. The residue was purified by flash chromatography (silica, hexanes:ethyl acetate, 3:1 to 1:4) to provide the title compound as a colorless liquid (2.05 g, 89% yield).

MS (CI+) m/z 232 (M+H)⁺; ¹H NMR (CDCl₃) δ7.53 (br s, 1H), 4.24-4.14 (m, 4H), 3.57 (ddd, J=6.3, 6.3, 5.7 Hz, 2H), 3.30 (s, 2H), 2.56 (dd, J=6.0, 6.0 Hz, 2H), 1.28 (two dd, J=6.9, 3.3 Hz, 6H).

### EXAMPLE 1B

### methyl 2,4-dioxo-3-piperidinecarboxylate

Methanol (8.0 mL) was treated with sodium spheres (0.204 g, 8.87 mmol). After stirring at ambient temperature for 10 minutes, the product from Example 1A (2.05 g, 8.87 mmol) in dry benzene (50 mL) was added via cannula and the reaction mixture was refluxed for 5 hours. After cooling to ambient temperature, water was added, the layers were separated, and the organic layer was extracted with water (2×). The aqueous layers were combined and acidified with concentrated hydrochloric acid to pH 1. The acidified solution was extracted with dichloromethane:methanol (5:1) several times. The organic phases were combined, dried over magnesium sulfate, filtered and concentrated to provide the title compound (1.21 g, 80% yield).

MS (CI+) m/z 172 (M+H)⁺.

### EXAMPLE 1C

### 2,4-piperidinedione

The product from Example 1B (1.21 g, 7.08 mmol) was dissolved in a large volume of acetonitrile (1% water). After refluxing for 2 hours, solution was concentrated to provide the title compound as a yellow solid (quantitative yield).

MS (CI⁺) m/z 131 (M+NH₄)⁺; ¹H NMR (CDCl₃) δ6.64 (br s, 1H), 3.58 (ddd, J=6.3, 6.0, 3.6 Hz, 2H), 3.34 (s, 2H), 2.64 (dd, J=6.3, 6.3 Hz, 2H).

### EXAMPLE 1D

### 4-(3-bromo-4-fluorophenyl)-3-cyano-2-methyl-4,6,7,8-tetrahydro[1,6]naphthyridin-5(1H)-one

The product from Example 1C (0.520 g, 4.60 mmol), 3-bromo-4-fluorobenzaldehyde (0.934 g, 4.60 mmol) and 3-aminocrotononitrile (0.378 g, 4.60 mmol) in ethyl alcohol (25 mL) were stirred at 80° C. in a sealed tube for 12 hours. After cooling to ambient temperature, the reaction mixture was filtered to provide the title compound as a white solid (0.654 g). The filtrate was concentrated and flash chromatographed (silica, ethyl acetate:dichloromethane:methyl alcohol, 21:3:1 to 21:0:4) to provide an additional amount of the title compound (0.365 g, 61% combined yield).

MS (APCI+) m/z 362 (M+H)⁺; ¹H NMR (DMSO-d₆) δ9.33 (br s, 1H), 7.44 (dd, 1H, J=6.6, 2.1 Hz), 7.32 (dd, 1H, J=8.7, 8.7 Hz), 7.27-7.22 (m, 1H), 7.06 (br s, 1H), 4.53 (s, 1H), 3.21-3.16 (m, 2H), 2.55-2.33 (m, 2H), 2.05 (s, 3H); Anal. Calcd for C₁₆H₁₃BrFN₃O: C, 53.06; H, 3.62; N, 11.60. Found: C, 52.83; H, 3.44; N, 11.39.

### EXAMPLE 2

### 4-(3-bromo-4-fluorophenyl)-3-cyano-2-methyl-4,6,7,8-tetrahydro[1,6]naphthyridin-5(1H)-one

The racemic product from Example 1D was subjected to chiral HPLC chromatography ((R,R)-Whelk-O 1 column (2.1 cm×25 cm), 30% methanol:dichloromethane (2:1)/hexanes, flow rate 15 mL/minute) to provide the title compound as the less polar isomer.

[α]_{D}²³ −244° (c 0.005, DMSO); MS (APCI+) m/z 362 (M+H)⁺; ¹H NMR (DMSO-d₆) δ9.33 (br s, 1H), 7.44 (dd, 1H, J=6.6, 2.1 Hz), 7.32 (dd, 1H, J=8.7, 8.7 Hz), 7.27-7.22 (m, 1H), 7.06 (br s, 1H), 4.53 (s, 1H), 3.21-3.16 (m, 2H), 2.55-2.33 (m, 2H), 2.05 (s, 3H).

### EXAMPLE 3

### 4-(3-bromo-4-fluorophenyl)-3-cyano-2-methyl-4 6,7,8-tetrahydro[1,6]naphthyridin-5(1H)-one

The racemic product from Example 1D was subjected to chiral HPLC chromatography ((R,R)-Whelk-O 1 column (2.1 cm×25 cm), 30% methanol:dichloromethane (2:1)/hexanes, flow rate 15 mL/minute) to provide the title compound as the more polar isomer.

[α]_{D}²³ +244° (c 0.005, DMSO); MS (APCI+) m/z 362 (M+H)⁺,¹H NMR (DMSO-d₆) δ9.33 (br s, 1H), 7.44 (dd, 1H, J=6.6, 2.1 Hz), 7.32 (dd, 1H, J=8.7, 8.7 Hz), 7.27-7.22 (m, 1H), 7.06 (br s, 1H), 4.53 (s, 1H), 3.21-3.16 (m, 2H), 2.55-2.33 (m, 2H), 2.05 (s, 3H).

### EXAMPLE 4

### 4-(3-bromo-4-fluorophenyl)-3-cyano-2-methyl-4,6-dihydro[1,6]naphthyridin-5(1H)-one

The product from Example 1D (0.200 g, 0.55 mmol) in N,N-dimethylformamide (6 mL) was treated with N-bromosuccinimide (1.0 equiv, 98 mg) and stirred at ambient temperature for 3 hours. The mixture was concentrated and the residue was flash chromatographed (silica, ethyl acetate:dichloromethane:methyl alcohol, 21:3:1 to 21:3:3) to provide the title compound as a pale yellow solid (0.136 g, 68% yield).

MS (APCI+) m/z 360 (M+H)⁺; ¹H NMR (DMSO-d₆) δ11.07 (br s, 1H), 9.59 (s, 1H), 7.46 (dd, 1H, J=4.2, 1.5 Hz), 7.30 (dd, 1H, J=5.4, 5.4 Hz), 7.24 (ddd, 1H, J=5.1, 2.7, 1.2 Hz), 7.20 (d, 1H, J=4.2 Hz), 5.89 (d, 1H, J=4.2 Hz), 4.60 (s, 1H), 2.11 (s, 3H); Anal. Calcd for C₁₆H₁₁BrFN₃O 0.5 H₂O: C, 52.05; H, 3.28; N, 11.38. Found: C, 52.24; H, 3.23; N, 11.13.

### EXAMPLE 5

### 4-(3-bromo-4-fluorophenyl)-3-cyano-2-methyl-4,6-dihydro[1,6]naphthyridin-5(1H)-one

The racemic product from Example 4 was subjected to chiral BPLC chromatography ((R,R)-Whelk-O 1 column (2.1 cm×25 cm), 30% methanol:dichloromethane (2:1)/hexanes, flow rate 15 mL/minute) to provide the title compound as the less polar isomer, retention time 16 minutes.

MS (APCI+) m/z 360 (M+H)⁺; ¹H NMR (DMSO-d₆) δ11.07 (br s, 1H), 9.59 (s, 1H), 7.46 (dd, 1H, J=4.2, 1.5 Hz), 7.30 (dd, 1H, J=5.4, 5.4 Hz), 7.24 (ddd, 1H, J=5.1, 2.7, 1.2 Hz), 7.20 (d, 1H, J=4.2 Hz), 5.89 (d, 1H, J=4.2 Hz), 4.60 (s, 1H), 2.11 (s, 3H).

### EXAMPLE 6

### 4-(3-bromo-4-fluorophenyl)-3-cyano-2-methyl-4,6-dihydro[1,6]naphthyridin-5(1H)-one

The racemic product from Example 4 was subjected to chiral HPLC chromatography ((R,R)-Whelk-O 1 column (2.1 cm×25 cm), 30% methanol:dichloromethane (2:1)/hexanes, flow rate 15 mL/minute) to provide the title compound as the more polar isomer, retention time 30 minutes.

MS (APCI+) m/z 360 (M+H)⁺; ¹H NMR (DMSO-d₆) δ11.07 (br s, 1H), 9.59 (s, 1H), 7.46 (dd, 1H, J=4.2, 1.5 Hz), 7.30 (dd, 1H, J=5.4, 5.4 Hz), 7.24 (ddd, 1H, J=5.1, 2.7, 1.2 Hz), 7.20 (d, 1H, J=4.2 Hz), 5.89 (d, 1H, J=4.2 Hz), 4.60 (s, 1H), 2.11 (s, 3H).

### EXAMPLE 7

### 4-(3-bromo-4-fluorophenyl)-3-cyano-2,6-dimethyl-4,6-dihydro[1,6]naphthyridin-5(1H)-one

The product from Example 4 (0.309 g, 0.858 mmol) in DMF (7 mL) was treated succesively with potassium carbonate (2.0 equiv, 0.153 g) and iodomethane (25 equiv, 0.864 mL). The heterogeneous reaction mixture was stirred at ambient temperature for 72 hours, concentrated and the residue partitioned between water and dichloromethane. The layers were separated and the organic layer was dried over magnesium sulfate, filtered and concentrated. The residue was purified by flash chromatography (silica, dichloromethane:methanol, 30:1 to 10:1) to provide the title compound as a white solid (0.055 g, 17% yield).

MS (APCI+) m/z 374 (M+H)⁺; ¹H NMR (DMSO-d₆) δ9.61 (s, 1H), 7.51 (d, 1H, J=7.2 Hz), 7.46 (dd, 1H, J=6.9, 2.1 Hz), 7.30 (dd, 1H, J=8.4, 8.4 Hz), 7.23 (ddd, 1H, J=7.2, 5.1, 2.1 Hz), 5.92 (d, 1H, J=6.9 Hz), 4.62 (s, 1H), 3.27 (s, 3H), 2.11 (s, 3H).

### EXAMPLE 8

### 4-(3,4-dichlorophenyl)-3-cyano-2-methyl-4,6,7,8-tetahydro[1,6]naphthyridin-5(1H)-one

3,4-Dichlorobenzaldehyde was processed as described in Example 1D to provide the title compound.

MS (APCI+) m/z 334 (M+H)⁺; ¹H NMR (DMSO-d₆) δ2.02 (s, 3H), 2.38-2.56 (m, 2H), 3.20 (m, 2H), 4.58 (s, 1H), 7.01(s, 1H), 7.20-7.60 (m, 3H), 9.37 (s, 1H).

### EXAMPLE 9

### 4-(3-nitrophenyl)-3-cyano-2-methyl-4,6,7,8-tetrahydro[1,6]naphthyridin-5(1H)-one

3-Nitrobenzaldehyde was processed as described in Example 1D to provide the title compound.

MS (APCI+) m/z 311 (M+H)⁺; ¹H NMR (DMSO-d₆) δ2.02 (s, 3H), 2.38-2.60 (m, 2H), 3.20 (m, 2H), 4.70 (s, 1H), 7.01 (s, 11H), 7.60-8.10 (m, 4H), 9.38 (s, 1H).

### EXAMPLE 10

### 4-(4-chloro-3-nitrophenyl)-3-cyano-2-methyl-4,6,7,8-tetrahydro[1,6]naphthyridin-5(1H)-one

4-Chloro-3-nitrobenzaldehyde was processed as described in Example 1D to provide the title compound.

MS (APCI+) m/z 345 (M+H)⁺; ¹H NMR (DMSO-d₆) δ2.02 (s, 3H), 2.38-2.56 (m, 2H), 3.18 (m, 2H), 4.62 (s, 1H), 7.02 (s, 1H), 7.54-7.80 (m, 3H), 9.38 (s, 1H).

### EXAMPLE 11

### 4-(3,4-dibromophenyl)-3-cyano-2-methyl-4,6,7,8-tetrahydro[1,6]naphthyridin-5(1H)-one

3,4-Dibromobenzaldehyde was processed as described in Example 1D to provide the title compound.

MS (APCI+) m/z 421 (M+H)⁺; ¹H NMR (DMSO-d₆) δ2.02 (s, 3H), 2.36-2.56 (m, 2H), 3.20 (m, 2H), 4.56 (s, 1H), 7.01 (s, 1H), 7.16-7.64 (m, 3H), 9.30 (s, 1H).

### EXAMPLE 12

### 4-(3,4-difluorophenyl)-3-cyano-2-methyl-4,6,7,8-tetrahydro[1,6]naphthyridin-5(1H)-one

3,4-Difluorobenzaldehyde was processed as described in Example 1D to provide the title compound.

MS (APCI+) m/z 302 (M+H)⁺; ¹H NMR (DMSO-d₆) 62.02 (s, 3H), 2.35-2.50 (m, 2H), 3.18 (m, 2H), 4.50 (s, 1H), 6.98 (s, 1H), 7.00-7.35 (m, 3H), 9.22 (s, 1H).

### EXAMPLE 13

### 4-[4-fluoro-3-(trifluoromethyl)phenyl]-3-cyano-2-methyl-4,6,7,8-tetrahydro[1,6]naphthyridin-5(1H)-one

4-Fluoro-3-(trifluoromethyl)benzaldehyde was processed as described in Example 1D to provide the title compound.

MS (APCI+) m/z 352 (M+H)⁺; ¹H NMR (DMSO-d₆) δ2.01 (s, 3H), 2.36-2.56 (m, 2H), 3.20 (m, 2H), 4.62 (s, 1H), 7.00 (s, 1H), 7.40-7.60 (m, 3H), 9.30 (s, 1H).

### EXAMPLE 14

### 4-(2,4,5-trifluorophenyl)-3-cyano-2-methyl-4,6,7,8-tetrahydro[1,6]naphthyridin-5(1H)-one

2,4,5-Trifluorobenzaldehyde was processed as described in Example 1D to provide the title compound.

MS (APCI+) m/z 320 (M+H)⁺; ¹H NMR (DMSO-d₆) δ2.02 (s, 3H), 2.40-2.56 (m, 2H), 3.20 (m, 2H), 4.80 (s, 1H), 7.00 (s, 1H), 7.18-7.44 (m, 2H), 9.30 (s, 1H).

### EXAMPLE 15

### 4-(3-chloro-4-fluorophenyl)-3-cyano-2-methyl-4,6,7,8-tetrahydro[1,6]naphthyridin-5(1H)-one

3-Chloro-4-fluorobenzaldehyde was processed as described in Example 1D to provide the title compound.

MS (APCI+) m/z 318 (M+H)⁺; ¹H NMR (DMSO-d₆) δ2.01 (s, 3H), 2.36-2.52 (m, 2H), 3.20 (m, 2H), 4.58 (s, 1H), 6.99 (s, 1H), 7.20-7.38 (m, 3H), 9.24 (s, 1H).

### EXAMPLE 16

### 4-[4-chloro-3-(trifluoromethyl)phenyl]-3-cyano-2-methyl-4,6,7,8-tetrahydro[1,6]naphthyridin-5(1H)-one

4-Chloro-3-(trifluoromethyl)benzaldehyde was processed as described in Example 1D to provide the title compound.

MS (APCI+) m/z 368 (M+H)⁺; ¹H NMR (DMSO-d₆) δ 2.02 (s, 3H), 2.37-2.56 (m, 2H), 3.20 (m, 2H), 4.62 (s, 1H), 7.01(s, 1H), 7.52-7.62 (m, 3H), 9.36 (s, 1H).

### EXAMPLE 17

### 3-acetyl-4-(3-bromo-4-fluorophenyl)-2-methyl-4,6,7,8-tetrahydro[1,6]naphthyridin-5(1H)-one

The product from Example 1C (0.610 g, 5.40 mmol), 3-bromo-4-fluorobenzaldehyde (1.10 g, 5.40 mmol) and 2-amino-2-pentene-4-one (0.535 g, 5.40 mmol) in ethyl alcohol (25 mL) were stirred in a sealed tube at 80° C. for 12 hours. The reaction mixture was cooled to ambient temperature and filtered to provide the title compound as a white solid (2.05 g, 52% yield).

MS (APCI+) m/z 379 (M+H)⁺; ¹H NMR (DMSO-d₆) δ9.01 (s, 1H), 7.41 (dd, 1H, J=6.6, 1.8 Hz), 7.24 (dd, 1H, J=8.7, 8.7 Hz), 7.18 (ddd, 1H, J=7.2, 5.1, 2.1 Hz), 7.02 (d, 1H, J=1.8 Hz), 4.98 (s, 1H), 3.17-3.09 (m, 2H), 2.47-2.34 (m, 214),2.31 (s, 3H), 2.09 (s, 3H); Anal. Calcd for C₁₇H₁₆N₂O₂FBr: C, 53.84; H, 4.25; N, 7.39. Found: C, 53.74; H, 4.36; N, 7.50.

### EXAMPLE 18

### 3-acetyl-4-(3-bromo-4-fluorophenyl)-2-methyl-4,6-dihydro[1,6]naphthyridin-5(1H)-one

The product from Example 17 (0.100 g, 0.26 mmol) in N,N-dimethylformamide (2.6 mL) was treated with N-bromosuccinimide (1.0 equiv, 47 mg) and stirred at ambient temperature for 3 hours. The mixture was concentrated and the residue was flash chromatographed (silica, ethyl acetate:dichloromethane:methyl alcohol, 21:3:1 to 21:3:3) to provide the title compound as a pale yellow solid (0.050 g, 50.5% yield).

MS (APCI+) m/z 377 (M+H)⁺; ¹H NMR (DMSO-d₆) δ11.05 (br s, 1H), 9.30 (s, 1H), 7.54 (dd, 1H, J=6.6, 1.5 Hz), 7.26-7.20 (m, 2H), 7.15-7.13 (br m, 1H), 5.92 (d, 1H, J=7.2 Hz), 5.07 (s, 1H), 2.38 (s, 3H), 2.11 (s, 3H); Anal. Calcd for C₁₇H₁₄N₂O₂FBr: C, 54.13; H, 3.74; N, 7.43. Found: C, 53.96; H, 3.90; N, 7.31.

### EXAMPLE 19

### 3-acetyl-4-(3-bromo-4-fluorophenyl)-2-methyl-4,6-dihydro[1,6]naphthyridin-5(1H)-one

The racemic product from Example 18 was subjected to chiral HPLC chromatography ((R,R)-Whelk-O 1 column (2.1 cm×25 cm), 30% methanol:dichloromethane (2:1)/hexanes, flow rate 15 mL/minute) to provide the title compound as the less polar isomer, retention time 24 minutes.

MS (APCI+) m/z 377 (M+H)⁺; ¹H NMR (DMSO-d₆) δ11.05 (br s, 1H), 9.30 (s, 1H), 7.54 (dd, 1H, J=6.6, 1.5 Hz), 7.26-7.20 (m, 2H), 7.15-7.13 (br m, 1H), 5.92 (d, 1H, J=7.2 Hz), 5.07 (s, 1H), 2.38 (s, 3H), 2.11 (s, 3H).

### EXAMPLE 20

### 3-benzoyl-4-(3-bromo-4-fluorophenyl)-2-phenyl-4,6,7,8-tetrahydro[1,6]naphthyridin-5(1H)-one

### EXAMPLE 20A

### 2-(3-bromo-4-fluorobenzylidene)-1,3-diphenyl-1,3-propanedione

Dibenzoylmethane (0.224 g, 1.0 mmol) and 3-bromo-4-fluorobenzaldehyde (0.203 g, 1.0 mmol) in toluene was treated with catalytic amounts of piperidine (2 drops) and acetic acid (9 drops). After stirring in the presence of 4 Å molecular sieves at 85° C. for 12 hours, the mixture was allowed to cool to ambient temperature and then was filtered through a short pad of silica gel (hexanes:diethyl ether, 1:1). The filtrate was concentrated and the residue was purified by flash chromatography (silica, hexanes:diethyl ether, 6:1 to 4:1) to provide the title compound as a yellow foam (0.340 g, 83% yield).

¹H NMR (CDCl₃) δ7.96-7.93 (m, 2H), 7.88-7.85 (m, 2H), 7.63-7.40 (m, 8H), 7.28 (ddd, 1H, J=9.3, 5.1, 2.4 Hz), 6.97 (dd, 1H, J=8.4, 8.4 Hz).

### EXAMPLE 20B

### 3-benzoyl-4-(3-bromo-4-fluorophenyl)-2-phenyl-4,6,7,8-tetrahydro[1,6]naphthyridin-5(1H)-one

The product from Example 20A (0.340 g, 0.831 mmol) and the product from Example 1C (0.094 g, 0.831 mmol) in ethyl alcohol were treated with ammonium acetate (1.5 equiv, 0.096 g) and stirred in a sealed tube at 80° C. for 24 hours. The reaction mixture was allowed to cool to ambient temperature and concentrated. The residue was purified by flash chromatography (silica, dichloromethane:methyl alcohol, 8:0.3 to 8:0.5) to provide the title compound (0.120 g, 29% yield).

MS (APCI+) m/z 503 (M+H)⁺; ¹H NMR (DMSO-d₆) δ9.30 (s, 1H), 7.48 (dd, 1H, J=6.9, 1.8 Hz), 7.32-7.00 (m, 13H), 5.04 (s, 1H), 3.28-3.20 (m, 2H), 2.65-2.55 (m, 2H); Anal. Calcd for C₂₇H₂₀N₂O₂FBr.0.50 H₂O: C, 63.29; H, 4.13; N. 5.47. Found: C, 62.99; H, 4.37; N, 5.19.

### EXAMPLE 21

### 3-benzoyl-4-(3-bromo-4-fluorophenyl)-2-phenyl-4,6-dihydro[1,6]naphthyridin-5(1H)-one

The product from Example 20B (0.083 g, 0.165 mmol) in N,N-dimethylformamide was treated with N-bromosuccinimide (1.0 equiv, 29 mg) and stirred at ambient temperature for 3 hours. After concentration, the residue was purified by flash chromatography (silica, dichloromethane:methyl alcohol, 8:0.5) to provide the title compound as a yellow solid (0.054 g, 65% yield).

MS (APCI+) m/z 501 (M+H)⁺; ¹H NMR (DMSO-d₆) δ11.18 (d, 1H, J=5.4 Hz), 9.63 (s, 1H), 7.58 (dd, 1H, J=6.9, 2.1 Hz), 7.31-7.11 (m, 10H), 7.04-6.99 (m, 2H), 6.16 (d, 1H, J=6.9 Hz), 5.12 (s, 1H); Anal. Calcd for C₂₇H₁₈N₂O₂FBr.1.0 H₂O: C, 62.44; H, 3.88; N, 5.39. Found: C, 62.42; H. 4.14; N, 5.03.

### EXAMPLE 22

### 4-(3-bromo-4-fluorophenyl)-2-methyl-3-(3-methylbutanoyl)-4,6,7,8-tetrahydro[1,6]naphthyridin-5(1H)-one

### EXAMPLE 22A

### 3-(3-bromo-4-fluorobenzylidene)-6-methyl-2,4-heptanedione

6-Methyl-2,4-heptanedione (0.285 g, 2.0 mmol) and 3-bromo-4-fluorobenzaldehyde (0.406 g, 2.0 mmol) in toluene (10 mL) were treated with a catalytic amount of piperidine (4 drops) and acetic acid (15 drops). The reaction mixture was stirred in the presence of 4 Å molecular sieves at 80° C. for 12 hours. After cooling down to ambient temperature, the mixture was filtered through a short pad of silica gel (hexanes:ethyl acetate, 1:2). The filtrate was concentrated and the residue was purified by flash chromatography (silica, hexanes:ethyl acetate, 210:35 to 210:40) to provide the title compound as ayellow oil (0.345 g, 53% yield).

### EXAMPLE 22B

### 4-(3-bromo-4-fluorophenyl)-2-methyl-3-(3-methylbutanoyl)-4,6,7,8-tetrahydro[1,6]naphthyridin-5(1H)-one

The product from Example 22A (0.345 g, 1.055 mmol) and the product from Example 1C (0.119 g, 1.055 mmol) in ethyl alcohol were treated with ammonium acetate (1.5 equiv, 0.122 g) and stirred in a sealed tube at 80° C. for 72 hours. The reaction mixture was allowed to cool to ambient temperature and concentrated. The residue was purified by flash chromatography (silica, dichloromethane:ethyl acetate:methyl alcohol, 5:2:0.5) to provide the title compound (0.087 g, 20% yield).

MS (APCI+) m/z 421 (M+H)⁺; ¹H NMR (DMSO-d₆) δ8.92 (s, 1H), 7.39 (dd, 1H, J=5.1, 1.5 Hz), 7.23 (dd, 1H, J=6.6, 6.6 Hz), 7.18 (ddd, 1H, J=5.4, 3.6, 1.5 Hz), 6.97 (d, 1H, J=2.1 Hz), 5.02 (s, 1H), 3.20-3.06 (m, 2H), 2.48-2.31 (m, 2H), 2.41 (dd, 1H, J=11.7, 4.5 Hz), 2.27 (s, 3H), 2.10 (dd, 1H, J=11.7, 5.4 Hz), 1.95 (ddd, 1H, J=15.0, 10.2, 5.1 Hz), 0.79 (d, 3H, J=4.8 Hz), 0.69 (d, 3H, J=5.1 Hz); Anal. Calcd for C₂₀H₂₂FBrN₂O₂.0.25 H₂O: C, 56.41; H, 5.33; N, 6.58. Found: C, 56.40; H, 5.33; N, 6.42.

### EXAMPLE 23

### 4-(3-bromo-4-fluorophenyl)-2-methyl-3-(3-methylbutanoyl)-4,6-dihydro[1,6]naphthrdin-5(1H)-one

The product from Example 22B (0.077 g, 0.183 mmol) in N,N-dimethylformamide (2 mL) was treated with N-bromosuccinimide (1.0 equiv, 32.5 mg) and stirred at ambient temperature for 3 hours. After concentration, the residue was purified by flash chromatography (silica, dichloromethane:ethyl acetate:methyl alcohol, 5:2:0.5) to provide the title compound (0.040 g, 52% yield).

MS (APCI+) m/z 419 (M+H)⁺; ¹H NMR (DMSO-d₆) δ11.04 (br s, 1H), 9.24 (s, 1H), 7.53 (dd, 1H, J=5.1, 1.8 Hz), 7.26 (ddd, 1H, J=6.6, 4.2, 1.5 Hz), 7.22 (dd, 1H, J=6.6, 6.6 Hz), 7.13 (d, 1H, J=5.4 Hz), 5.91 (d, 1H, J=5.4 Hz), 5.10 (s, 1H), 2.45 (dd, 1H, J=12.0, 4.8 Hz), 2.35 (s, 3H), 2.14 (dd, 1H, J=12.0, 5.4 Hz), 1.96 (ddd, 1H, J=15.3, 10.2, 5.1 Hz), 0.81 (d, 3H, J=5.1 Hz), 0.70 (d, 3H, J=4.8 Hz); Anal. Calcd for C₂₀H₂₀N₂O₂FBr: C, 57.29; H, 4.81; N, 6.68. Found: C, 57.08; H, 4.92; N, 6.65.

### EXAMPLE 24

### 4-(3-bromo-4-fluorophenyl)-3-(2,2-dimethylpropanoyl)-2-(trifluoroyl)-4,6,7,8-tetrahydro[1,6]naphthyndin-5(1H)-one

### EXAMPLE 24A

### 3-(3-bromo-4-fluorobenzylidene)-1,1,1-trifluoro-5,5-dimethyl-2,4-hexanedione

1,1,1-Trifluoro-5,5-dimethyl-2,4-hexanedione (1.59 g, 8.08 mmol) and 3-bromo-4-fluorobenzaldehyde (1.64 g, 8.08 mmol) in toluene were treated with catalytic amounts of piperidine and acetic acid. After stirring in the presence of 4 Å molecular sieves at 85° C. for 12 hours, the mixture was allowed to cool to ambient temperature and then was filtered through a short pad of silica gel (hexanes:diethyl ether, 1:1). The filtrate was concentrated and the residue was purified by flash chromatography (silica, hexanes:diethyl ether, 9:1) to provide the title compound as a yellow foam (0.56 g, 18% yield).

### EXAMPLE 24B

### 4-(3-bromo-4-fluorophenyl)-3-(2,2-dimethylpropanoyl)-2-(trifluoromethyl)-4,6,7,8-tetrahydro[1,6]naphthyridin-5(1H)-one

The product from Example 24A (0.561 g, 1.47 mmol) and the product from Example 1C (0.166 g, 1.47 mmol) in ethyl alcohol were treated with ammonium acetate (2.0 equiv, 0.227 g) and stirred in a sealed tube at 80° C. for 72 hours. The reaction mixture was allowed to cool to ambient temperature and concentrated. The residue was purified by flash chromatography (silica, dichloromethane:methyl alcohol, 8:0.3 to 8:0.5) to provide the corresponding hemiaminal (63 mg).

The hemiaminal was suspended in toluene and treated with a catalytic amount of p-toluenesulphonic acid and refluxed overnight. The mixture was allowed to cool to ambient temperature and concentrated. The residue was purified by flash chromatography (silica, dichloromethane:ethyl acetate:methanol, 150:30:10) to provide the title compound as a white solid (10 mg).

MS (APCI)+ m/z 475 (M+H)⁺; ¹H NMR (DMSO-d₆) δ9.33 (br s, 1H), 7.32 (dd, 1H, J=6.8, 2.0 Hz), 7.30 (dd, 1H, J=8.8, 8.8 Hz), 7.14 (ddd, 1H, J=8.4, 4.8, 2.4 Hz), 7.08 (br s, 1H), 4.82 (s, 1H), 3.24-3.12 (m, 2H), 2.62-2.48 (m, 2H), 1.15 (s, 9H); Anal. Calcd for C₂₀H₁₉BrF₄N₂O₂: C, 50.54; H, 4.03; N, 5.89. Found: C, 50.55; H, 4.02; N, 5.70.

### EXAMPLE 25

### 4-[4-fluoro-3-(trifluoromethyl)phenyl]-3-cyano-2-methyl-1,4,6,7-tetrahydro-5H-pyrrolo[3,4-b]pyridin-5-one

### EXAMPLE 25A

### ethyl 3-[(2-ethoxy-2-oxoethyl)amino]-3-oxopropanoate

Glycine ethyl ester hydrochloride (21.07 g, 0.151 mole) in dichloromethane (450 mL) was treated with triethylamine (23.14 mL, 0.166 mole). After stirring at ambient temperature for 1 hour, the mixture was treated with additional triethylamine (23.14 mL, 0.166 mole) and ethyl malonyl chloride (25.0 g, 0.166 mole) dropwise at 0° C. After stirring for 1 hour at 0° C. and 1 hour at ambient temperature, the mixture was treated with a 15% aqueous potassium carbonate solution (450 mL) and the layers were separated. The organic layer was washed with a 10% aqueous hydrochloric acid solution (300 mL), dried over magnesium sulfate, filtered and concentrated. The residue (31.74 g, 97% yield) was used without further purification in the following step.

¹H NMR (CDCl₃) δ7.61 (br s, 1H), 4.27-4.18 (m, 4H), 4.07 (d, 2H, J=5.1 Hz), 3.37 (s, 2H), 1.33-1.26 (m, 6H).

### EXAMPLE 25B

### methyl 2,4-dioxo-3-pyrrolidinecarboxylate

Methanol (200 mL) was treated with sodium spheres (3.36 g, 0.146 mole). After stirring at ambient temperature for 1 hour, the product from Example 25A (31.74 g, 0.146 mole) in dry benzene (900 mL) was added via cannula and the reaction mixture was refluxed for 6 hours. After cooling to ambient temperature, water was added, the layers were separated, and the organic layer was extracted with water (2×). The aqueous layers were combined and acidified with concentrated hydrochloric acid to pH 1 to provide the title compound as a brown solid (14.5 g, 63.3% yield).

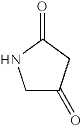

### EXAMPLE 25C

### 2,4-pyrrolidinedione

The product from Example 25B (1.50 g, 9.55 mmol) was dissolved in a large volume of acetonitrile (1% water). After refluxing for 3 hours, the solution was concentrated to provide the title compound as a yellow solid (0.94 g, quantitative yield). mixture of tautomers: ¹H NMR (DMSO-d₆) δ11.25 (s, 1H), 8.23 (br s, 1H), 7.07 (br s, 1H), 4.75 (m, 1H), 3.77 (m, 2H), 3.74 (s, 2H), 2.93 (t, J=1.2 Hz, 2H).

### EXAMPLE 25D

### 4-[4-fluoro-3-(trifluoromethyl)phenyl]-3-cyano-2-methyl-1,4,6,7-tetrahydro-5H-pyrrolo[3,4-b]pyridin-5-one

The product from Example 25C (0.568 g, 5.73 mmol), 4-fluoro-3-(trifluoromethyl)benzaldehyde (1.101 g, 5.73 mmol) and 3-aminocrotononitrile (0.470 g, 5.73 mmol) in ethyl alcohol (25 mL) were stirred at 80° C. in a sealed tube for 48 hours. After cooling to ambient temperature, the reaction mixture was concentrated and the residue purified by flash chromatography (silica, dichloromethane:methanol, 30:1 to 8:1) to provide the corresponding hemiaminal as a yellowish solid (0.707 g, 35% yield).

The hemiaminal (0.092 g, 0.259 mmol) was dissolved in methanol (2.5 mL) and treated with a hydrochloric acid solution (1.0M in diethyl ether, 2.0 mL). The reaction mixture was stirred at ambient temperature for 2 hours and then concentrated. The residue was purified by flash chromatography (silica, dichloromethane:methanol, 10:1) to provide the title compound as a white solid (0.064 g, 74% yield).

MS (APCI+) m/z 338 (M+H)⁺; ¹H NMR (DMSO-d₆) δ9.83 (s, 1H), 7.63-7.59 (m, 2H), 7.53-7.46 (m, 1H), 7.49 (br s, 1H), 4.69 (s, 1H), 3.94 (ABq, 2H, Δν=33.3 Hz, J=18.3 Hz), 2.09 (s, 3H); Anal. Calcd for C₁₆H₁₁F₄N₃O: C, 56.98; H, 3.29; N, 12.46. Found: C, 57.00; H, 3.28; N, 12.49.

### EXAMPLE 26

### 4-[4-fluoro-3-(trifluoromethyl)phenyl]-3-cyano-2-methyl-1,4,7,8-tetrahydro-5H-pyrano[4,3-b]pyridin-5-one

### EXAMPLE 26A

### 4-(1-ethoxyethoxy)-1-butyne

3-Butyn-1-ol (46.33 g, 0.661 mole) in methylene chloride (700 mL) was treated with ethyl vinyl ether (0.661 mole, 63.2 mL) and pyridinium p-toluenesulfonate (0.033, 8.31 g) (note: upon addition of pyridinium p-toluenesulfonate an exothermic reaction takes place). After stirring for a period of 2 hours, the reaction mixture was concentrated and filtered through a pad of silica gel (ethyl acetate:hexane, 1:1) to provide the title compound as a colorless liquid (80.29 g, 85.5% yield).

### EXAMPLE 26B

### benzyl 5-(1-ethoxyethoxy)-2-pentynoate

The product from Example 26A (79.99 g, 0.563 mole) in tetrahydrofuran (1 L) was treated dropwise at −78° C. with n-butyllithium (2.5M in hexanes, 0.563 mole, 225 mL). The reaction mixture was stirred at −78° C. for 30 minutes and then benzyl chloroformate (0.563 mole, 80.4 mL) was added dropwise. The reaction mixture was stirred at −78° C. for 2 hours, allowed to warm to ambient temperature and stirred overnight. After quenching with water, ethyl acetate was added and the layers were separated. The organic layer was dried over magnesium sulfate, filtered and concentrated. The residue was purified by flash chromatography (silica, hexane to hexane:ethyl acetate, 30:1 to 4:1) provide the title compound as a colorless oil (155.5 g, 78% yield).

### EXAMPLE 26C

### benzyl 5--hydroxy-2-pentynoate

The product from Example 26B (122.1 g, 0.442 mole) in acetone (400 mL) was treated at ambient temperature with an aqueous hydrochloric acid solution (0.5N, 200 mL). The reaction mixture was stirred for 6 hours and then diluted with water and ethyl acetate. The layers were separated, and the organic layer was dried over magnesium sulfate, filtered and concentrated to provide the title compound as a colorless oil (90.17 g, 100% yield).

¹H NMR (CDCl₃) δ2.61 (t, 2H), 3.79 (t, 2H), 5.19 (s, 2H), 7.32-7.40 (m, 5H).

### EXAMPLE 26D

### 4-(benzyloxy)-5,6-dihydro-2H-pyran-2-one

A heterogeneous mixture of benzyl alcohol (2.65 mole, 274.4 mL), mercury (II) oxide (red) (13.26 mmol, 2.87 g) and boron trifluoride diethyl etherate (0.133 mole, 16.3 mL) were heated at 60° C. for 3 hours (eventually turned homogeneous). The mixture was treated with the product from Example 26C (90.17 g, 0.442 mole) in benzyl alcohol (91.5 mL) at ambient temperature. After stirring at 70° C. for 4 hours, the mixture was allowed to cool to ambient temperature and stirred overnight. The reaction mixture was poured into an aqueous saturated sodium bicarbonate solution and extracted with ethyl acetate. The organic layer was dried over magnesium sulfate, filtered and concentrated. The residue was purified by flash chromatography (silica, hexane to hexane:ethyl acetate, 30:1 to 1:2) to provide the title compound as a white solid (49.6 g, 55% yield).

¹H NMR (CDCl₃) δ2.60 (t, 2H), 4.38 (t, 2H), 4.95 (s, 2H), 5.28 (s, 1H), 7.32-7.46 (m, 5H).

### EXAMPLE 26E

### dihydro-2H-pyran-2,4(3H)-dione

The product from Example 26D (9.17 g, 0.045 mole) in isopropanol (500 mL) was treated with palladium hydroxide (20 wt. % palladium, dry basis, on carbon) (4 g) under a nitrogen atmosphere. The reaction mixture was stirred under a hydrogen atmosphere at atmospheric pressure overnight and then filtered through a pad of silica gel (elution with ethyl acetate). The filtrate was concentrated to provide the title compound as a white solid (4.28 g, 84%).

¹H NMR (CDCl₃) δ2.73 (t, 2H), 3.57 (s, 2H), 4.61 (t, 2H).

### EXAMPLE 26F

### 4-[4-fluoro-3-(trifluoromethyl)phenyl]-3-cyano-2-methyl-1,4,7,8-tetrahydro-5H-pyrano[4,3-b]pyridin-5-one

The product from Example 26E (0.171 g, 1.5 mmol), 4-fluoro-3-(trifluoromethyl)benzaldehyde (0.288 g, 1.5 mmol) and 3-aminocrotononitrile (0.123 g, 1.5 mmol) in ethyl alcohol (5 mL) were stirred at 80° C. in a sealed tube for 48 hours. After cooling to ambient temperature, the precipitate (desired racemic product) was collected by filtration (0.101 g). The filtrate was concentrated and flash chromatographed (silica, ethyl acetate:dichloromethane:methanol, 7:1:0 to 7:1:0.1) to provide an additional amount of the title compound (0.083 g, 34.8% combined yield).

The racemic product was subjected to chiral HPLC chromatography ((R,R)-Whelk-O 1 column (2.1 cm×25 cm), 10% to 30% methanol:dichloromethane (2:1)/hexanes, flow rate 15 mL/minutes) to provide the title compound as the less polar isomer, retention time 42 minutes.

MS APCI(+) m/z 353 (M+H)⁺; ¹H NMR (DMSO-d₆) δ9.78 (s, 1H), 7.64 (ddd, 1H, J=7.5, 4.8, 2.1 Hz), 7.57 (dd, 1H, J=6.9, 2.4 Hz), 7.48 (dd, 1H, J=10.8, 10.8 Hz), 4.63 (s, 1H), 4.33-4.17 (m, 2H), 2.71 (ddd, 1H, J=16.5, 10.2, 5.4 Hz), 2.55 (dt, 1H, J=17.4, 4.5, 4.5 Hz), 2.07 (s, 3H).

### EXAMPLE 27

### 4-[4-fluoro-3-(trifluoromethyl)phenyl]-3-cyano-2-methyl-1,4,7,8-tetrahydro-5H-pyrano[4,3-b]pyridin-5-one

The racemic product from Example 26F was subjected to chiral HPLC chromatography ((R,R)-Whelk-O 1 column (2.1 cm×25 cm), 10% to 30% methanol:dichloromethane (2:1)/hexanes, flow rate 15 mL/minutes) to provide the title compound as the more polar isomer, retention time 50 minutes.

MS APCI(+) m/z 353 (M+H)⁺; ¹H NMR (DMSO-d₆) δ9.78 (s, 1H), 7.64 (ddd, 1H, J=7.5, 4.8, 2.1 Hz), 7.57 (dd, 1H, J=6.9, 2.4 Hz), 7.48 (dd, 1H, J=10.8, 10.8 Hz), 4.63 (s, 1H), 4.33-4.17 (m, 2H), 2.71 (ddd, 1H, J=16.5, 10.2, 5.4 Hz), 2.55 (dt, 1H, J=17.4, 4.5, 4.5 Hz), 2.07 (s, 3H).

### EXAMPLE 28

### 4-(3-bromo-4-fluorophenyl)-3-cyano-2-methyl-1,4,7,8-tetrahydro-5H-pyrano[4,3-b]pyridin-5-one

The product from Example 26E (0.171 g, 1.5 mmol), 3-bromo-4-fluorobenzaldehyde (0.305 g, 1.5 mmol) and 3-aminocrotononitrile (0.123 g, 1.5 mmol) in ethyl alcohol (5 mL) were stirred at 80° C. in a sealed tube for 48 hours. After cooling to ambient temperature, the reaction mixture was concentrated and the residue was purified by flash chromatography (silica, ethyl acetate:dichloromethane:methanol, 7:1:0 to 7:1:0.1) to provide the title compound as a white solid (0.140 g, 25.7% yield).

MS APCI(+) m/z 363 (M+H)⁺; ¹H NMR (DMSO-d₆) δ9.76 (s, 1H), 7.51 (dd, 1H, J=7.2, 2.4 Hz), 7.34 (dd, 1H, J=8.4, 8.4 Hz), 7.30 (ddd, I H, J=8.4, 4.8, 1.8 Hz), 4.51 (s, 1H), 4.33-4.18 (m, 2H), 2.71 (ddd, 1H, J=17.1, 10.8, 5.7 Hz), 2.55 (dt, 1H, J=13.2, 4.5, 4.5 Hz), 2.07 (s, 3H); Anal. Calcd for C₁₆H₁₂BrFN₂O₂: C, 52.91; H, 3.33; N, 7.71. Found: C, 52.58; H, 3.68; N, 7.98.

### EXAMPLE 29

### 4-(3-bromo-4-fluorophenyl)-3-methoxycarbonyl-2-methyl-1,4,7,8-tetrahydro-5H-pyrano[4,3-b]pyridin-5-one

The product from Example 26E (0.171 g, 1.5 mmol), 3-bromo-4-fluorobenzaldehyde (0.305 g, 1.5 mmol) and methyl-3-aminocrotonate (0.173 g, 1.5 mmol) in ethyl alcohol (5 mL) were stirred at 80° C. in a sealed tube for 48 hours. After cooling to ambient temperature, the precipitate (desired product) was collected by filtration (0.120 g). The filtrate was concentrated and flash chromatographed (silica, ethyl acetate:dichloromethane:methanol, 7:1:0 to 7:1:0.1) to provide an additional amount of the title compound (0.182 g, 51% combined yield).

MS APCI(+) m/z 396 (M+H)⁺; ¹H NMR (DMSO-d₆) δ9.42 (s, 1H), 7.36 (dd, 1H, J=6.6, 2.1 Hz), 7.25 (dd, 1H, J=8.4, 8.4 Hz), 7.19 (ddd, 1H, J=6.6, 5.4, 2.4 Hz), 4.82 (s, 1H), 4.28 (ddd, 1H, J=11.4, 5.7, 4.8 Hz), 4.13 (ddd, 1H, J=1.1, 11.1, 4.8 Hz), 2.69 (ddd, 1H, J=17.4, 11.1, 5.7 Hz), 2.55 (dt, 1H, J=17.7, 4.5, 4.5 Hz), 2.30 (s, 3H); Anal. Calcd for C₁₇H₁₅BrFNO₄: C, 51.53; H, 3.82; N, 3.54. Found: C, 51.39; H, 3.70; N, 3.47.

### EXAMPLE 30

### 4-[4-fluoro-3-(trifluoromethyl)phenyl]-3-methoxycarbonyl-2-methyl-1,4,7,8-tetrahydro-5H-pyrano[4,3-b]pyridin-5-one

4-Fluoro-3-(trifluoromethyl)benzaldehyde was processed as described in Example 29 to provide the title compound as a white solid (0.212 g, 36.7% yield).

MS APCI(+) m/z 386 (M+H)⁺; ¹H NMR (DMSO-d₆) δ9.46 (s, 1H), 7.54-7.48 (m, 1H), 7.46-7.36 (m, 2H), 4.88 (s, 1H), 4.32-4.25 (m, 1H), 4.14 (ddd, 1H, J=10.8, 10.8, 4.2 Hz), 2.70 (ddd, 1H, J=17.1, 11.1, 5.7 Hz), 2.55 (dt, 1H, J=17.7, 4.5, 4.5 Hz), 2.30 (s, 3H); Anal. Calcd for C₁₈H₁₅F₄NO₄: C, 56.11; H, 3.92; N, 3.64. Found: C, 55.95; H, 3.84; N, 3.56.

### EXAMPLE 31

### 4-(3-bromo-4-fluorophenyl)-5-chloro-3-(methoxycarbonyl-2-methyl-1,4-dihydro[1,6]naphthyridine

### EXAMPLE 31A

### 4-(3-bromo-4-fluorophenyl)-3-(methoxycarbonyl)-2-methyl-4,6,7,8-tetrahydro[1,6]naphthyridin-5(1H)-one

The product from Example 1C (1.33 g, 11.8 mmol), 3-bromo-4-fluorobenzaldehyde (2.39 g, 11.8 mmol) and methyl-3-aninocrotonate (1.36 g, 11.8 mmol) in ethyl alcohol (30 mL) were stirred in a sealed tube at 75° C. for 24 hours. The reaction mixture was allowed to cool to ambient temperature and filtered to provide the title compound as a white solid (2.27 g). The filtrate was concentrated and flash chromatographed (silica, ethyl acetate:dichloromethane:methyl alcohol, 4:1:0 to 7:0:1) to provide an additional amount of the title compound (0.54 g, 60% combined yield).

mp 150-152° C.; MS (ESI+) m/z 395 (M+H)⁺; ¹H NMR (DMSO-d₆) δ8.99 (s, 1H), 7.36 (dd, 1H, J=6.9, 2.1 Hz), 7.22 (dd, 1H, J=8.4, 8.4 Hz), 7.16 (ddd, 1H, J=8.4, 5.1, 2.1 Hz), 6.96 (br s, 1H), 4.90 (s, 1H), 3.52 (s, 3H), 3.19-3.11 (m, 2H), 2.45-2.33 (m, 2H), 2.29 (s, 3H); Anal. Calcd for C₁₇H₁₆N₂O₃FBr: C, 51.66; H, 4.08; N, 7.09. Found: C, 51.82; H, 4.37; N, 6.90.

### EXAMPLE 31B

### 4-(3-bromo-4-fluorophenyl)-3-(methoxycarbonyl)-2-methyl-4,6-dihydro[1,6]naphthyridin-5(1H)-one

The product from Example 31A (2.26 g, 5.72 mmol) in N,N-dimethylformamide (27 mL) was treated with N-bromosuccinimide (1.0 equiv, 1.02 g) and stirred at ambient temperature for 3 hours. After concentration, the residue was purified by flash chromatography (silica, dichloromethane:methyl alcohol, 30:1 to 10:1) to provide the title compound (1.47 g, 65% yield).

MS (APCI+) m/z 393 (M+H)⁺; ¹H NMR (DMSO-d₆) δ10.99 (br s, 1H), 9.29 (s, 1H), 7.45 (dd, 1H, J=4.5, 0.9 Hz), 7.22-7.19 (m, 2H), 7.14 (d, 1H, J=4.2 Hz), 5.92 (d, 1H, J=4.2 Hz), 4.99 (s, 1H), 3.54 (s, 3H), 2.36 (s, 3H); Anal. Calcd for C₁₇H₁₄N₂O₃FBr: C, 51.93; H. 3.59; N, 7.12. Found: C, 51.63; H, 3.65; N, 6.93.

### EXAMPLE 31C

### 4-(3-bromo-4-fluorophenyl)-5-chloro-3-(methoxycarbonyl)-2-methyl-1,4-dihydro[1,6]naphthyridine

The product from Example 31B (0.200 g, 0.509 mmol) was treated with a large excess of phosphorus oxychloride (4 mL) and stirred under a nitrogen atmosphere at 105° C. for 12 hours. After cooling to ambient temperature, the reaction mixture was poured dropwise into an ice-water solution, treated with potassium carbonate and extracted with dichloromethane. The organic layer was dried over magnesium sulfate, filtered, and concentrated. The residue was purified by flash chromatography (silica, ethyl acetate:dichloromethane:methanol, 5:1:0.2 to 5:1:0.5 to provide the title compound as a yellow solid (0.041 g, 20% yield).

MS (APCI+) m/z 411 (M+H)⁺; ¹H NMR (DMSO-d₆) δ9.86 (br s, 1H), 8.06 (d, 1H, J=5.1 Hz), 7.39 (dd, 1H, J=6.6, 2.1 Hz), 7.26 (dd, 1H, J=8.7, 8.7 Hz), 7.19 (ddd, 1H, J=8.4, 5.1, 2.1 Hz), 6.95 (d, 1H, J=5.7 Hz), 5.17 (s, 1H), 3.61 (s, 3H), 2.36 (s, 3H); Anal. Calcd for C₁₇H₁₃N₂O₂BrClF: C, 49.60; H, 3.18; N, 6.80. Found: C, 49.75; H, 3.19; N, 6.57.

### EXAMPLE 32

### 4-(3-bromo-4-fluorophenyl)-5-chloro-3-cyano-2-methyl-1,4-dihydro[1,6]naphthyridine

The product from Example 4 (0.500 g, 1.39 mmol) was treated with phosphorous oxychloride (19 mL). After refluxing overnight, the mixture was allowed to cool to ambient temperature, poured dropwise into an ice-water solution, treated with potassium carbonate (till ph=7-8) and extracted with dichloromethane. The organic layer was dried over magnesium sulfate, filtered, and concentrated. The residue was purified by flash chromatography (silica, ethyl acetate:hexanes, 4:1) to provide the title compound as an amorphous powder (0.351 g, 67% yield).

MS (APCI+) m/z 378 (M+H)⁺; ¹H NMR (DMSO-d₆) δ10.15 (br s, 1H), 8.10 (d, 1H, J=5.7 Hz), 7.46 (dd, 1H, J=6.9, 2.4 Hz), 7.36 (dd, 1H, J=8.7, 8.7 Hz), 7.18 (ddd, 1H, J=8.4, 4.8, 2.4 Hz), 6.92 (d, 1H, J=5.4 Hz), 4.93 (s, 1H), 2.12 (s, 3H); Anal. Calcd for C₁₆H₁₀N₃BrClF: C, 50.76; H, 2.66; N, 11.10. Found: C, 50.66; H, 2.69; N, 10.93.

### EXAMPLE 33

### 4-(3-bromo-4-fluorophenyl)-5-chloro-3-cyano-2-methyl-1,4-dihydro[1,6]naphthyridine

The racemic product from Example 32 was subjected to chiral HPLC chromatography ((R,R)-Whelk-O 1 column (2.1 cm×25 cm), 13% ethanol/hexanes, flow rate 15 mL/minute) to provide the title compound as the less polar isomer, retention time 33 minutes.

MS (APCI+) m/z 378 (M+H)⁺, ¹H NMR (DMSO-d₆) δ10.15 (br s, 1H), 8.10 (d, 1H, J=5.7 Hz), 7.46 (dd, 1H, J=6.9, 2.4 Hz), 7.36 (dd, 1H, J=8.7, 8.7 Hz), 7.18 (ddd, 1H, J=8.4, 4.8, 2.4 Hz), 6.92 (d, 1H, J=5.4 Hz), 4.93 (s, 1H), 2.12 (s, 3H).

### EXAMPLE 34

### 4-(3-bromo-4-fluorophenyl)-5-chloro-3-cyano-2-methyl-1,4-dihydro[1,6]naphthyridine

The racemic product from Example 32 was subjected to chiral HPLC chromatography ((R,R)-Whelk-O 1 column (2.1 cm×25 cm), 13% ethanol/hexanes, flow rate 15 mL/minute) to provide the title compound as the more polar isomer, retention time 38 minutes.

MS (APCI+) m/z 378 (M+H)⁺; ¹H NMR (DMSO-d₆) δ10.15 (br s, 1H), 8.10 (d, 1H, J=5.7 Hz), 7.46 (dd, 1H, J=6.9, 2.4 Hz), 7.36 (dd, 1H, J=8.7, 8.7 Hz), 7.18 (ddd, 1H, J=8.4, 4.8, 2.4 Hz), 6.92 (d, 1H, J=5.4 Hz), 4.93 (s, 1H), 2.12 (s, 3H).

### EXAMPLE 35

### 4-(3-bromo-4-fluorophenyl)-3-cyano-2-methyl-4,6-dihydro[1,6]naphthyridine-5(1H)-thione

The product from Example 4 (1.17 g, 3.25 mmol) was suspended in pyridine (30 mL) and treated with phosphorous pentasulfide (1.0 equiv, 1.44 g). After stirring at reflux for 36 hours, the mixture was allowed to cool to ambient temperature, poured onto an ice-water solution and extracted with dichloromethane:methanol (5:1). The layers were separated and the organic layer was dried over magnesium sulfate, filtered and concentrated. The residue was purified by flash chromatography (silica, dichloromethane:methanol, 20:1) to provide the title compound as a light brown solid (0.132 g, 11% yield).

MS (APCI+) m/z 376 (M+H)⁺; ¹H NMR (DMSO-d₆) δ12.77 (br s, 1H), 9.97 (s, 1H), 7.51 (dd, 1H, J=4.8, 4.8 Hz), 7.46 (d, 1H, J=3.9 Hz), 7.29 (dd, 1H, J=6.3, 6.3 Hz), 7.23 (br s, 1H), 6.42 (d, 1H, J=5.1 Hz), 5.01 (s, 1H), 2.13 (s, 3H); Anal. Calcd for C₁₆H₁₁N₃SFBr: C, 51.08; H, 2.95; N, 11.17. Found: C, 50.96; H, 3.05; N, 10.93.

### EXAMPLE 36

### 4-(3-bromo-4-fluorophenyl)-3-cyano-2-methyl-5-[(2-oxobutylsulfanyl]-1,4-dihydro[1,6]naphthyridine

The product from Example 35 (0.050 g, 0.133 mmol) in ethanol (5 mL) was treated succesively with sodium acetate (1.5 equiv, 0.027 g) and 1-bromo-2-butanone (90%, 0.177 mmol, 0.018 mL). After refluxing for 3 hours, the mixture was allowed to cool to ambient temperature and concentrated. The residue was purified by flash chromatography (silica, dichloromethane:methanol, 40:1 to 30:1) to provide the title compound as a crystalline yellow/brown solid (0.045 g, 76% yield).

MS (APCI+) m/z 446 (M+H)⁺; ¹H NMR (DMSO-d₆) δ9.90 (s, 1H), 8.08 (d, 1H, J=3.3 Hz), 7.41 (dd, 1H, J=3.9, 1.2 Hz), 7.34 (dd, 1H, J=5.4, 5.4 Hz), 7.17 (ddd, 1H, J=5.4, 3.0, 1.8 Hz), 6.68 (d, 1H, J=3.3 Hz), 4.76 (s, 1H), 3.94 (ABq, 2H, Δν=30.9 Hz, J=9.9 Hz), 2.46 (q, 2H, J=4.2 Hz), 2.10 (s, 3H), 0.88 (t, 3H, J=4.5 Hz); Anal. Calcd for C₂₀H₁₇N₃OSFBr: C, 53.82; H, 3.84; N, 9.41. Found: C, 53.91; H, 3.82; N, 9.20.

### EXAMPLE 37

### 4-(3-bromo-4-fluorophenyl)-3-cyano-2-methyl-5-(methylsulfanyl)-1,4-dihydro[1,6]naphthyridine

The product from Example 35 (0.033 g, 0.088 mmol) in ethanol (3 mL) was treated succesively with sodium acetate (1.5 equiv, 0.018 g) and iodomethane (0.105 mmol, 0.007 mL). After stirring at reflux for 2 hours, the mixture was allowed to cool to ambient temperature and concentrated. The residue was purified by flash chromatography (silica, dichloromethane:methanol, 40:1) to provide the title compound as a crystalline light brown solid (0.025 g, 73.5% yield).

MS (APCI+) m/z 390 (M+H)⁺; ¹H NMR (DMSO-d₆) δ9.87 (s, 1H), 8.18 (d, 1H, J=4.2 Hz), 7.39 (dd, 1H, J=5.1, 1.8 Hz), 7.33 (dd, 1H, J=6.3, 6.3 Hz), 7.15 (ddd, 1H, J=6.3, 3.6, 1.5 Hz), 6.69 (d, 1H, J=4.2 Hz), 4.71 (s, 1H), 2.37 (s, 3H), 2.09 (s, 3H); Anal. Calcd for C₁₇H₁₃N₃SBrF: C, 52.32; H, 3.36; N, 10.77. Found: C, 52.35; H, 3.40; N, 10.60.

### EXAMPLE 38

### 4-(3-bromo-4-fluorophenyl)-3-cyano-2-methyl-5-methoxy-1,4-dihydro[1,6]naphthyridine

The product from Example 4 (0.300 g, 0.833 mmol) suspended in dichloromethane (20 mL) was treated with sodium carbonate (20 equiv, 1.77 g) at 0° C. After stirring for 10 minutes, the mixture was treated with trimethyloxonium tetrafluoroborate (5.0 equiv, 0.616 g). After warning to ambient temperature and stirring for 12 hours, the mixture was poured into water. The layers were separated and the organic layer was dried over magnesium sulfate, filtered and concentrated. The residue was purified by flash chromatography (silica, dichloromethane:methanol, 40:1 to 15:1) to provide the title compound as a yellow solid (0.074g, 24% yield).

MS (APCI+) m/z 374 (M+H)⁺; ¹H NMR (DMSO-d₆) δ9.80 (s, 1H) 7.87 (d, 1H, J=5.4 Hz), 7.41 (dd, 1H, J=6.9, 2.4 Hz), 7.31 (dd, 1H, J=9.0, 9.0 Hz), 7.19 (ddd, 1H, J=9.0, 5.1, 2.4 Hz), 6.54 (d, 1H, J=5.7 Hz), 4.77 (s, 1H), 3.69 (s, 3H), 2.12 (s, 3H); Anal. Calcd for C₁₇H₁₃N₃OBrF: C, 54.56; H, 3.50; N, 11.23. Found: C, 54.26; H, 3.63; N, 11.09.

### EXAMPLE 39

### 4-(3-bromo-4-fluorophenyl)-5-chloro-3-(ethoxycarbonyl)-2-methyl-1,4-dihydro[1,6]naphthyridine

### EXAMPLE 39A

### 4-(3-bromo-4-fluorophenyl)-3-(ethoxacarbonyl)-2-methyl-4,6,7,8-tetrahydro[1,6]naphthyridin-5(1H)-one

The product from Example 1C (10 mmol, 1.13 g), 3-bromo-4-fluorobenzaldehyde (10 mmol, 2.03 g) and ethyl-3-aminocrotonate (10 mmol, 1.29 g) in ethanol (20 mL) were stirred in a sealed tube at 80° C. for 72 hours. The reaction mixture was allowed to cool to ambient temperature and concentrated. The residue was purified by flash chromatography (silica, dichloromethane:methanol, 30:1 to 10:1) to provide the title compound as a yellow solid (2.56 g, 63% yield).

¹H NMR (DMSO-d₆) δ8.97 (s, 1H), 7.38 (dd, 1H, J=6.9, 2.1 Hz), 7.23 (dd, 1H, J=8.4, 8.4 Hz), 7.17 (ddd, 1H, J=8.7, 5.4, 2.1 Hz), 6.97 (br s, 1H),4.88 (s, 1H), 4.07-3.89 (m, 2H), 3.19-3.12 (m, 2H), 2.54-2.33 (m, 2H), 2.28 (s, 3H), 1.11 (t, 3H, J=6.9 Hz).

### EXAMPLE 39B

### 4-(3-bromo-4-fluorophenyl-3-(ethoxycarbonyl)-2-methyl-4,6-dihydro[1,6]naphthyridin-5(1H)-one

The product from Example 39A (1.28 g, 3.13 mmol) in N,N-dimethylformamide (10 mL) was treated with N-bromosuccinimide (1.0 equiv, 0.557 g). The reaction mixture was stirred at ambient temperature for 3 hours and concentrated. The residue was purified by flash chromatography (silica, dichloromethane:methanol, 16:1) to provide the title compound as a pale yellow solid (0.99 g, 78% yield).

MS (APCI+) m/z 407 (M+H)⁺; ¹H NMR (DMSO-d₆) δ11.03 (br s, 1H), 9.28 (s, 1H), 7.45 (dd, 1H, J=7.5, 2.1 Hz), 7.22-7.12 (m, 3H), 5.92 (d, 1H, J=6.9 Hz), 4.96 (s, 1H), 4.03-3.93 (m, 2H), 2.35 (s, 3H), 1.13 (t, 3H, J=7.2 Hz); Anal. Calcd for C₁₈H₁₆BrFN₂O₃.0.40 C₃H₇NO: C, 00; H, 00; N, 00. Found: C, 52.45; H, 4.06; N, 7.42.

### EXAMPLE 39C

### 4-(3-bromo-4-fluorophenyl)-5-chloro-3-(ethoxycarbonyl)-2-methyl-1,4-dihydro[1,6]naphthyridine

The product from Example 39B (0.505 g, 1.24 mmol) was treated with phosphorous oxychloride (10 mL) and stirred at 115° C. for 12 hours. The reaction mixture was allowed to cool to ambient temperature and poured dropwise into an ice-water solution. Solid potassium carbonate was added in small portions, followed by extraction with dichloromethane:methanol (10:1). The organic layer was dried over magnesium sulfate, filtered and concentrated. The residue was purifieds by flash chromatography (silica, dichloromethane:methanol, 20:1) to provide the title compound as a brown solid (0.100 g, 19% yield).

MS (APCI+) m/z 425 (M+H)⁺; ¹H NMR (DMSO-d₆) δ9.83 (s, 1H), 8.05 (d, 1H, J=4.2 Hz), 7.42 (dd, 1H, J=5.1, 1.5 Hz), 7.25 (dd, 1H, J=6.6, 6.6 Hz), 7.17 (ddd, 1H, J=6.6, 3.9, 1.8 Hz), 6.95 (d, 1H, J=4.2 Hz), 5.16 (s, 1H), 4.14-3.98 (m, 2H), 2.36 (s, 3H), 1.20 (t, 3H, J=5.4 Hz).

### EXAMPLE 40

### 4-(3-bromo-4-fluorophenyl)-5-chloro-3-(ethoxycarbonyl)-2-methyl-1,4-dihydro[1,6]naphthyridine

The racemic product from Example 39C was subjected to chiral HPLC chromatography ((R,R)-Whelk-O 1 column (2.1 cm×25 cm), 13% ethanol/hexanes, flow rate 15 mL/minute) to provide the title compound as the less polar isomer, retention time 19 minutes.

MS (APCI+) m/z 425 (M+H)⁺; ¹H NMR (DMSO-d₆) δ9.83 (s, 1H), 8.05 (d, 1H, J=4.2 Hz), 7.42 (dd, 1H, J=5.1, 1.5 Hz), 7.25 (dd, 1H, J=6.6, 6.6 Hz), 7.17 (ddd, 1H, J=6.6, 3.9, 1.8 Hz), 6.95 (d, 1H, J=4.2 Hz), 5.16 (s, 1H), 4.14-3.98 (m, 2H), 2.36 (s, 3H), 1.20 (t, 3H, J=5.4 Hz).

### EXAMPLE 41

### 4-(3-bromo-4-fluorophenyl)-5-chloro-3-(ethoxycarbonyl-2-methyl-1,4-dihydro[1,6]naphthyridine

The racemic product from Example 39C was subjected to chiral HPLC chromatography ((R,R)-Whelk-O 1 column (2.1 cm×25 cm), 13% ethanol/hexanes, flow rate 15 mL/minute) to provide the title compound as the more polar isomer, retention time 22 minutes.

MS (APCI+) m/z 425 (M+H)⁺; ¹H NMR (DMSO-d₆) δ9.83 (s, 1H), 8.05 (d, 1H, J=4.2 Hz), 7.42 (dd, 1H, J=5.1, 1.5 Hz), 7.25 (dd, 1H, J=6.6, 6.6 Hz), 7.17 (ddd, 1H, J=6.6, 3.9, 1.8 Hz), 6.95 (d, 1H, J=4.2 Hz), 5.16 (s, 1H), 4.14-3.98 (m, 2H), 2.36 (s, 3H), 1.20 (t, 3H, J=5.4 Hz).

### EXAMPLE 42

### 4-(3-bromo-4-fluorophenyl)-3-cyano-6-cyanomethyl-2-methyl-4,6-dihydro[1,6]naphthyridin-5(1H)-one

The product from Example 4 (0.302 g, 0.838 mmol) in DMF (12 mL) was treated succesively with potassium carbonate (2.0 equiv, 0.232 g) and bromoacetonitrile (20 equivalents, 1.17 mL). The heterogeneous reaction mixture was stirred at ambient temperature for 6 hours. Following concentration, the residue was purified by flash chromatography (silica, dichloromethane:methanol, 30:1) to provide the title compound as a grey solid (0.163 g, 48.7% yield).

MS (APCI+) m/z 399 (M+H)⁺; ¹H NMR (DMSO-d₆) δ9.77 (s, 1H), 7.60 (d, 1H, J=7.5 Hz), 7.49 (dd, 1H, J=6.9, 2.1 Hz), 7.31 (dd, 1H, J=8.7, 8.7 Hz), 7.26 (ddd, 1H, J=9.3, 6.0, 3.0 Hz), 6.05 (d, 1H, J=7.5 Hz), 4.86 (ABq, 2H, Δν=17.4 Hz, J=18.3 Hz), 4.67 (s, 1H), 2.12 (s, 3H).

### EXAMPLE 43

### 4-(3-bromo-4-fluorophenyl)-6-(cyanomethyl)-3-(methoxycarbonyl)-2-methyl-4,6-dihydro[1,6]naphthyridin-5(1H)-one

The product from Example 31B (0.103 g, 0.262 mmol) in DMF (2.6 mL) was treated succesively with potassium carbonate (2.0 equiv, 0.072 g) and bromoacetonitrile (20 equiv, 0.36 mL). After stirring at ambient temperature for 2 hours, the heterogeneous reaction mixture was concentrationed and the residue was purified by flash chromatography (silica, dichloromethane:methanol, 30:1) to provide the title compound as a grey solid (0.086 g, 76.1% yield).

MS (APCI+) m/z 432 (M+H)⁺; ¹H NMR (DMSO-d₆) δ9.47 (s, 1H), 7.55 (d, 1H, J=7.5 Hz), 7.44 (d, 1H, J=6.9 Hz), 7.23-7.20 (m, 2H), 6.08 (d, 1H, J=7.5 Hz), 5.02 (s, 1H), 4.88 (ABq, 2H, Δν=17.1 Hz, J=18.6 Hz), 2.37 (s, 3H).

### EXAMPLE 44

### 4-(3-bromo-4-fluorophenyl)-3-(2,2-dimethylpropanoyl)-2-(trifluoromethyl)-4,6-dihydro[1,6]naphthyridin-5(1H)-one

The product from Example 24B can be processed as described in Example 4 to provide the title compound.

### Determination of Potassium Channel Opening Activity Membrane Hyperpolarization Assays

Compounds were evaluated for potassium channel opening activity using primary cultured guinea-pig urinary bladder (GPB) cells.

For the preparation of urinary bladder smooth muscle cells, urinary bladders were removed from male guinea-pigs (Hartley, Charles River, Wilmington, Mass.) weighing 300-400 g and placed in ice-cold Ca²⁺-free Krebs solution (Composition, mM: KCl, 2.7; KH₂PO₄, 1.5; NaCl, 75; Na₂HPO₄, 9.6; Na₂HPO₄.7H₂O, 8; MgSO₄, 2; glucose, 5; HEPES, 10; pH 7.4). Cells were isolated by enzymatic dissociation as previously described with minor modifications in Klockner, U. and Isenberg, G., Pflugers Arch. 1985, 405, 329-339, hereby incorporated by reference. The bladder was cut into small sections and incubated in 5 mL of the Kreb's solution containing 1 mg/mL collagenase (Sigma, St. Louis, Mo.) and 0.2 mg/mL pronase (Calbiochem, La Jolla, Calif.) with continuous stirring in a cell incubator for 30 minutes. The mixture was then centrifuged at 1300×g for 5 minutes, and the pellet resuspended in Dulbecco's PBS (GIBCO, Gaithersburg, Md.) and recentrifuged to remove residual enzyme. The cell pellet was resuspended in 5 mL growth media (composition: Dulbecco's modified Eagle's medium supplemented with 10% fetal bovine serum, 100 units/mL penicillin, 100 units/mL streptomycin and 0.25 mg/mL amphotericin B) and further dissociated by pipetting the suspension through a flame-polished Pasteur pipette and passing it through a polypropylene mesh membrane (Spectrum, Houston, Tex.). The cell density was adjusted to 100,000 cells/mL by resuspension in growth media. Cells were plated in clear-bottomed black 96-well plates (Packard) for membrane potential studies at a density of 20,000 cells/well and maintained in a cell incubator with 90% air: 10% CO₂ until confluent. Cells were confirmed to be of smooth muscle type by cytoskeletal staining using a monoclonal mouse anti human-α-smooth muscle actin (Biomeda, Foster City, Calif.).

Functional activity at potassium channels was measured by evaluating changes in membrane potential using the bis-oxonol dye DiBAC(4)₃ (Molecular Probes) in a 96-well cell-based kinetic assay system, Fluorescent Imaging Plate Reader (FLIPR) (K. S. Schroeder et al., J. Biomed. Screen., v. 1 pp. 75-81 (1996)), hereby incorporated by reference. DiBAC(4)₃ is an anionic potentiometric probe which partitions between cells and extracellular solution in a membrane potential-dependent manner. With increasing membrane potential (for example, K⁺ depolarization), the probe fuither partitions into the cell; this is measured as an increase in fluorescence due to dye interaction with intracellular lipids and proteins. Conversely, decreasing membrane potential (hyperpolarization by potassium channel openers) evokes a decrease in fluorescence.

Confluent guinea-pig urinary bladder cells cultured in black clear-bottomed 96-well plates were rinsed twice with 200 mL assay buffer (composition, mM: HEPES, 20; NaCl, 120; KCl, 2; CaCl₂, 2; MgCl₂, 1; glucose, 5; pH 7.4 at 25° C.) containing 5 μM DiBAC(4)₃ and incubated with 180 mL of the buffer in a cell incubator for 30 minutes at 37° C. to ensure dye distribution across the membrane. After recording the baseline fluorescence for 5 minutes, the reference or test compounds, prepared at 10 times the concentration in the assay buffer, were added directly to the wells. Changes in fluorescence were monitored for an additional 25 minutes. Hyperpolarization responses were corrected for any background noise and were normalized to the response observed with 10 μM of the reference compound P1075, N″-cyano-N-(tert-pentyl)-N′-(3-pyridinyl)guanidine, which was assigned as 100%. P1075 is a potent opener of smooth muscle K_{ATP} channels (Quast et al., Mol. Pharmacol., v. 43 pp. 474-481 (1993)) and was prepared using the procedures described in (Manley, J. Med. Chem. (1992) 35, 2327-2340), hereby incorporated by reference.

Routinely, five concentrations of P1075 or test compounds (log or half-log dilutions) were evaluated and the maximal steady-state hyperpolarization values (expressed as % relative to P1075) plotted as a function of concentration. The EC₅₀ (concentration that elicites 50% of the maximal response for the test sample) values were calculated by non-linear regression analysis using a four parameter sigmoidal equation. The maximal response of each compound (expressed as % relative to P1075) is reported. Stock solutions of compounds were prepared in 100% DMSO and further dilutions were carried out in the assay buffer and added to a 96-well plate. The maximal steady-state hyperpolarization values (expressed as % relative to P1075) and the EC₅₀ values for representative compounds of the present invention are shown in Table 1.

TABLE 1
Membrane Hyperpolarization (MHP)
in Guinea-Pig Bladder (GPB) Cells

| | Maximal | |
|---|---|---|
| Example | Response | |
| Number | (% P1075) | EC₅₀(μM) |
| 1 | 83 | 0.052 |
| 2 | 122 | 0.10 |
| 3 | 78 | 2.5 |
| 4 | 92 | 0.013 |
| 5 | 114 | 0.017 |
| 6 | 102 | 1.5 |
| 8 | 97 | 0.038 |
| 9 | 106 | 0.62 |
| 10 | 110 | 0.14 |
| 11 | 105 | 0.023 |
| 12 | 89 | 0.89 |
| 13 | 99 | 0.35 |
| 14 | 84 | 2.6 |
| 15 | 102 | 0.11 |
| 16 | 109 | 0.18 |
| 17 | 45 | 14 |
| 18 | 83 | 0.74 |
| 19 | 104 | 0.62 |
| 20 | 51 | 18 |
| 28 | 82 | 0.022 |
| 29 | 81 | 0.019 |
| 30 | 52 | 4 |
| 31 | 93 | 0.039 |
| 32 | 91 | 0.004 |
| 33 | 65 | 0.028 |
| 34 | 90 | 0.006 |

### In vitro Functional Models

Compounds were evaluated for functional potassium channel opening activity using tissue strips obtained from Landrace pig bladders.

Landrace pig bladders were obtained from female Landrace pigs of 9-30 kg. Landrace pigs were euthanized with an intraperitoneal injection of pentobarbital solution, Somlethal® , J. A. Webster Inc., Sterling, Mass. The entire bladder was removed and immediately placed into Krebs Ringer bicarbonate solution (composition, mM: NaCl, 120; NaHCO₃, 20; dextrose, 11; KCl, 4.7; CaCl₂, 2.5; MgSO₄, 1.5; KH₂PO₄, 1.2; K₂EDTA, 0.01, equilibrated with 5% CO₂/95% O₂ pH 7.4 at 37° C.). Propranolol (0.004 mM) was included in all of the assays to block β-adrenoceptors. The trigonal and dome portions were discarded. Strips 3-5 millimeters (mm) wide and 20 mm long were prepared from the remaining tissue cut in a circular fashion. The mucosal layer was removed. One end was fixed to a stationary glass rod and the other to a Grass FT03 transducer at a basal preload of 1.0 g. Two parallel platinum electrodes were included in the stationary glass rod to provide field stimulation of 0.05 Hz, 0.5 milli-seconds at 20 volts. This low frequency stimulation produced a stable twitch response of 100-500 centigrams. Tissues were allowed to equilibrate for at least 60 minutes and primed with 80 mM KCl. A control concentration response curve (cumulative) was generated for each tissue using the potassium channel opener P1075 as the control agonist. P1075 completely eliminated the stimulated twitch in a dose dependent fashion over a concentration range of 10⁻⁹ to 10⁻⁵ M using ½ log increments. After a 60 minute rinsing period, a concentration response curve (cumulative) was generated for the test agonist in the same fashion as that used for the control agonist P1075. The maximal efficacy of each compounds (expressed as % relative to P1075) is reported. The amount of agent necessary to cause 50% of the agent's maximal response (ED₅₀) was calculated using “ALLFIT” (DeLean et al., Am. J. Physiol., 235, E97 (1980)), and agonist potencies were expressed as p_{D2} (the negative logarithm). Agonist potencies were also expressed as an index relative to P1075. The index was calculated by dividing the ED₅₀ for P1075 by the ED₅₀ for the test agonist in a give tissue. Each tissue was used for only one test agonist, and the indices obtained from each tissue were averaged to provide an average index of potency. These data are shown in Table 2.

TABLE 2
Functional Potassium Channel Opening
Activity in Isolated Bladder Strips

| | Landrace Pig Bladder | | |
|---|---|---|---|
| Example | Efficacy | | |
| Number | (% P1075) | pD₂ | Index |
| 2 | 60 | 6.16 | 0.2653 |
| 5 | 98 | 6.85 | 1.2740 |
| 6 | 67 | 4.89 | 0.0318 |
| 7 | 100 | 7.99 | 2.826 |
| 26 | 98 | 6.56 | 0.6715 |
| 33 | 61 | 5.31 | 0.0353 |
| 34 | 85 | 6.14 | 0.2089 |
| 35 | 77 | 6.10 | 0.3068 |
| 38 | 78 | 6.00 | 0.2488 |

As shown by the data in Tables 1 and 2, the compounds of this invention reduce stimulated contractions of the bladder by opening potassium channels and therefore may have utility in the treatment of diseases prevented by or ameliorated with potassium channel openers.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. In particular, the stereochemistry at the point attachment of R₁, as shown in formula I-V, may independently be either (R) or (S), unless specifically noted otherwise. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Compounds of the present invention may exist as tautomers. The present invention contemplates tautomers due to proton shifts from one atom to another atom of the same molecule generating two distinct compounds that are in equilibrium with each other.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

Further included within the scope of the present invention are pharmaceutical compositions comprising one or more of the compounds of formula I-V prepared and formulated in combination with one or more non-toxic pharmaceutically acceptable compositions. The pharmaceutical compositions can be formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; D absorption accelerators such as quaternary ammonium compounds; g) wetting agents such as cetyl alcohol and glycerol monostearate;) absorbents such as kaolin and bentonite clay; and i) lubricants such as talc, calcium stearate, magnesium stearate, solid polyethylene glycols, sodium lauryl sulfate, and mixtures thereof In the case of capsules, tablets and pills, the dosage form may also comprise buffering agents.

Solid compositions of a similar type may also be employed as fillers in soft and hard- filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The terms “pharmaceutically acceptable salts, esters and amides,” as used herein, refer to carboxylate salts, amino acid addition salts, zwitterions, esters and amides of compounds of formula I-V which are, within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the invention include phosphate, tris and acetate.

The term “pharmaceutically acceptable ester,” as used herein, refers to esters of compounds of the present invention which hydrolyze in vivo and include those that break down readily in the human body to leave the parent compound or a salt thereof Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula I-V may be prepared according to conventional methods.

The term “pharmaceutically acceptable amide,” as used herein, refers to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula I-V may be prepared according to conventional methods.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to the parent compound of the above formula, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987), hereby incorporated by reference.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

The present invention contemplates pharmaceutically active compounds either chemically synthesized or formed by in vivo biotransformation to compounds of formula I-V.

The compounds of the invention, including but not limited to those specified in the examples, possess potassium channel opening activity in mammals (especially humans). As potassium channel openers, the compounds of the present invention may be useful for the treatment and prevention of diseases such as asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina and ischemia.

The ability of the compounds of the present invention, including but not limited to those specified in the examples, to treat bladder overactivity, sensations of incontinence urgency, urinary incontinence, pollakiuria, bladder instability, nocturia, bladder hyerreflexia, and enuresis may be demonstrated by (Resnick, The Lancet (1995) 346, 94-99; Hampel, Urology (1997) 50 (Suppl 6A), 4-14; Bosch, BJU International (1999) 83 (Suppl 2), 7-9; Andersson, Urology (1997) 50 (Suppl 6A), 74-84; Lawson, Pharmacol. Ther., (1996) 70, 39-63; Nurse., Br. J. Urol., (1991) 68, 27-31; Howe, J. Pharmacol. Exp. Ther., (1995) 274, 884-890; Gopalakrishnan, Drug Development Research, (1993) 28, 95-127).

The ability of the compounds of the present invention, including but not limited to those specified in the examples, to treat male sexual dysfunction such as male erectile dysfunction, impotence and premature ejaculation may be demonstrated by (Andersson, Pharmacological Reviews (1993) 45, 253; Lee, Int. J. Impot. Res. (1999) 11(4),179-188; Andersson, Pharmacological Reviews (1993) 45, 253; Lawson, Pharmacol. Ther., (1996) 70, 39-63, Vick, J. Urol. (2000) 163: 202).

The ability of the compounds of the present invention, including but not limited to those specified in the examples, to treat female sexual dysfunction such as clitoral erectile insufficiency, vaginismus and vaginal engorgement may be demonstrated by (J. J. Kim, J. W. Yu, J. G. Lee, D. G. Moon, “Effects of topical K-ATP channel opener solution on clitoral blood flow”, J. Urol. (2000) 163 (4): 240; I. Goldstein and J. R. Berman., “Vasculogenic female sexual dysfunction: vaginal engorgement and clitoral erectile insufficiency syndromes”., Int. J. Impotence Res. (1998) 10:S84-S90).

The ability of the compounds of the present invention, including but not limited to those specified in the examples, to treat benign prostatic hyperplasia (BPH) may be demonstrated by (Pandita, The J. of Urology (1999) 162, 943; Andersson; Prostate (1997) 30: 202-215).

The ability of the compounds of the present invention, including but not limited to those specified in the examples, to treat premature labor and dysmenorrhoea may be demonstrated by (Sanborn, Semin. Perinatol. (1995) 19, 31-40; Morrison, Am. J. Obstet. Gynecol. (1993) 169(5), 1277-85; Kostrzewska, Acta Obstet. Gynecol. Scand. (1996) 75(10), 886-91; Lawson, Pharmacol. Ther., (1996) 70, 39-63).

The ability of the compounds of the present invention, including but not limited to those specified in the examples, to treat functional bowel disorders such as irritable bowel syndrome may be demonstrated by (Lawson, Pharmacol. Ther., (1996) 70, 39-63).

The ability of the compounds of the present invention, including but not limited to those specified in the examples, to treat asthma and airways hyperreactivity may be demonstrated by (Lawson, Pharmacol. Ther., (1996) 70, 39-63; Buchheit, Pulmonary Pharmacology & Therapeutics (1999) 12, 103; Gopalakrishnan, Drug Development Research, (1993) 28, 95-127).

The ability of the compounds of the present invention, including but not limited to those specified in the examples, to treat various pain states including but not limited to migraine and dyspareunia may be demonstrated by (Rodrigues, Br. J. Pharmacol. (2000) 129(1), 110-4; Vergoni, Life Sci. (1992) 50(16), PL135-8; Asano, Anesth. Analg. (2000) 90(5), 1146-51; Lawson, Pharmacol. Ther., (1996) 70, 39-63; Gopalakrishnan, Drug Development Research, (1993) 28, 95-127; Gehlert, Prog. Neuro-Psychopharmacol. & Biol. Psychiat., (1994) 18, 1093-1102).

The ability of the compounds of the present invention, including but not limited to those specified in the examples, to treat epilepsy may be demonstrated by (Lawson, Pharmacol. Ther., (1996) 70, 39-63; Gopalakrishnan, Drug Development Research, (1993) 28, 95-127; Gehlert, Prog. Neuro-Psychopharmacol & Biol. Psychiat., (1994) 18, 1093-1102).

The ability of the compounds of the present invention, including but not limited to those specified in the examples, to treat neurodegenerative conditions and diseases such as cerebral ischemia, stroke, Alzheimer's disease and Parkinson's disease may be demonstrated by (Lawson, Pharmacol. Ther., (1996) 70, 39-63; Gopalakrishnan, Drug Development Research, (1993) 28, 95-127; Gehlert, Prog. Neuro-Psychopharmacol. & Biol. Psychiat., (1994) 18, 1093-1102; Freedman, The Neuroscientist (1996)2, 145).

The ability of the compounds of the present invention, including but not limited to those specified in the examples, to treat diseases or conditions associated with decreased skeletal muscle blood flow such as Raynaud's syndrome and intermittent claudication may be demonstrated by (Lawson, Pharmacol. Ther., (1996) 70, 39-63; Gopalakrishnan, Drug Development Research, (1993) 28, 95-127; Dompeling Vasa. Supplementum (1992) 3434; WO9932495).

The ability of the compounds of the present invention, including but not limited to those specified in the examples, to treat eating disorders such as obesity may be demonstrated by (Spanswick, Nature, (1997) 390, 521-25; Freedman, The Neuroscientist (1996) 2, 145).

The ability of the compounds of the present invention, including but not limited to those specified in the examples, to treat alopecia may be demonstrated by (Lawson, Pharmacol. Ther., (1996) 70, 39-63; Gopalakrishnan, Drug Development Research, (1993) 28, 95-127).

The ability of the compounds of the present invention, including but not limited to those specified in the examples, to treat myocardial injury during ischemia and reperfusion may be demonstrated by (Garlid, Circ Res (1997) 81(6), 1072-82; Lawson, Pharmacol. Ther., (1996) 70, 39-63; Grover, J. Mol. Cell Cardiol. (2000) 32, 677).

The ability of the compounds of the present invention, including but not limited to those specified in the examples, to treat coronary artery disease may be demonstrated by (Lawson, Pharmacol. Ther., (1996) 70, 39-63, Gopalakrishnan, Drug Development Research, (1993) 28, 95-127).

Aqueous liquid compositions of the present invention are particularly useful for the treatment and prevention of asthma, epilepsy, Raynaud's syndrome, male sexual dysfunction, female sexual dysfunction, migraine, pain, eating disorders, urinary incontinence, functional bowel disorders, neurodegeneration and stroke.

When used in the above or other treatments, a therapeutically effective amount of one of the compounds of the present invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal may range from about 0.001 to about 10 mg/kg/day. For purposes of oral administration, more preferable doses can be in the range of from about 0.003 to about 5 mg/kg/day. If desired, the effective daily dose can be divided into multiple doses for purposes of administration; consequently, single dose compositions may contain such amounts or submultiples thereof to make up the daily dose.

What is claimed is:

1. A compound of formula I 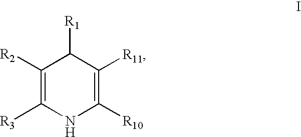
or a pharmaceutically acceptable salt thereof wherein,
R₁ is selected from the group consisting of aryl and heterocycle;
R₂ and R₃, together with the carbon atoms to which each is attached, are a ring selected from the group consisting of 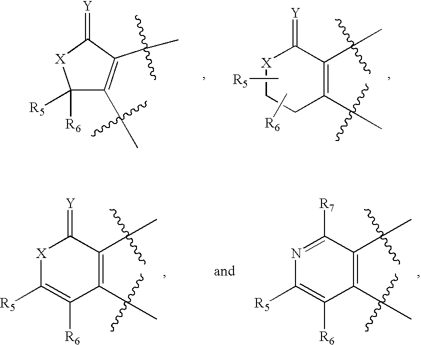
X is selected from the group consisting of O and NR₄;
Y is selected from the group consisting of O and S;
R₄ is selected from the group consisting of hydrogen, alkenyl, alkoxyalkyl, alkoxycarbonylalkyl, alkyl alkylthioalkyl, alkynyl, carboxyalkyl, cyanoalkyl, hydroxyalkyl, mercaptoalkyl, and (NR₈R₉)alkyl wherein R₉ and R₉ are independently selected from the group consisting of hydrogen, alkyl, alkylcarbonyl, and formyl;
R₅ and R₆ are independently selected from the group consisting of hydrogen, alkenyl, alkoxy, alkyl, alkynyl, cyanoalkyl, haloalkyl, and halogen;
R₇ is selected from the group consisting of hydrogen, alkenyloxy, alkenylthio, alkoxy, alkylcarbonylalkoxy, alkylcarbonylalkylthio, alkylcarbonyloxy, alkylcarbonylthio, alkylthio, alkynyloxy, alkynylthio, cyanoalkoxy, cyanoalkylthio, halogen, and —NR₈R₉;
R₁₀ is selected from the group consisting of alkyl, aryl, arylalkyl, haloalkyl, heterocycle, heterocyclealkyl, hydroxyalkyl, and (NR₈R₉)alkyl; and
R₁₁ is selected from the group consisting of hydrogen, alkoxycarbonyl, alkyl, alkylcarbonyl, arylcarbonyl, carboxy, cyano, cyanoalkyl, haloalkyl, and haloalkylcarbonyl;
provided that when R₂ and R₃, together with the carbon atoms to which each is attached, are a ring selected from 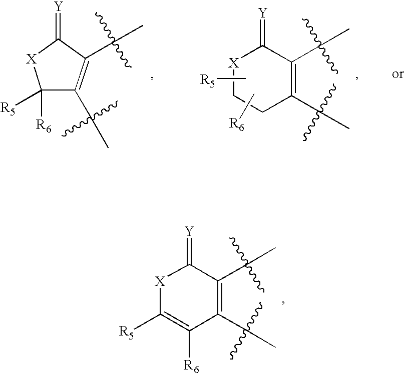
then R₁₁ is other than alkoxycarbonyl or carboxy; and
further provided that when R₂ and R₃, together with the carbon atoms to which each is attached, is 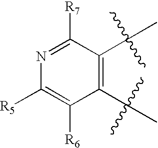
wherein R₅ and R₆ are hydrogen and R₇ is alkoxy, then R₁₁ is other than alkoxycarbonyl or carboxy.

2. A compound according to claim 1 wherein
R₅ and R₆ are independently selected from the group consisting of hydrogen and alkyl;
R₇ is selected from the group consisting of alkoxy, alkylcarbonylalkylthio, alkylthio, and halogen;
R₁₀ is selected from the group consisting of alkyl, aryl, and haloalkyl; and
R₁₁ is selected from the group consisting of alkylcarbonyl, arylcarbonyl, and cyano.

3. A compound according to claim 1 of formula II 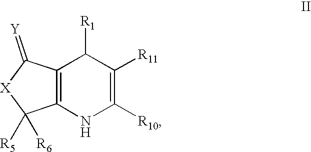
or a pharmaceutically acceptable salt thereof.

4. A compound according to claim 3 wherein
X is NR₄;
Y is O;
R₅ is hydrogen;
R₆ is hydrogen;
R₁₀ is alkyl; and
R₁₁ is cyano.

5. A compound according to claim 4 that is 4-[4-fluoro-3-(trifluoromethyl)phenyl]-3-cyano-2-methyl-1,4,6,7-tetrahydro-5H-pyrrolo[3,4-b]pyridin-5-one.

6. A compound according to claim 1 of formula III 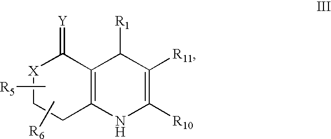
or a pharmaceutically acceptable salt thereof.

7. A compound according to claim 6 wherein
X is NR₄;
Y is O;
R₅ is hydrogen;
R₆ is hydrogen;
R₁₀ is alkyl; and
R₁₁ is cyano.

8. A compound according to claim 7 selected from the group consisting of
4-(3-bromo-4-fluorophenyl)-3-cyano-2-methyl-4,6,7,8-tetrahydro[1,6]naphthyridin-5(1H)-one;
(+) 4-(3-bromo-4-fluorophenyl)-3-cyano-2-methyl-4,6,7,8-tetrahydro[1,6]naphthyridin-5(1H)-one;
(−) 4-(3-bromo-4-fluorophenyl)-3-cyano-2-methyl-4,6,7,8-tetrahydro[1,6]naphthyridin-5(1H)-one;
4-(3,4-dichlorophenyl)-3-cyano-2-methyl-4,6,7,8-tetrahydro[1,6]naphthyridin-5(1H)-one;
4-(3-nitrophenyl)-3-cyano-2-methyl-4,6,7,8-tetrahydro[1,6]naphthyridin-5(1H)-one;
4-(4-chloro-3-nitrophenyl)-3-cyano-2-methyl-4,6,7,8-tetrahydro[1,6]naphthyridin-5(1H)-one;
4-(3,4-dibromophenyl)-3-cyano-2-methyl-4,6,7,8-tetrahydro[1,6]naphthyridin-5(1H)-one;
4-(3,4-difluorophenyl)-3-cyano-2-methyl-4,6,7,8-tetrahydro[1,6]naphthyridin-5(1H)-one;
4-[4-fluoro-3-(trifluoromethyl)phenyl]-3-cyano-2-methyl-4,6,7,8-tetrahydro[1,6]naphthyridin-5(1H)-one;
4-(2,4,5-trifluorophenyl)-3-cyano-2-methyl-4,6,7,8-tetrahydro[1,6]naphthyridin-5(1H)-one;
4-(3-chloro-4-fluorophenyl)-3-cyano-2-methyl-4,6,7,8-tetrahydro[1,6]naphthyridin-5(1H)-one; and
4-[4-chloro-3-(trifluoromethyl)phenyl]-3-cyano-2-methyl-4,6,7,8-tetrahydro[1,6]naphthyridin-5(1H)-one.

9. A compound according to claim 6 wherein
X is NR₄;
Y is O;
R₅ is hydrogen;
R₆ is hydrogen;
R₁₀ is alkyl; and
R₁₁ is alkylcarbonyl.

10. A compound according to claim 9 selected from the group consisting of
3-acetyl-4-(3-bromo-4-fluorophenyl)-2-methyl-4,6,7,8-tetrahydro[1,6]naphthyridin-5(1H)-one; and
4-(3-bromo-4-fluorophenyl)-2-methyl-3-(3-methylbutanoyl)-4,6,7,8-tetrahydro[1,6]naphthyridin-5(1H)-one.

11. A compound according to claim 6 wherein
X is NR₄;
Y is O;
R₅ is hydrogen;
R₆ is hydrogen;
R₁₀ is aryl; and
R₁₁ is arylcarbonyl.

12. A compound according to claim 11 that is 3-benzoyl-4-(3-bromo-4-fluorophenyl)-2-phenyl-4,6,7,8-tetrahydro[1,6]naphthyridin-5(1H)-one.

13. A compound according to claim 6 wherein
X is NR₄;
Y is O;
R₅ is hydrogen;
R₆ is hydrogen;
R₁₀ is haloalkyl; and
R₁₁ is alkylcarbonyl.

14. A compound according to claim 13 that is 4-(3-bromo-4-fluorophenyl)-3-(2,2-dimethylpropanoyl)-2-(trifluoromethyl)-4,6,7,8-tetrahydro[1,6]naphthyridin-5(1H)-one.

15. A compound according to claim 6 wherein
X is O;
Y is O;
R₅ is hydrogen;
R₆ is hydrogen;
R₁₀ is alkyl; and
R₁₁ is cyano.

16. A compound according to claim 15 selected from the group consisting of
4-[4-fluoro-3-(trifluoromethyl)phenyl]-3-cyano-2-methyl-1,4,7,8-tetrahydro-5H-pyrano[4,3-b]pyridin-5-one;
(+) 4-[4-fluoro-3-(trifluoromethyl)phenyl]-3-cyano-2-methyl-1,4,7,8-tetrahydro-5H-pyrano[4,3-b]pyridin-5-one;
(−) 4-[4-fluoro-3-(trifluoromethyl)phenyl]-3-cyano-2-methyl-1,4,7,8-tetrahydro-5H-pyrano[4,3-b]pyridin-5-one; and
4-(3-bromo-4-fluorophenyl)-3-cyano-2-methyl-1,4,7,8-tetrahydro-5H-pyrano[4,3-b]pyridin-5-one.

17. A compound according to claim 1 of formula IV 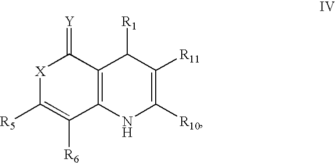
or a pharmaceutically acceptable salt thereof.

18. A compound according to claim 17 wherein
X is NR₄;
Y is O;
R₅ is hydrogen;
R₆ is hydrogen;
R₁₀ is alkyl; and
R₁₁ is cyano.

19. A compound according to claim 18 selected from the group consisting of
4-(3-bromo-4-fluorophenyl)-3-cyano-2-methyl-4,6-dihydro[1,6]naphthyridin-5(1H)-one;
(+) 4-(3-bromo-4-fluorophenyl)-3-cyano-2-methyl-4,6-dihydro[1,6]naphthyridin-5(1H)-one;
(−) 4-(3-bromo-4-fluorophenyl)-3-cyano-2-methyl-4,6-dihydro[1,6]naphthyridin-5(1H)-one;
4-(3-bromo-4-fluorophenyl)-3-cyano-2,6-dimethyl-4,6-dihydro[1,6]naphthyridin-5(1H)-one; and
4-(3-bromo-4-fluorophenyl)-3-cyano-6-(cyanomethyl)-2-methyl-4,6-dihydro[1,6]naphthyridin-5(1H)-one.

20. A compound according to claim 17 wherein
X is NR₄;
Y is O;
R₅ is hydrogen;
R₆ is hydrogen;
R₁₀ is alkyl; and
R₁₁ is alkylcarbonyl.

21. A compound according to claim 20 selected from the group consisting of
3-acetyl-4-(3-bromo-4-fluorophenyl)-2-methyl-4,6-dihydro[1,6]naphthyridin-5(1H)-one;
(+) 3-acetyl-4-(3-bromo-4-fluorophenyl)-2-methyl-4,6-dihydro[1,6]naphthyridin-5(1H)-one;
(−) 3-acetyl-4-(3-bromo-4-fluorophenyl)-2-methyl-4,6-dihydro[1,6]naphthyridin-5(1H)-one; and
4-(3-bromo-4-fluorophenyl)-2-methyl-3-(3-methylbutanoyl)-4,6-dihydro[1,6]naphthyridin-5(1H)-one.

22. A compound according to claim 17 wherein
X is NR;
Y is O;
R₅ is hydrogen;
R₆ is hydrogen;
R₁₀ is haloalkyl; and
R₁₁ is alkylcarbonyl.

23. A compound according to claim 22 that is 4-(3-bromo-4-fluorophenyl)-3-(2,2-dimethylpropanoyl)-2-(trifluoromethyl)-4,6-dihydro[1,6]naphthyridin-5(1H)-one.

24. A compound according to claim 17 wherein
X is NR₄;
Y is O;
R₅ is hydrogen;
R₆ is hydrogen;
R₁₀ is aryl; and
R₁₁ is arylcarbonyl.

25. A compound according to claim 24 that is 3-benzoyl-4-(3-bromo-4-fluorophenyl)-2-phenyl-4,6-dihydro[1,6]naphthyridin-5(1H)-one.

26. A compound according to claim 17 wherein
X is NR₄;
Y is S;
R₅ is hydrogen;
R₆ is hydrogen;
R₁₀ is alkyl; and
R₁₁ is cyano.

27. A compound according to claim 26 that is 4-(3-bromo-4-fluorophenyl)-3-cyano-2-methyl-4,6-dihydro[1,6]naphthyridine-5(1H)-thione.

28. A compound according to claim 1 of formula V 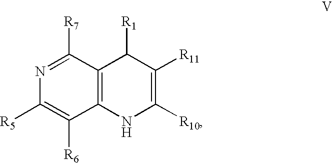
or a pharmaceutically acceptable salt thereof.

29. A compound according to claim 28 wherein
R₅ is hydrogen;
R₆ is hydrogen;
R₁₀ is alkyl; and
R₁₁ is cyano.

30. A compound according to claim 29 selected from the group consisting of
4-(3-bromo-4-fluorophenyl)-5-chloro-3-cyano-2-methyl-1,4-dihydro[1,6]naphthyridine;
(+) 4-(3-bromo-4-fluorophenyl)-5-chloro-3-cyano-2-methyl-1,4-dihydro[1,6]naphthyridine;
(−) 4-(3-bromo-4-fluorophenyl)-5-chloro-3-cyano-2-methyl-1,4-dihydro[1,6]naphthyridine;
4-(3-bromo-4-fluorophenyl)-3-cyano-2-methyl-5-[(2-oxobutyl)sulfanyl]-1,4-dihydro[1,6]naphthyridine;
4-(3-bromo-4-fluorophenyl)-3-cyano-2-methyl-5-(methylsulfanyl)-1,4-dihydro[1,6]naphthyridine; and
4-(3-bromo-4-fluorophenyl)-3-cyano-2-methyl-5-methoxy-1,4-dihydro[1,6]naphthyridine.

31. A compound according to claim 28 wherein
R₅ is hydrogen;
R₆ is hydrogen;
R₇ is halogen;
R₁₀ is alkyl; and
R₁₁ is alkoxycarbonyl.

32. A compound according to claim 31 selected from the group consisting of
4-(3-bromo-4-fluorophenyl)-5-chloro-3-(methoxycarbonyl)-2-methyl-1,4-dihydro[1,6]naphthyridine;
4-(3-bromo-4-fluorophenyl)-5-chloro-3-(ethoxycarbonyl)-2-methyl-1,4-dihydro[1,6]naphthyridine;
(+) 4-(3-bromo-4-fluorophenyl)-5-chloro-3-(ethoxycarbonyl)-2-methyl-1,4-dihydro[1,6]naphthyridine; and
(−) 4-(3-bromo-4-fluorophenyl)-5-chloro-3-(ethoxycarbonyl)-2-methyl-1,4-dihydro[1,6]naphthyridine.

33. A pharmaceutical composition comprising a therapeutically effective amount of a compound of claim 1 in combination with a pharmaceutically acceptable carrier.

34. A method of treating a disorder in a host mammal in need of such treatment comprising administering to the mammal a therapeutically effective amount of a compound of claim 1.

35. The method of claim 34 wherein the disorder is selected from the group consisting of asthma, epilepsy, Raynaud's syndrome, intermittent claudication, migraine, pain, pollakiuria, bladder instability, nocturia, bladder hyperreflexia, enuresis, alopecia, cardioprotection, ischemia, eating disorders, functional bowel disorders, and neurodegeneration.

36. The method of claim 34 wherein the disorder is bladder overactivity.

37. The method of claim 34 wherein the disorder is benign prostatic hyperplasia.

38. The method of claim 34 wherein the disorder is dysmenorrhea.

39. The method of claim 34 wherein the disorder is premature labor.

40. The method of claim 34 wherein the disorder is urinary incontinence.

41. The method of claim 34 wherein the disorder is selected from the group consisting of male erectile dysfunction and premature ejaculation.

42. The method of claim 34 wherein the disorder is female sexual dysfunction.
